(19) United States
(12) Patent Application Publication
(10) Pub. No.: US 2026/0248774 A1
(43) Pub. Date: Aug. 27, 2026

(54) INHIBITORS OF BACTERIAL TYPE III SECRETION SYSTEM

(71) Applicants: MICROBIOTIX, INC., Worcester, MA (US); INFEX THERAPEUTICS LIMITED, Macclesfield (GB)

(72) Inventors: Donald T. Moir, Concord, MA (US); Samanthi L. Waidyarachchi, Worcester, MA (US); Matthew C. Torhan, Worcester, MA (US); Narendran G. Dayanandan, East Longmeadow, MA (US); Sharmila Adhikari, Billerica, MA (US); Zachary D. Aron, Acton, MA (US); Scott Nolan, Northborough, MA (US); Steven Kwasny, Blackstone, MA (US); Steven Cardinale, Hopedale, MA (US); Timothy J. Opperman, Arlington, MA (US); Ian Cooper, Liverpool (GB); James Kirkham, Stockport (GB); Adam Bunt, Macclesfield (GB); Kevin Blades, Ramsbottom (GB)

(73) Assignee: MICROBIOTIX, INC., Worcester, MA (US)

(21) Appl. No.: 18/872,669

(22) PCT Filed: Jun. 5, 2023
(86) PCT No.: PCT/US2023/024416

§ 371 Date: Dec. 6, 2024

Related U.S. Application Data
(60) Provisional application No. 63/349,173, filed on Jun. 6, 2022.

(51) Int. Cl. A61K 31/4439; A61K 31/44; A61K 31/4427; A61K 31/496; A61K 31/5377; A61P 31/04

(52) U.S. Cl. CPC A61K 31/4439; A61K 31/44; A61K 31/4427; A61K 31/496; A61K 31/5377; A61P 31/04

(57) ABSTRACT

Organic compounds showing the ability to inhibit effector toxin secretion or translocation mediated by bacterial type III secretion systems are disclosed. The disclosed type III secretion system inhibitor compounds are useful for combating infections by Gram-negative bacteria such as Pseudomonas aeruginosa.

### STATEMENT REGARDING FEDERALLY SPONSORED RESEARCH

The invention described herein was supported by DHHS/NIH grants No. R43 AI068185 and R01 AI099269 from the National Institutes of Allergy and Infectious Diseases (NIAID). Accordingly, the United States Government has certain rights in the invention.

### FIELD OF THE INVENTION

This invention is in the field of therapeutic drugs to treat bacterial infection and disease. In particular, the invention provides organic compounds that inhibit the type III secretion system of bacterial species.

### BACKGROUND OF THE INVENTION

The bacterial type III secretion system (T3SS) is a complex multi-protein apparatus that facilitates the secretion and translocation of effector proteins from the bacterial cytoplasm directly into the mammalian cytosol. This complex protein delivery device is shared by over 15 species of Gram-negative human pathogens, including Salmonella spp., Shigella flexneri, Pseudomonas aeruginosa, Yersinia spp., enteropathogenic and enteroinvasive Escherichia coli, and Chlamydia spp. (Hueck, 1998, Type III protein secretion systems in bacterial pathogens of animals and plants, Microbiol. Mol. Biol. Rev., 62:379-433; Keyser, et al., 2008, Virulence blockers as alternatives to antibiotics: type III secretion inhibitors against Gram-negative bacteria, J. Intern. Med., 264:17-29.) In the opportunistic pathogen P. aeruginosa, the T3SS is the major virulence factor contributing to the establishment and dissemination of acute infections (Hauser, 2009, The type III secretion system of Pseudomonas aeruginosa: infection by injection, Nat. Rev. Microbiol., 7:654-65). Four T3SS effectors have been identified in P. aeruginosa strains—ExoS, ExoT, ExoY, and ExoU. ExoS and ExoT are bifunctional proteins consisting of an N-terminal small G-protein activating protein (GAP) domain and a C-terminal ADP ribosylation domain; ExoY is an adenylate cyclase; and ExoU is a phospholipase (see review in Engel and Balachandran, 2009, Role of Pseudomonas aeruginosa type III effectors in disease, Curr. Opin. Microbiol., 12:61-6).

In studies with strains producing each effector separately, ExoU and ExoS contributed significantly to persistence, dissemination, and mortality while ExoT produced minor effects on virulence in a mouse lung infection model, and ExoY did not appear to play a major role in the pathogenesis of P. aeruginosa (Shaver and Hauser, 2004, Relative contributions of Pseudomonas aeruginosa ExoU, ExoS, and ExoT to virulence in the lung, Infect. Immun., 72:6969-77). While not a prototypical effector toxin, flagellin (FliC) may also be injected into the cytoplasm of host cells from P. aeruginosa via the T3SS machinery, where it triggers activation of the innate immune system through the nod-like receptor NLRC4 inflammasome. (Franchi, et al., 2009, The inflammasome: a caspase-1-activation platform that regulates immune responses and disease pathogenesis, Nat. Immunol., 10:241-7; Miao, et al., 2008, Pseudomonas aeruginosa activates caspase 1 through Ipaf, Proc. Natl. Acad. Sci. USA, 105:2562-7.)

The presence of a functional T3SS is significantly associated with poor clinical outcomes and death in patients with lower respiratory and systemic infections caused by P. aeruginosa (Roy-Burman, et al., 2001, Type III protein secretion is associated with death in lower respiratory and systemic Pseudomonas aeruginosa infections, J. Infect. Dis., 183:1767-74). In addition, T3SS reduces survival in P. aeruginosa animal infection models (Schulert, et al., 2003, Secretion of the toxin ExoU is a marker for highly virulent Pseudomonas aeruginosa isolates obtained from patients with hospital-acquired pneumonia, J. Infect. Dis., 188:1695-706), and is required for the systemic dissemination of P. aeruginosa in a murine acute pneumonia infection model (Vance, et al., 2005, Role of the type III secreted exoenzymes S, T, and Y in systemic spread of Pseudomonas aeruginosa PAO1 in vivo, Infect. Immun., 73:1706-13). T3SS appears to contribute to the development of severe pneumonia by inhibiting the ability of the host to contain and clear bacterial infection of the lung. Secretion of T3SS toxins, particularly ExoU, blocks phagocyte-mediated clearance at the site of infection and facilitates establishment of an infection (Diaz, et al., 2008, Pseudomonas aeruginosa induces localized immuno-suppression during pneumonia, Infect. Immun., 76:4414-21). The result is a local disruption of an essential component of the innate immune response, which creates an environment of immunosuppression in the lung. This not only allows P. aeruginosa to persist in the lung, but it also facilitates superinfection with other species of bacteria.

While several antibacterial agents are effective against P. aeruginosa, the high rates of mortality, ranging from 40% up to 69% (Garnacho-Montero, et al., 2007, Optimal management therapy for Pseudomonas aeruginosa ventilator-associated pneumonia: an observational, multicenter study comparing monotherapy with combination antibiotic therapy. Crit. Care Med., 35, 1888-1895), and relapse, greater than 30% (Neuhauser, et al., 2003, Antibiotic resistance among gram-negative bacilli in US intensive care units: implications for fluoroquinolone use. JAMA, 289, 885-888), associated with serious P. aeruginosa infections, even in patients with hospital-acquired pneumonia (HAP) and ventilator-associated pneumonia (VAP) receiving antibiotics active against the causative strain, reflect the increasing incidence of drug-resistant strains and highlights the need for new therapeutic agents. (See, e.g., El Solh, et al., 2007, Clinical and hemostatic responses to treatment in ventilator-associated pneumonia: role of bacterial pathogens, Crit. Care Med., 35:490-6; Garnacho-Montero et al., 2007, op cit; Rello, et al., 1998, Recurrent Pseudomonas aeruginosa pneumonia in ventilated patients: relapse or reinfection?, Am. J. Respir. Crit. Care Med., 157:912-6; and Silver, et al., 1992, Recurrent Pseudomonas aeruginosa pneumonia in an intensive care unit, Chest, 101:194-8.) Conventional bacteriostatic and bactericidal antibiotics are insufficient to adequately combat these infections, and new treatment approaches such as inhibitors of P. aeruginosa virulence determinants may prove useful as adjunctive therapies (Veesenmeyer, et al., 2009, Pseudomonas aeruginosa virulence and therapy: evolving translational strategies, Crit. Care Med., 37:1777-86.)

The potential for the type III secretion system as a therapeutic target has prompted several groups to screen for inhibitors of T3SS in various bacterial species, including Salmonella typhimurium, Yersinia pestis, Y. pseudotuberculosis, and E. coli. (Reviewed in Kang, et al., 2021. High-Throughput Approaches for the Identification of Pseudomonas aeruginosa Antivirulents. mBio 12, DOI 10.1128/mBio.02240-20; Keyser, et al., 2008, Virulence blockers as alternatives to antibiotics: type III secretion inhibitors against Gram-negative bacteria, J. Intern. Med., 264:17-29; and Clatworthy, et al., 2007, Targeting virulence: a new paradigm for antimicrobial therapy, Nat. Chem. Biol., 3:541-8). High levels of sequence conservation among various proteins comprising the T3SS apparatus suggest that inhibitors of T3SS in one species may also be active in related species. Broad spectrum activity of T3SS inhibitors identified in a screen against Yersinia has been demonstrated in Salmonella, Shigella, and Chlamydia. Hudson, et al., 2007, Inhibition of type III secretion in Salmonella enterica serovar Typhimurium by small-molecule inhibitors, Antimicrob. Agents Chemother., 51:2631-5; Veenendaal, et al., 2009, Small-molecule type III secretion system inhibitors block assembly of the Shigella type III secreton, J. Bacteriol., 191:563-70; Wolf, et al., 2006, Treatment of Chlamydia trachomatis with a small molecule inhibitor of the Yersinia type III secretion system disrupts progression of the chlamydial developmental cycle, Mol. Microbiol., 61:1543-55.

Screening for P. aeruginosa T3SS inhibitors has been reported (see e.g., Moir, et al., 2021, Adjunctive therapy for multidrug-resistant bacterial infections: Type III secretion system and efflux inhibitors, Drug Discov Today doi: 10.1016/j.drudis.2021.03.031), leading to several selective inhibitors of P. aeruginosa T3SS-mediated secretion, one of which reproducibly inhibits both T3SS-mediated secretion and translocation (Aiello, et al., 2010, Discovery and Characterization of Inhibitors of Pseudomonas aeruginosa Type III Secretion, Antimicrob. Agents Chemother., 54 (5): 1988-1999).

Nevertheless, there remains a medical need for optimized inhibitors of bacterial T3SS of P. aeruginosa with drug-like properties. Specifically, inhibitors are needed which are easily prepared in clinically acceptable formulations, are safe and efficacious in animal models of infection and exhibit adequate pharmacokinetic parameters ensuring sufficient inhibitor levels in appropriate tissues.

### SUMMARY OF THE INVENTION

The present invention provides novel antibacterial/antivirulence agents active against current drug-resistant strains of P. aeruginosa. The compounds of the invention demonstrate a surprising and unexpected improved level of potency, aqueous solubility, in vivo pharmacokinetics, safety, and efficacy in comparison to known T3SS inhibitor compounds which makes them drug-like promising additions to the developing family of antibacterial/antivirulence agents.

The present invention provides new bacterial type III secretion system (T3SS) inhibitor compounds. The T3SS inhibitory compounds described herein were identified through a program to make structural modifications on a phenoxyacetamide (PhA) scaffold, and then to test the novel analogs using cell-based secretion, translocation and cytotoxicity assays. The present invention provides additional compounds with surprisingly improved T3SS inhibition potencies, solubility, in vivo tolerability, in vivo stability, lung and other tissue levels, and in vivo efficacy in a murine lung infection model, as compared to previous phenoxyacetamides or other small molecule T3SS inhibitors.

Without being limited, the surprisingly improved drug-like properties of the compounds disclosed herein may be related to their zwitterionic nature. As reported in Aiello, et al., 2010, op. cit., structure/activity relationship (SAR) studies based on the compound designated MBX-1641, i.e., N-(benzo[d][1,3]dioxol-5-ylmethyl)-2-(2,4-dichlorophenoxy)propanamide, having the formula

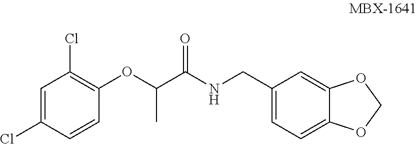 [SMILES: CC(Oc1ccc(Cl)cc1Cl)C(=O)NCc1ccc2c(c1)OCO2]

led to the synthesis of additional T3SS inhibitor analogs, including MBX-2359 (Bowlin, et al., 2014, Mutations in the Pseudomonas aeruginosa Needle Protein Gene pscF Confer Resistance to Phenoxyacetamide Inhibitors of the Type III Secretion System. Antimicrob. Agents Chemother., 58:2211-20), but none that exhibited properties of solubility, potency, stability, selectivity and in vivo pharmacokinetics, safety, and efficacy so promising as those of the current invention for development as drugs.

The present invention is the result of further SAR study of the phenoxyacetamide scaffold. The results provide novel analogs with zwitterion moieties to provide polarity under appropriate conditions for solubility, formulation and delivery to the patient or animal model, but allow the compounds to be less polar under other conditions such as binding to the T3SS needle target. Optimization of the zwitterion component, its structure and position in the analog, have provided new T3SS inhibitors with superior potency and also superior drug-like properties for delivery and efficacy.

The new analogs of this invention provide superior physical and ADMET properties with respect to the prototypical inhibitor scaffolds represented by MBX-1641 and MBX-2359. It has been discovered, for example, that a zwitterionic substituent as represented in the formula below,

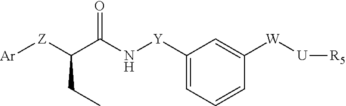

is an optimal substituent on this scaffold and represents a structural distinction from previously studied aryloxyacetamide inhibitor compounds.

Successful drugs must meet a range of criteria establishing their safety, pharmacokinetics (PK) and efficacy. Chemical optimization of the phenoxyacetamide series illustrates the complexity of this process of simultaneously optimizing multiple characteristics. As shown in Table 1, 16 of 18 analogs with various favorable drug-like features fail to meet other critical drug-like features. For example, some are not soluble enough to formulate for delivery of adequate amounts to patients and test animals (e.g., MBX-2263, MBX-2359), some exhibit short half-lives in murine liver microsomal preparations (e.g., MBX-1641, MBX-2625, MBX-2785, MBX-2837, MBX-3337) or short half-lives in mice (e.g., MBX-3458), indicating that they will not be stable enough to demonstrate in vivo efficacy; some exhibit cytotoxicity indicating that they will likely not be safe enough in animal studies or human therapies (e.g., MBX-2624, MBX-2625, MBX-2785, MBX-2867, MBX-5031); some are not specific enough and are potent inhibitors of various mammalian receptors and enzymes, including hERG (e.g., MBX-4532, MBX-4567, MBX-4565); and finally, some are not quite potent enough in their inhibition of T3SS-mediated translocation (e.g., MBX-2264, MBX-2785, MBX-3323, MBX-4567). As is known in the art and is evident from these lists, some analogs fail to meet more than one criterion, but the failure to meet even one is sufficient to block their further development as drugs in most cases. It is also evident that chemical optimization to solve one problem often leads to another problem with analogs. The optimization process is complex and often unpredictable, and yet, the zwitterion series surprisingly meet all of the criteria. The balancing act of improving solubility to enable higher concentrations in formulations for therapy along with minimizing non-specific interactions and toxicities, as well as maximizing tissue levels and maintaining potency is an art. For example, the addition of hydroxyl, amine or carboxylic acid moieties to the phenoxyacetamide scaffold increased polarity and solubility but resulted in unacceptable cytotoxicity (e.g., MBX-2785, MBX-2867, MBX-5031) or overly short half-lives in liver microsomal preparations (e.g., MBX-2785, MBX-2837, MBX-3337) or unacceptable hERG inhibitory activity (MBX-4565). Surprisingly, the addition of a zwitterionic moiety to the phenoxyacetamide scaffold resulted in the identification of several analogs (e.g., MBX-5452A and MBX-6681B) that meet all of the drug-like properties required for developing these compounds into drugs for human or animal therapeutic treatments. Furthermore, the zwitterion analogs MBX-5452A and MBX-6681B exhibit potency, selectivity, and drug-like properties that are superior to those of other patented analogs such as MBX-1641 (US 2014/0219995 A1), which displays lower potency and unacceptably poor MLMt_{1/2} value of <1 min, and MBX-2359 (U.S. Pat. No. 9,340,551 B2), which displays poor aqueous solubility and cannot be formulated to adequately high concentrations in clinically acceptable excipients.

U.S. Pat. No. 9,340,551 B2

TABLE 1

| | | ExoS- | LUX | Trans | | Cytotox- | | hERG |
|---|---|---|---|---|---|---|---|---|
| | | BLA | Sec | location | | HeLa | MLMS | INH |
| MBX- | | IC₅₀ | IC₅₀ | EC₅₀ | Sol | CC₅₀ | T_{1/2} | IC₅₀ |
| ID | Structure | (μM) | (μM) | (μM) | (uM) | (μM) | (min) | (μM) |
| 1641 | 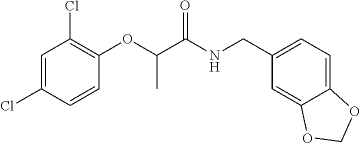 [SMILES: CC(Oc1ccc(Cl)cc1Cl)C(=O)NCc1ccc2c(c1)OCO2] | 10 | 4.7 | 15 | 50 | | <1.00 | |
| 2263 | 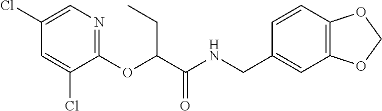 [SMILES: CCC(Oc1ncc(Cl)cc1Cl)C(=O)NCc1ccc2c(c1)OCO2] | 2 | 2.7 | 17 | 25 | >100 | | |
| 2264 | 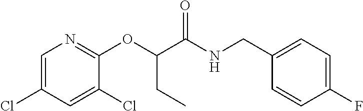 [SMILES: CCC(Oc1ncc(Cl)cc1Cl)C(=O)NCc1ccc(F)cc1] | 2 | 2 | >35 | 200 | 85 | | |
| 2359 | 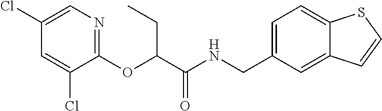 [SMILES: CCC(Oc1ncc(Cl)cc1Cl)C(=O)NCc1ccc2sccc2c1] | 1.5 | 1.3 | 5.2 | 25 | >100 | <1.00 | |
| 2624 | 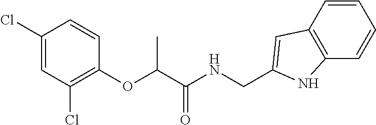 [SMILES: CC(Oc1ccc(Cl)cc1Cl)C(=O)NCc1cc2ccccc2[nH]1] | 9 | 8.7 | 17 | 100 | 9 | | |
| 2625 | 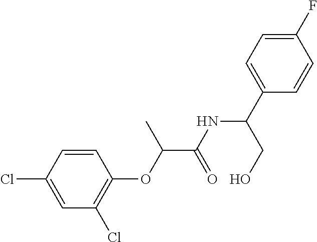 [SMILES: CC(Oc1ccc(Cl)cc1Cl)C(=O)NC(CO)c1ccc(F)cc1] | 5 | 7.1 | 22 | 200 | 40 | 22.1 | |
| 2785 | 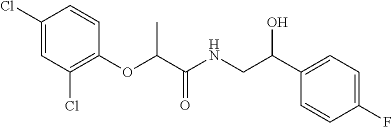 [SMILES: CC(Oc1ccc(Cl)cc1Cl)C(=O)NCC(O)c1ccc(F)cc1] | 15 | 18 | 25 | 400 | 30 | 2.1 | |
| 2837 | 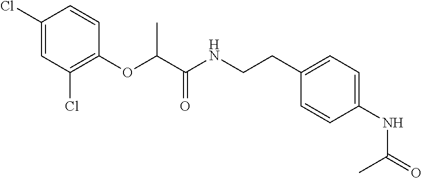 [SMILES: CC(=O)Nc1ccc(CCNC(=O)C(C)Oc2ccc(Cl)cc2Cl)cc1] | 9 | 3.5 | 13 | 200 | >100 | 2.1 | |
| 2867 | 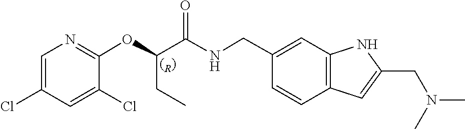 | 1.3 | 1.6 | 5.8 | 3000 | 30 | | |
| 3323 | 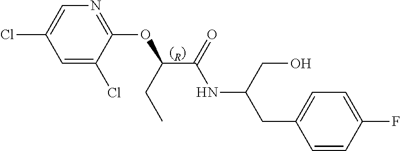 | 5 | 5.2 | 13 | 2500 | >100 | 70 | |
| 3337 | 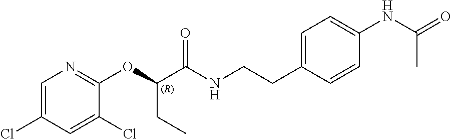 | 1.2 | 1.4 | 4.8 | 60 | >100 | 3 | |
| 3458 | 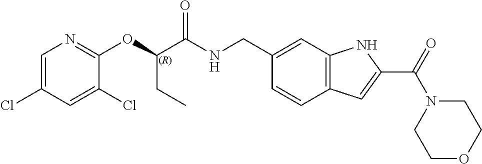 | 0.9 | 3.7 | 5.2 | 25 | 100 | | |
| 4532 | 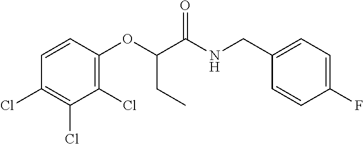 [SMILES: CCC(Oc1ccc(Cl)c(Cl)c1Cl)C(=O)NCc1ccc(F)cc1] | 1 | 1.63 | 1.7 | 25 | >200 | | >100 |
| 4565 | 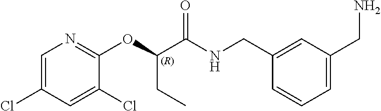 | 9 | 8.3 | 20 | 200 | | 61 | 9.2 |
| 4567 | 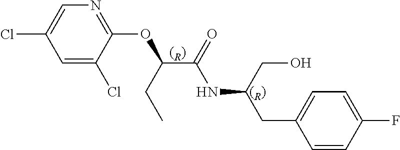 | 14 | 8.5 | 60 | 25 | | | >100 |
| 5031 | 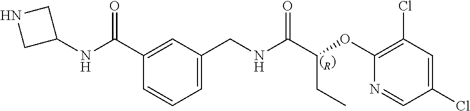 | 1.7 | 0.2 | 1.7 | 50 | 132 | >139 | |
| 5452A | 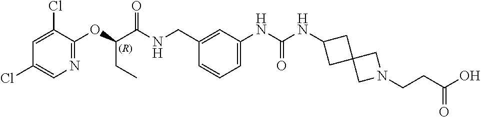 | 0.75 | 0.95 | 2.7 | ≥200 | >200 | >125 | >100 |
| 6681B | 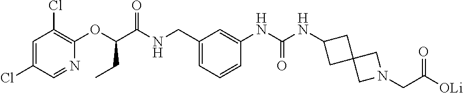 | 0.85 | 1.1 | 2.5 | ≥200 | >200 | >125 | |

Accordingly, the T3SS inhibitor compounds described herein inhibit T3SS-mediated secretion of a bacterial exotoxin (effector) from a bacterial cell. More preferably, a T3SS inhibitor compound described herein inhibits T3SS-mediated secretion of an effector from a bacterial cell and also inhibits T3SS-mediated translocation of the effector from the bacterial cell to a host cell (e.g., human or other animal cell).

In an embodiment, a T3SS inhibitor compound described herein inhibits the T3SS of Pseudomonas and the T3SS of a bacterium of at least one other genus. Preferably, the inhibition target Pseudomonas bacterium is P. aeruginosa.

The present invention provides a novel class of bacterial type III secretion system (T3SS) inhibitor compounds as presented by formula I:

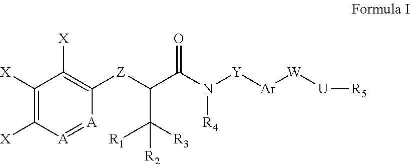

Wherein,
A is independently selected from CH or N;
X is independently selected from hydrogen, halogen or hydroxyl;
Z is O, S, NH; or NR′, wherein R′ is alkyl;
R₁, R₂, and R₃ are independently selected from: hydrogen, halogen, alkyl, hydroxy, alkoxy, alkylthio, or cyano, wherein no more than two of the preceding radicals is hydrogen;
NR₄ wherein;
R₄ is hydrogen, a straight chain aliphatic group, a branched chain aliphatic group, cycloalkyl, haloalkyl, hydroxyalkyl, alkoxy, alkylamino, alkylthio, sulfonyl, sulfinyl, carboxy, alkoxycarbonyl or an aryl;
Y is selected from:
a divalent straight-chain, branched, or cyclic alkyl, alkenyl or alkynyl radical of from 1 to 6 carbon atoms, which may contain one or more heteroatoms, and which may be unsubstituted or substituted with up to four substituents selected from halo, cyano, hydroxy, amino, alkyl, cycloalkyl, alkylamino, carboxyl, alkoxycarbonyl, carboxamido, acylamino, amidino, sulfonamide, aminosulfonyl, alkylsulfonyl, aryl, heteroaryl, alkoxy, alkylthio; aryloxy, and heteroaryloxy; oxygen; oxime; or NR″ where R″ is hydrogen, alkyl or cycloalkyl;
Ar is an aryl or heteroaryl radical forming a five-membered or six-membered ring which may be additionally fused with from 1 to 3 aryl, heteroaryl, cycloalkyl, or heterocycloalkyl rings, which Ar radical may be unsubstituted or substituted with up to four substituents selected from halo, cyano, hydroxy, amino, alkyl, cycloalkyl, alkylamino, carboxyl, alkoxycarbonyl, carboxamido, acylamino, amidino, sulfonamido, aminosulfonyl, alkylsulfonyl, aryl, heteroaryl, alkoxy, alkylthio; aryloxy, and heteroaryloxy, and wherein any two substituents together may form an aromatic or non-aromatic ring structure fused with said aryl or heteroaryl radical Ar, or alternatively wherein substituents on Ar also may be optionally bonded covalently to either Y or R₄, or both Y and R₄, to form heterocyclic or carbocyclic ring systems, which ring systems may be aromatic, heteroaromatic, or partly aromatic, (that is, one or more rings being aromatic and one or more rings being non-aromatic (saturated));
W is a divalent radical bridging Ar and U selected from the group comprising, —COCH₂—, —SO₂—, —NHSO₂—, —SO2NH—, —CO—, —CH₂—, —CH(CH₃)—, —NHCO—, —NHCONH— —NCH₃CO—, —CONH—, —CONCH₃—, —O(CO)—, —(CO)O—, —NH—, or —O—;
U can be either a nitrogen or a carbon that is part of a non-aromatic heterocyclic ring of from 4-7 members and containing 1-2 nitrogen atoms, 0-1 oxygen atoms and bearing 0-3 substituents (in addition to the bond between W and the ring carbon/nitrogen and the U and the ring carbon in the formula) selected from alkyl, cycloalkyl, aryl, heteroaryl, haloalkyl, nitro, halogen, alkoxy, alkylthio, haloalkoxy, sulfonyl, or sulfinyl; and additionally U can be either a nitrogen or a saturated carbon that is linked via a chain of 1-4 carbon atoms to a basic nitrogen bearing, independently, hydrogen or aliphatic groups of less than nine carbon atoms that can be optionally substituted with groups selected from haloalkyl, nitro, halogen, alkoxy, alkylthio, haloalkoxy, sulfonyl, sulfinyl, carboxy, alkoxycarbonyl, or aminocarbonyl groups, heteroaryl, haloalkyl, nitro, halogen, alkoxy, alkylthio, haloalkoxy, sulfonyl, or sulfinyl;
R₅ is a monovalent radical selected from the group comprising, —CH2C(CH3)2COOH, —CH2COOH, —CH2COOH, —CH2CH2CH2COOH, —(CyBut)COOH, —CH2(CyPropyl)COOH, CH2(CyPropyl)COOH, —SO₂Me, —SO₂OH, —SO₂CF₃

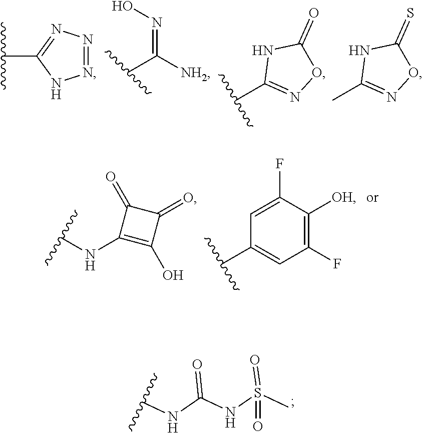

In another embodiment, the present invention provides a family of bacterial type III secretion system (T3SS) inhibitor compounds having the structure of Formula I(a):

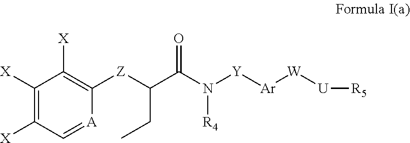

A is independently selected from CH or N;
X is independently selected from hydrogen or halogen;
Z is O, S, NH; or NR′, wherein R′ is alkyl;
R₄ is hydrogen or methyl;
Y is selected from:
a divalent straight-chain, branched, or cyclic alkyl, alkenyl or alkynyl radical of from 1 to 6 carbon atoms, which may contain one or more heteroatoms, and which may be unsubstituted or substituted with up to four substituents selected from halo, cyano, hydroxy, amino, alkyl, cycloalkyl, alkylamino, carboxyl, alkoxycarbonyl, carboxamido, acylamino, amidino, sulfonamide, aminosulfonyl, alkylsulfonyl, aryl, heteroaryl, alkoxy, alkylthio; aryloxy, and heteroaryloxy; oxygen; oxime; or NR″ where R″ is hydrogen, alkyl or cycloalkyl;
Ar is an aryl or heteroaryl divalent radical forming a five-membered or six-membered ring which may be additionally fused with from 1 to 3 aryl, heteroaryl, cycloalkyl, or heterocycloalkyl rings, which Ar radical may be unsubstituted or substituted with up to four substituents selected from halo, cyano, hydroxy, amino, alkyl, cycloalkyl, alkylamino, carboxyl, alkoxycarbonyl, carboxamido, acylamino, amidino, sulfonamido, aminosulfonyl, alkylsulfonyl, aryl, heteroaryl, alkoxy, alkylthio; aryloxy, and heteroaryloxy, and wherein any two substituents together may form an aromatic or non-aromatic ring structure fused with said aryl or heteroaryl radical Ar, or alternatively wherein substituents on Ar also may be optionally bonded covalently to either Y or R₄, or both Y and R₄, to form heterocyclic or carbocyclic ring systems, which ring systems may be aromatic, heteroaromatic, or partly aromatic, (that is, one or more rings being aromatic and one or more rings being non-aromatic (saturated)).
W is a divalent radical bridging Ar and U selected from the group comprising, —COCH₂—, —SO₂—, —NHSO₂—, —SO2NH—, —CO—, —CH₂—, —CH(CH₃)—, —NHCO—, —NHCONH— —NCH₃CO—, —CONH—, —CONCH₃—, —O(CO)—, —(CO)O—, —NH—, or —O—;
U can be either a nitrogen or a carbon that is part of a non-aromatic heterocyclic ring system of between 4-7 members and containing 1-2 nitrogen atoms, 0-1 oxygen atoms and bearing 0-3 substituents (in addition to the bond between W and the ring carbon/nitrogen and the U and the ring carbon in the formula) selected from alkyl, cycloalkyl, aryl, heteroaryl, haloalkyl, nitro, halogen, alkoxy, alkylthio, haloalkoxy, sulfonyl, or sulfinyl; and additionally W can be either a nitrogen or a saturated carbon that is linked via a chain of 1-4 carbon atoms to a basic nitrogen bearing, independently, hydrogen or aliphatic groups of less than nine carbon atoms that can be optionally substituted with groups selected from haloalkyl, nitro, halogen, alkoxy, alkylthio, haloalkoxy, sulfonyl, sulfinyl, carboxy, alkoxycarbonyl, or aminocarbonyl groups, heteroaryl, haloalkyl, nitro, halogen, alkoxy, alkylthio, haloalkoxy, sulfonyl, or sulfinyl;
R₅ is a monovalent radical selected from the group comprising, —CH2C(CH3)2COOH, —CH2COOH, —CH2COOH, —CH2CH2CH2COOH, —(CyBut)COOH, —CH2 (CyPropyl)COOH, CH2(CyPropyl)COOH, —SO₂Me, —SO₂OH, —SO₂CF₃

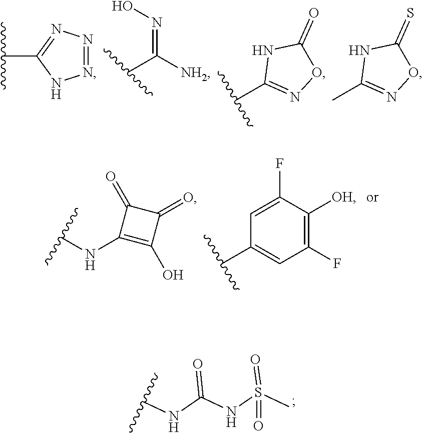

In another embodiment, the present invention provides a family of bacterial type III secretion system (T3SS) inhibitor compounds having the structure of Formula I(b):

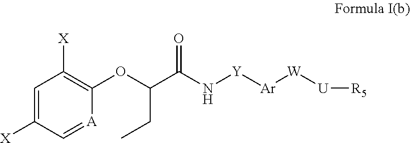

A is CH or N;
X is independently selected from hydrogen or halogen;
Y is —CH₂—, —CH(CH₃)—, or —C(CH₃)₂—; and
Ar is an aryl or heteroaryl divalent radical forming a five-membered or six-membered ring which may be additionally fused with from 1 to 3 aryl, heteroaryl, cycloalkyl, or heterocycloalkyl rings, which Ar radical may be unsubstituted or substituted with up to four substituents selected from halo, cyano, hydroxy, amino, alkyl, cycloalkyl, alkylamino, carboxyl, alkoxycarbonyl, carboxamido, acylamino, amidino, sulfonamido, aminosulfonyl, alkylsulfonyl, aryl, heteroaryl, alkoxy, alkylthio; aryloxy, and heteroaryloxy, and wherein any two substituents together may form an aromatic or non-aromatic ring structure fused with said aryl or heteroaryl radical Ar, or alternatively wherein substituents on Ar also may be optionally bonded covalently to Y; to form heterocyclic or carbocyclic ring systems, which ring systems may be aromatic, heteroaromatic, or partly aromatic, (that is, one or more rings being aromatic and one or more rings being non-aromatic (saturated)).
W is a divalent radical bridging Ar and U selected from the group comprising, —COCH₂—, —SO₂—, —NHSO₂—, —SO2NH—, —CO—, —CH₂—, —CH(CH₃)—, —NHCO—, —NHCONH— —NCH₃CO—, —CONH—, —CONCH₃—, —O(CO)—, —(CO)O—, —NH—, or —O—;
U can be either a nitrogen or a carbon that is part of a non-aromatic heterocyclic ring system of between 4-7 members and containing 1-2 nitrogen atoms, 0-1 oxygen atoms and bearing 0-3 substituents (in addition to the bond between W and the ring carbon/nitrogen and the U and the ring carbon in the formula) selected from alkyl, cycloalkyl, aryl, heteroaryl, haloalkyl, nitro, halogen, alkoxy, alkylthio, haloalkoxy, sulfonyl, or sulfinyl; and additionally W can be either a nitrogen or a saturated carbon that is linked via a chain of 1-4 carbon atoms to a basic nitrogen bearing, independently, hydrogen or aliphatic groups of less than nine carbon atoms that can be optionally substituted with groups selected from haloalkyl, nitro, halogen, alkoxy, alkylthio, haloalkoxy, sulfonyl, sulfinyl, carboxy, alkoxycarbonyl, or aminocarbonyl groups, heteroaryl, haloalkyl, nitro, halogen, alkoxy, alkylthio, haloalkoxy, sulfonyl, or sulfinyl;
R₅ is a monovalent radical selected from the group comprising, —CH2C(CH3)2COOH, —CH2COOH, —CH2COOH, —CH2CH2CH2COOH, —(CyBut)COOH, —CH2(CyPropyl)COOH, CH2(CyPropyl)COOH, —SO₂Me, —SO₂OH, —SO₂CF₃

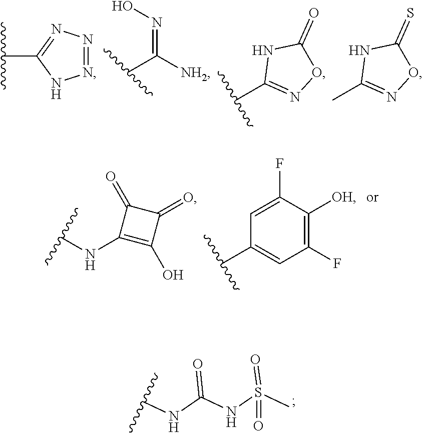

In another embodiment, the present invention provides a family of bacterial type III secretion system (T3SS) inhibitor compounds having the structure of Formula I(c):

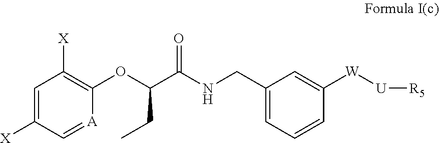

A is CH or N;
at least one X is Cl and the other X is hydrogen, F, or Cl;
W is a divalent radical bridging Ar and U selected from the group comprising, —COCH₂—, —SO₂—, —NHSO₂—, —SO2NH—, —CO—, —CH₂—, —CH(CH₃)—, —NHCO—, —NHCONH— —NCH₃CO—, —CONH—, —CONCH₃—, —O(CO)—, —(CO)O—, —NH—, or —O—;
U can be either a nitrogen or a carbon that is part of a non-aromatic heterocyclic ring system of between 4-7 members and containing 1-2 nitrogen atoms, 0-1 oxygen atoms and bearing 0-3 substituents (in addition to the bond between W and the ring carbon/nitrogen and the U and the ring carbon in the formula) selected from alkyl, cycloalkyl, aryl, heteroaryl, haloalkyl, nitro, halogen, alkoxy, alkylthio, haloalkoxy, sulfonyl, or sulfinyl; and additionally W can be either a nitrogen or a saturated carbon that is linked via a chain of 1-4 carbon atoms to a basic nitrogen bearing, independently, hydrogen or aliphatic groups of less than nine carbon atoms that can be optionally substituted with groups selected from haloalkyl, nitro, halogen, alkoxy, alkylthio, haloalkoxy, sulfonyl, sulfinyl, carboxy, alkoxycarbonyl, or aminocarbonyl groups, heteroaryl, haloalkyl, nitro, halogen, alkoxy, alkylthio, haloalkoxy, sulfonyl, or sulfinyl;
R₅ is a monovalent radical selected from the group comprising, —CH2C(CH3)2COOH, —CH2COOH, —CH2COOH, —CH2CH2CH2COOH, —(CyBut)COOH, —CH2(CyPropyl)COOH, CH2(CyPropyl)COOH, —SO₂Me, —SO₂OH, —SO₂CF₃

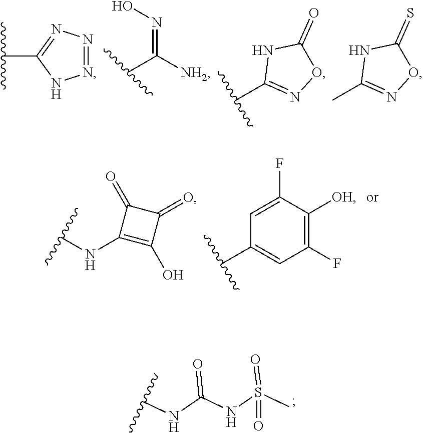

Compounds according to the foregoing formulae were tested using assays showing specific inhibition of the T3SS of P. aeruginosa.

T3SS inhibitor compounds described herein inhibit T3SS effector transcription by at least 15% at a concentration of 50 μM using a transcriptional reporter assay or exhibit at least 50% inhibition of effector secretion at a concentration of 100 μM or less (IC₅₀≤100 μM) in an effector secretion assay. The compounds described above show T3SS-specific inhibition in Pseudomonas of greater than 15% using an exoT-lux transcriptional reporter construct transferred into Pseudomonas aeruginosa PAO1 (reporter strain MDM852, described herein) and/or show an IC₅₀ of less than 100 μM for T3SS as measured in an assay of T3SS-mediated secretion of an effector toxin-β-lactamase reporter fusion protein assay described herein using P. aeruginosa strain MDM973 (PAK/pUCP24GW-lacI^{Q}-lacPO-exoS::blaM). See Table 1, infra. Compounds inhibiting effector transcription by less than 15% or with an IC₅₀ greater than 10 μM are not generally useful as T3SS inhibitors in the compositions and methods described herein.

In a particularly preferred embodiment, a T3SS inhibitor compound useful in the compositions and methods described herein has an IC₅₀ value of less than 200 μM as measured in a T3SS-mediated effector toxin-β-lactamase reporter fusion protein secretion assay described herein (or comparable assay) and also has a relatively low cytotoxicity toward human cells, such as a CC₅₀ value of greater than or equal to 200 μM (CC₅₀≥200 μM) as measured in a standard cytotoxicity assay as described herein or as employed in the pharmaceutical field for antibiotics. Such standard cytotoxicity assays may employ any human cell typically employed in cytotoxicity assays for antibiotics, including but not limited to, Chinese hamster ovary (CHO) cells, HeLa cells, Hep-2 cells, human embryonic kidney (HEK) 293 cells, 293T cells, and the like.

Even more preferably, a T3SS inhibitor compound described herein has an IC₅₀ value ≤50 μM as measured in a T3SS-mediated effector toxin-β-lactamase reporter fusion protein secretion assay as described herein or in a comparable assay.

In a particularly preferred embodiment of the invention, a T3SS inhibitor compound blocks T3SS-mediated secretion and translocation of one or more toxin effectors from cells of P. aeruginosa, and is safe and achieves sufficiently high levels and of sufficient duration in appropriate animal tissues to exhibit efficacy in animal models of infection such as the murine lung infection model.

Even more preferably, to be useful as a drug, a T3SS inhibitor compound described herein has a translocation inhibition EC₅₀<10 μM vs. 90% of P. aeruginosa clinical strains tested, is stable in human serum (>75% stable for >1 hr), is stable in human liver microsome preparations (t_{1/2}>1 hr), is soluble at concentrations >5 mg/mL in clinically acceptable formulations, and displays efficacy in vivo in an animal model of P. aeruginosa infection.

The T3SS compounds described herein are useful as anti-virulence agents and may be used to treat bacterial infections. Accordingly, an individual infected with or exposed to bacterial infection, especially Pseudomonas, or Chlamydia infection, may be treated by administering to the individual in need an effective amount of a compound according to the invention.

Use of one or more or a combination of the compounds disclosed herein to treat infection by bacteria having a type III secretion system is contemplated herein. Especially, use of one or more or a combination of the above compounds to treat Pseudomonas, or Chlamydia infection is contemplated herein. In particular, use of one or more or a combination of the above compounds for the treatment of Pseudomonas aeruginosa, Yersinia pestis, or Chlamydia trachomatis infections is advantageously carried out by following the teachings herein.

The present invention also provides pharmaceutical compositions containing one or more of the T3SS inhibitor compounds disclosed herein and a pharmaceutically acceptable carrier or excipient. The use of one or more of the T3SS inhibitor compounds in the preparation of a medicament for combating bacterial infection is disclosed.

A T3SS inhibitor compound or combination of T3SS inhibitor compounds described herein may be used as a supporting or adjunctive therapy for the treatment of bacterial infection in an individual (human or other animal). In the case of an individual that is not severely immune compromised, administration of a T3SS inhibitor compound described herein to inhibit the T3SS of bacterial cells in or on an individual may be sufficient to permit the individual's own immune system to effectively clear or kill infecting or contaminating bacteria from the tissue of the individual. Alternatively, a T3SS inhibitor compound described herein may be administered to an individual in conjunction (i.e., in a mixture, sequentially, or simultaneously) with an, antibacterial agent, such as an antibiotic, an antibody, or an immunostimulatory agent, to provide both inhibition of T3SS and inhibition of growth of invading bacterial cells.

In yet another embodiment, a composition comprising a T3SS inhibitor or a combination of T3SS inhibitors described herein may also comprise a second agent (second active ingredient, second active agent) that possesses a desired therapeutic or prophylactic activity other than that of T3SS inhibition. Such a second active agent includes, but is not limited to, an antibiotic, an antibody, an antiviral agent, an anticancer agent, an analgesic agent (e.g., a nonsteroidal anti-inflammatory drug (NSAID), acetaminophen, an opioid, a COX-2 inhibitor), an immunostimulatory agent (e.g., a cytokine), a hormone (natural or synthetic), a central nervous system (CNS) stimulant, an antiemetic agent, an anti-histamine, an erythropoietin, a complement stimulating agent, a sedative, a muscle relaxant agent, an anesthetic agent, an anticonvulsive agent, an antidepressant, an antipsychotic agent, and combinations thereof.

Compositions comprising a T3SS inhibitor described herein may be formulated for administration to an individual (human or other animal) by any of a variety of routes including, but not limited to, intravenous, intramuscular, subcutaneous, intra-arterial, parenteral, intraperitoneal, sublingual (under the tongue), buccal (cheek), oral (for swallowing), topical (epidermis), transdermal (absorption through skin and lower dermal layers to underlying vasculature), nasal (nasal mucosa), intrapulmonary (lungs), intrauterine, vaginal, intracervical, rectal, intraretinal, intraspinal, intrasynovial, intrathoracic, intrarenal, nasojejunal, and intraduodenal.

### BRIEF DESCRIPTION OF THE DRAWINGS

FIG. 1 is a graph illustrating concentration-dependent rescue of CHO cells from T3SS-mediated ExoU cytotoxicity by two phenoxyacetamide compounds, measured as previously described in Aiello, et al., 2010, Antimicrob. Agents Chemother., 54:1988-99. Two compounds according to the invention, MBX-5452A and MBX-6681B, are compared against two previously discovered T3SS inhibitors, designated MBX-1641 and MBX-2359, used as a standard of reference. The results indicate that MBX-5452A and MBX-6681 exhibit a surprising 2-4-fold increase in potency compared to MBX-1641 and MBX-2359.

FIG. 2 is a composite of graphs and tables illustrating the surprisingly favorable pharmacokinetic (PK) of MBX-5452A in mice after administration of single dose of 250 mg/kg via the subcutaneous (SC) or intraperitoneal (IP) routes of administration. FIGS. 2A and C show the concentrations of MBX-5452A in plasma, spleen, ELF, and liver at various times after SC administration, and the calculated PK parameters, respectively. FIGS. 2B and D show the concentrations of MBX-5452A in plasma, spleen, ELF, and liver at various times after IP administration, and the calculated PK parameters, respectively.

FIG. 3 is a composite of graphs and tables illustrating the surprisingly favorable pharmacokinetic (PK) of MBX-6681B in mice after administration of single dose of 50 mg/kg via the intravenous (IV) and 100 mg/kg via subcutaneous (SC) or routes of administration. FIGS. 3A and C show the concentrations of MBX-6681B in plasma, spleen, ELF, and liver at various times after IV administration, and the calculated PK parameters, respectively. FIGS. 2B and D show the concentrations of MBX-6681B in plasma, spleen, ELF, and liver at various times after SC administration, and the calculated PK parameters, respectively.

FIG. 4 illustrates the murine PK of MBX-5452A (Panel A) and MBX-6681B (Panel B) in plasma, ELF, and lung after repeated SC doses of 100 mg/kg TID (q8h) for 40 hours. The results indicate that there is no significant accumulation of compounds in plasma, ELF, or lung after repeated dosing, with peak plasma levels averaging around 100 μg/mL shortly after each administration.

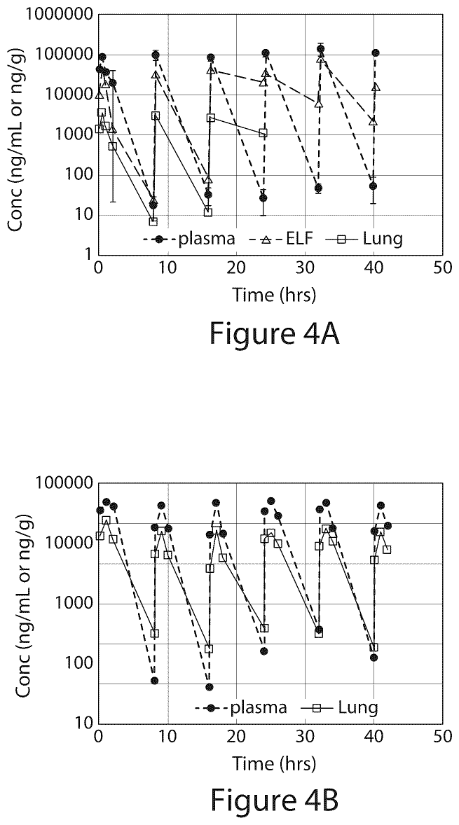

FIG. 5 illustrates the pharmacokinetics of MBX-5452A in plasma of rats after 250 mg/kg doses administered IP or SC (Panel A). The plasma levels of MBX-5452A in mice after SC and IP administration are included for comparison. Panel B is a table containing the PK parameters calculated from the data shown in Panel A, which includes the murine PK parameters for comparison. The data indicate that the PK parameters for rats and mice are similar, except that half-life and MRTINF_obs are significantly higher in rats, and the concentration of MBX-5452A in plasma after SC administration are higher than the T3SS translocation EC₅₀ for >16 hours.

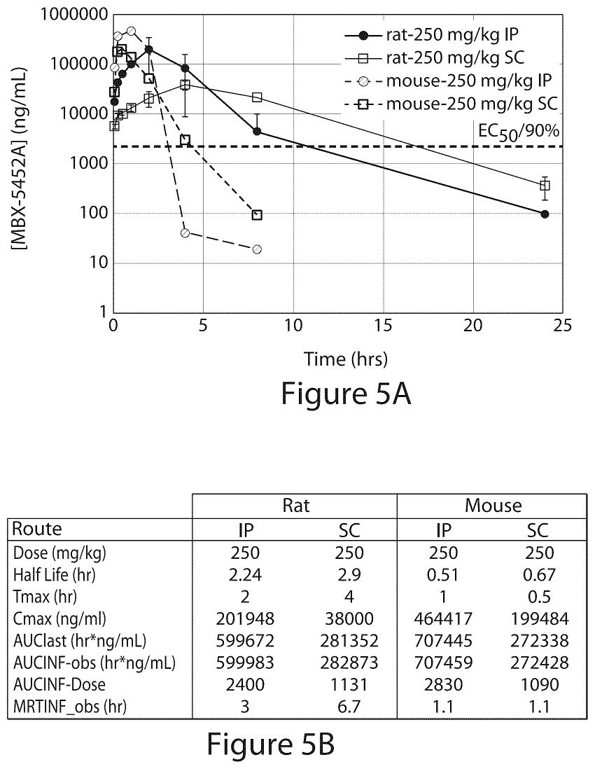

FIG. 6 is comprised of graphs showing that repeat SC or IP doses of 750 mg/kg/day (250 mg/kg, TID of MBX-5452A did not significantly affect body weight (Panel A) or percent change in body weight (Panel B) of CD-1 mice over the course of 4 days. In addition, clinical signs were monitored, and no adverse events were reported, indicating that repeated dosing of MBX-5452A is well-tolerated by mice.

FIG. 7 illustrates that 125 and 250 mg/kg TID doses of MBX-5452A (Panel A) and MBX-6681B (Panel B) significantly decrease bacteria load (colony forming units, CFU) in lungs of immunocompetent CD-1 mice challenged with Pseudomonas aeruginosa PA99 (1×10⁷ CFUs) when administered SC starting at 1-hour post-infection. The log 10 CFU reduction in lung tissue for each treatment group as compared to the vehicle only control group are indicated beneath the data points for each treatment group, and the statistical significance (Mann-Whitney test, P<0.05 indicates significant difference) between selected groups is indicated above the data points. Ciprofloxacin (CIP) dosed a 20 mg/kg SC, TID, was used as the positive control.

FIG. 8 illustrates that 125 and 250 mg/kg TID doses of MBX-5452A (Panel A) and MBX-6681B (Panel B) significantly increase the efficacy of a sub-efficacious dose of meropenem (MEM, 2 mg/kg TID) in decreasing the bacteria load in lungs of immunocompetent CD-1 mice challenged with Pseudomonas aeruginosa PA99 (1×10⁷ CFUs) when administered SC starting at 1-hour post-infection. The log 10 CFU reduction in lung tissue for each treatment group as compared to the vehicle only control group are indicated beneath the data points for each treatment group, and the statistical significance (Mann-Whitney test, P<0.05 indicates significant difference) between selected groups is indicated above the data points. Meropenem (MEM) dosed a 10 mg/kg SC, TID, was used as the positive control. Both compounds exhibited synergistic effects in combination with 2 mg/kg MEM, as the Log 10 reduction in CFU of the combinations were significantly greater than the sum Log 10 reductions of the individual treatments. The combinations zwitterionic T3SS inhibitor and MEM 2 mg/kg reduced CFUs in the lung to levels that are similar to the positive control.

### DETAILED DESCRIPTION OF THE INVENTION

The invention provides organic compounds that inhibit a bacterial type III secretion system (“T3SS”) that secretes and translocates bacterially produced effectors (also referred to as effector toxins, exotoxins, cytotoxins, bacterial toxins) from the bacterial cell into animal host cells. Effectors translocated into host cells can effectively inactivate the host immune response, such as by killing phagocytes (e.g., macrophages and neutrophils) and thereby disabling the host innate immune response. The T3SS is thus a critical virulence factor in establishing bacterial infections in an individual (human or other animal) and is particularly critical to P. aeruginosa opportunistic infections of human patients with compromised immune systems or that otherwise have been made susceptible to infection by bacteria such as P. aeruginosa.

### Abbreviations:

That the invention may be more clearly understood, the following abbreviations and terms are used as defined below.

Abbreviations for various substituents (side groups, radicals) of organic molecules are those commonly used in organic chemistry. Such abbreviations may include “shorthand” forms of such substituents. For example, “Ac” is an abbreviation for an acetyl group, “Ar” is an abbreviation for an “aryl” group, and “halo” or “halogen” indicates a halogen radical (e.g., F, Cl, Br, I). “Me” and “Et” are abbreviations used to indicate methyl (CH₃—) and ethyl (CH₃CH₂—) groups, respectively; and “OMe” (or “MeO”) and “OEt” (or “EtO”) indicate methoxy (CH₃O—) and ethoxy (CH₃CH₂O—), respectively. Hydrogen atoms are not always shown in organic structural diagrams (e.g., at the end of a drawn line representing a CH₃ group) or may be only selectively shown in some structural diagrams, as the presence and location of hydrogen atoms in organic molecular structures are understood and known by persons skilled in the art. Likewise, carbon atoms are not always specifically abbreviated with “C”, as the presence and location of carbon atoms in structural diagrams are known and understood by persons skilled in the art. Minutes are commonly abbreviated as “min”; hours are commonly abbreviated as “hr” or “h”.

A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or which “consists of”) the named elements or steps to the exclusion of any other unnamed element or step. In any composition or method disclosed herein, known or disclosed equivalents of any named essential element or step may be substituted for that element or step. It is also understood that an element or step “selected from the group consisting of” refers to one or more of the elements or steps in the list that follows, including combinations of any two or more of the listed elements or steps.

“Halo” or “halogen” as used herein means fluorine, chlorine, bromine, or iodine.

“Alkyl” means a straight or branched chain monovalent or divalent radical of saturated and/or unsaturated carbon atoms and hydrogen atoms, such as methyl (Me), ethyl (Et), propyl (Pr), isopropyl (iPr), butyl (Bu), isobutyl (iBu), sec-butyl (sBu), tert-butyl (tBu), and the like, which may be unsubstituted, or substituted by one or more suitable substituents found herein.

“Haloalkyl” means an alkyl moiety that is substituted with one or more identical or different halogen atoms, e.g., —CH₂Cl, —CF₃, —CH₂CF₃, —CH₂CCl₃, and the like.

“Alkenyl” means a straight-chain, branched, or cyclic hydrocarbon radical having from between 2-8 carbon atoms and at least one double bond, e.g., ethenyl, 3-buten-1-yl, 3-hexen-1-yl, cyclopent-1-en-3-yl, and the like, which may be unsubstituted, or substituted by one or more suitable substituents found herein.

“Alkynyl” means a straight-chain or branched hydrocarbon radical having from between 2-8 carbon atoms an at least one triple bond, e.g., ethynyl, 3-butyn-1-yl, 2-butyn-1-yl, 3-pentyn-1-yl, and the like, which may be unsubstituted, or substituted by one or more suitable substituents found herein.

“Cycloalkyl” as used herein means a non-aromatic monovalent or divalent monocyclic or polycyclic radical having from between 3-12 carbon atoms, each of which may be saturated or unsaturated, e.g., cyclopentyl, cyclohexyl, decalinyl, and the like, unsubstituted, or substituted by one or more of the suitable substituents found herein, and to which may be fused one or more aryl groups, heteroaryl groups, or heterocycloalkyl groups, which themselves may be unsubstituted or substituted by one or more suitable substituents found herein.

“Heterocycloalkyl” means a non-aromatic monovalent or divalent, monocyclic or polycyclic radical having from between 2-12 carbon atoms, and between 1-5 heteroatoms selected from nitrogen, oxygen, or sulfur, each of which may be saturated or unsaturated, e.g., pyrrolodinyl, tetrahydropyranyl, morpholinyl, piperazinyl, oxiranyl, and the like, unsubstituted, or substituted by one or more of the suitable substituents found herein, and to which may be fused one or more aryl groups, heteroaryl groups, or heterocycloalkyl groups, which themselves may be unsubstituted or substituted by one or more suitable substituents found herein.

“Aryl” means an aromatic monovalent or divalent monocyclic or polycyclic radical comprising between 6 and 18 carbon ring members, e.g., phenyl, biphenyl, naphthyl, phenanthryl, and the like, which may be substituted by one or more of the suitable substituents found herein, and to which may be fused one or more heteroaryl groups or heterocycloalkyl groups, which themselves may be unsubstituted or substituted by one or more suitable substituents found herein.

“Heteroaryl” means an aromatic monovalent or divalent monocyclic or polycyclic radical comprising between 6 and 18 ring members and at least one nitrogen heteroatom, e.g., pyridyl, pyrazinyl, pyridizinyl, pyrimidinyl, quinolinyl, and the like, which may be substituted by one or more of the suitable substituents found herein, and to which may be fused one or more aryl, heteroaryl groups or heterocycloalkyl groups, which themselves may be unsubstituted or substituted by one or more suitable substituents found herein.

“Hydroxy” means mean the radical —OH.

“Alkoxy” means the radical —OR where R is an alkyl or cycloalkyl group.

“Aryloxy” means the radical —OAr where Ar is an aryl group.

“Heteroaryloxy” means the radical —O(HAr) where HAr is a heteroaryl group.

“Acyl” means a —C(O)R radical where R is alkyl, alkenyl, alkynyl, cycloalkyl, aryl, heteroaryl, or heterocycloalkyl, e.g. acetyl, benzoyl, and the like.

“Carboxy” means the radical —C(O)OH.

“Alkoxycarbonyl” means a —C(O)OR radical where R is alkyl, alkenyl, alkynyl, or cycloalkyl.

“Aryloxycarbonyl” means a —C(O)OR radical where R is aryl or heteroaryl.

“Amino” means the radical —NH₂.

“Alkylamino” means the radical —NRR′ where R, and R′ are, independently, hydrogen, alkyl, alkenyl, alkynyl, cycloalkyl, aryl, or heteroaryl, or heterocycloalkyl.

“Acylamino” means the radical —NHC(O)R, where R is alkyl, alkenyl, alkynyl, cycloalkyl, aryl, heteroaryl, or heterocycloalkyl, e.g. acetyl, benzoyl, and the like, e.g., acetylamino, benzoylamino, and the like.

“Carboxamido” means the radical —C(O)NRR′ where R and R′ are, independently, hydrogen, alkyl, alkenyl, alkynyl, cycloalkyl, aryl, or heteroaryl, or heterocycloalkyl.

“Sulfonylamino” means the radical —NHSO₂R where R is alkyl, alkenyl, alkynyl, cycloalkyl, aryl, heteroaryl, or heterocycloalkyl.

“Amidino” means the radical —C(NR)NR′R″, where R, R′, and R″ are, independently, hydrogen, alkyl, alkenyl, alkynyl, cycloalkyl, aryl, or heteroaryl, and wherein R, R′, and R″ may form heterocycloalkyl rings, e.g., imidazolinyl, tetrahydropyrimidinyl.

“Guanidino” means the radical —NHC(NR)NR′R″, where R, R′, and R″ are, independently, hydrogen, alkyl, alkenyl, alkynyl, cycloalkyl, aryl, or heteroaryl, and wherein R, R′, and R″ may form heterocycloalkyl rings.

“Mercapto” means the radical —SH.

“Alkylthio” means the radical —SR where R is an alkyl or cycloalkyl group.

“Arylthio” means the radical —SAr where Ar is an aryl group.

“Hydroxamate” means the radical —C(O)NHOR where R is an alkyl or cycloalkyl group.

“Thioacyl” means a —C(S)R radical where R is alkyl, alkenyl, alkynyl, cycloalkyl, aryl, heteroaryl, or heterocycloalkyl.

“Alkylsulfonyl” means the radical —SO₂R where R is alkyl, alkenyl, alkynyl, cycloalkyl, aryl, heteroaryl, or heterocycloalkyl.

“Aminosulfonyl” means the radical —SO₂NRR′ where R and R′ are, independently, hydrogen, alkyl, alkenyl, alkynyl, cycloalkyl, aryl, or heteroaryl, or heterocycloalkyl.

The terms “bacterial type III secretion system inhibitor”, “bacterial T3SS inhibitor”, “bacterial T3SS inhibitor compound”, and “T3SS inhibitor compound” as used herein are interchangeable and denote compounds exhibiting the ability to specifically inhibit a bacterial type III secretion system by at least 15% at a concentration of 50 μM, for example, as measured in a T3SS effector transcriptional reporter assay or the ability to inhibit a bacterial T3SS, for example, as measured in a T3SS-mediated effector toxin secretion assay.

In the context of therapeutic use of the T3SS inhibitor compounds described herein, the terms “treatment”, “to treat”, or “treating” will refer to any use of the T3SS inhibitor compounds calculated or intended to arrest or inhibit the virulence or the T3SS-mediated effector secretion or translocation of bacteria having type III secretion systems. Thus, treating an individual may be carried out after any diagnosis indicating possible bacterial infection, i.e., whether an infection by a particular bacterium has been confirmed or whether the possibility of infection is only suspected, for example, after exposure to the bacterium or to another individual infected by the bacterium. It is also recognized that while the inhibitors of the present invention affect the introduction of effector toxins into host cells, and thus block or decrease the virulence or toxicity resulting from infection, the inhibitor compounds are not necessarily bactericidal or effective to inhibit growth or propagation of bacterial cells. For this reason, it will be understood that elimination of the bacterial infection will be accomplished by the host's own immune system or immune effector cells, or by introduction of antibiotic agents. Thus, it is contemplated that the compounds of the present invention will be routinely combined with other active ingredients such as antibiotics, antibodies, antiviral agents, anticancer agents, analgesics (e.g., a nonsteroidal anti-inflammatory drug (NSAID), acetaminophen, opioids, COX-2 inhibitors), immunostimulatory agents (e.g., cytokines or a synthetic immunostimulatory organic molecules), hormones (natural, synthetic, or semi-synthetic), central nervous system (CNS) stimulants, antiemetic agents, anti-histamines, erythropoietin, agents that activate complement, sedatives, muscle relaxants, anesthetic agents, anticonvulsive agents, antidepressants, antipsychotic agents, and combinations thereof.

The meaning of other terms will be understood by the context as understood by the skilled practitioner in the art, including the fields of organic chemistry, pharmacology, and microbiology.

The invention provides specific organic compounds that inhibit the T3SS of Pseudomonas aeruginosa. Structural analogs of previously studied T3SS inhibitors were evaluated for inhibition of T3SS-mediated secretion of an effector toxin-β-lactamase fusion protein (ExoS′-βLA) using P. aeruginosa strain MDM973 (PAK/pUCP24GW-lacI^{Q}-lacPO-exoS::blaM, Table 1). See, Examples 1 and 2, below for details of screening and validation of initial T3SS inhibitors.

In a series of experiments to compare the effects of modifying the phenoxyacetamide scaffold, of which compound MBX-1641 is a prototypical example,

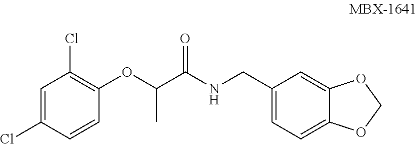 [SMILES: CC(Oc1ccc(Cl)cc1Cl)C(=O)NCc1ccc2c(c1)OCO2]

analogs were synthesized having alterations to the “A” aryl group, to the linker of the A aryl group, to the methyl acetamide moiety, to the “B” aryl group, and to the linker of the B aryl group (see Diagram 1) and the results indicated defined limitations to alternate structures on the methyl acetamide scaffold that would yield compounds also having specific inhibitory activity with respect to T3SS.
Very few modifications of the A aryl group could be tolerated without raising inhibitory concentration levels (IC₅₀) beyond the minimal standard (i.e., 200 μM); however, introduction of third substituent onto the A aryl group improved IC₅₀ (>5 fold)). Alternate linker moieties to the A aryl group and the B aryl group were studied, and those positions were found to exhibit a significant influence on overall properties of the resulting compounds. Similarly, changes to eliminate the methyl group at the chiral center (α carbon) or to increase the size of the substituent group also led to significant effects on T3SS inhibitory properties. A wide range of substitutions for the B aryl group have proven extremely promising, with excellent in vitro potency as well as ADME properties; specially zwitterionic groups represent a privileged motif, necessary for both potency and microsomal stability. In general, the structure/activity relationships emerging from the experiments were characteristic of discoveries respecting alternative compounds reactive with a single target binding site.

From the program of analog synthesis and comparative testing a family of new compounds emerged which exhibited T3SS inhibitory properties comparable to and in many cases greater than the phenoxyacetamide inhibitor compounds that had been described previously. The family of new T3SS inhibitor compounds is defined by formula I:

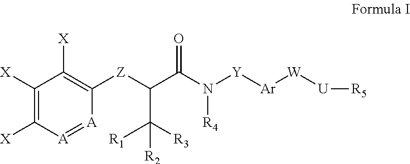

Wherein,
A is independently selected from CH or N;
X is independently selected from hydrogen, halogen or hydroxyl;
Z is O, S, NH; or NR′, wherein R′ is alkyl;
R₁, R₂, and R₃ are independently selected from: hydrogen, halogen, alkyl, hydroxy, alkoxy, alkylthio, or cyano, wherein no more than two of the preceding radicals is hydrogen; NR₄ wherein;
R₄ is hydrogen, a straight chain aliphatic group, a branched chain aliphatic group, cycloalkyl, haloalkyl, hydroxyalkyl, alkoxy, alkylamino, alkylthio, sulfonyl, sulfinyl, carboxy, alkoxycarbonyl or an aryl;
Y is selected from:
a divalent straight-chain, branched, or cyclic alkyl, alkenyl or alkynyl radical of from 1 to 6 carbon atoms, which may contain one or more heteroatoms, and which may be unsubstituted or substituted with up to four substituents selected from halo, cyano, hydroxy, amino, alkyl, cycloalkyl, alkylamino, carboxyl, alkoxycarbonyl, carboxamido, acylamino, amidino, sulfonamide, aminosulfonyl, alkylsulfonyl, aryl, heteroaryl, alkoxy, alkylthio; aryloxy, and heteroaryloxy; oxygen; oxime; or NR″ where R″ is hydrogen, alkyl or cycloalkyl;
Ar is an aryl or heteroaryl radical forming a five-membered or six-membered ring which may be additionally fused with from 1 to 3 aryl, heteroaryl, cycloalkyl, or heterocycloalkyl rings, which Ar radical may be unsubstituted or substituted with up to four substituents selected from halo, cyano, hydroxy, amino, alkyl, cycloalkyl, alkylamino, carboxyl, alkoxycarbonyl, carboxamido, acylamino, amidino, sulfonamido, aminosulfonyl, alkylsulfonyl, aryl, heteroaryl, alkoxy, alkylthio; aryloxy, and heteroaryloxy, and wherein any two substituents together may form an aromatic or non-aromatic ring structure fused with said aryl or heteroaryl radical Ar, or alternatively wherein substituents on Ar also may be optionally bonded covalently to either Y or R₄, or both Y and R₄, to form heterocyclic or carbocyclic ring systems, which ring systems may be aromatic, heteroaromatic, or partly aromatic, (that is, one or more rings being aromatic and one or more rings being non-aromatic (saturated));
W is a divalent radical bridging Ar and U selected from the group comprising, —COCH₂—, —SO₂—, —NHSO₂—, —SO2NH—, —CO—, —CH₂—, —CH(CH₃)—, —NHCO—, —NHCONH— —NCH₃CO—, —CONH—, —CONCH₃—, —O(CO)—, —(CO)O—, —NH—, or —O—;
U can be either a nitrogen or a carbon that is part of a non-aromatic heterocyclic ring of from 4-7 members and containing 1-2 nitrogen atoms, 0-1 oxygen atoms and bearing 0-3 substituents (in addition to the bond between W and the ring carbon/nitrogen and the U and the ring carbon in the formula) selected from alkyl, cycloalkyl, aryl, heteroaryl, haloalkyl, nitro, halogen, alkoxy, alkylthio, haloalkoxy, sulfonyl, or sulfinyl; and additionally U can be either a nitrogen or a saturated carbon that is linked via a chain of 1-4 carbon atoms to a basic nitrogen bearing, independently, hydrogen or aliphatic groups of less than nine carbon atoms that can be optionally substituted with groups selected from haloalkyl, nitro, halogen, alkoxy, alkylthio, haloalkoxy, sulfonyl, sulfinyl, carboxy, alkoxycarbonyl, or aminocarbonyl groups, heteroaryl, haloalkyl, nitro, halogen, alkoxy, alkylthio, haloalkoxy, sulfonyl, or sulfinyl;
R₅ is a monovalent radical selected from the group comprising, —CH2C(CH3)2COOH, —CH2COOH, —CH2COOH, —CH2CH2CH2COOH, —(CyBut)COOH, —CH2(CyPropyl)COOH, CH2(CyPropyl)COOH, —SO₂Me, —SO₂OH, —SO₂CF₃

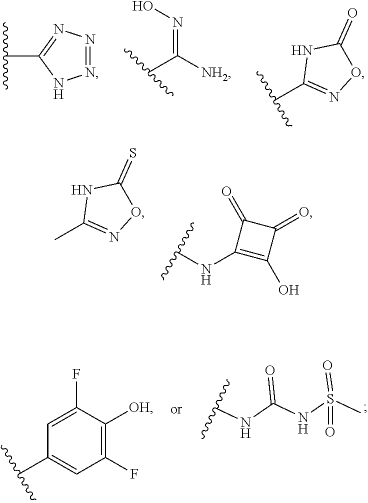

In another embodiment, the present invention provides a family of bacterial type III secretion system (T3SS) inhibitor compounds having the structure of Formula I(a):

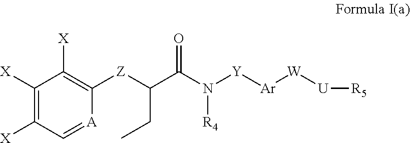

A is independently selected from CH or N;
X is independently selected from hydrogen or halogen;
Z is O, S, NH; or NR′, wherein R′ is alkyl;
R₄ is hydrogen or methyl;
Y is selected from:
a divalent straight-chain, branched, or cyclic alkyl, alkenyl or alkynyl radical of from 1 to 6 carbon atoms, which may contain one or more heteroatoms, and which may be unsubstituted or substituted with up to four substituents selected from halo, cyano, hydroxy, amino, alkyl, cycloalkyl, alkylamino, carboxyl, alkoxycarbonyl, carboxamido, acylamino, amidino, sulfonamide, aminosulfonyl, alkylsulfonyl, aryl, heteroaryl, alkoxy, alkylthio; aryloxy, and heteroaryloxy; oxygen; oxime; or NR″ where R″ is hydrogen, alkyl or cycloalkyl;
Ar is an aryl or heteroaryl divalent radical forming a five-membered or six-membered ring which may be additionally fused with from 1 to 3 aryl, heteroaryl, cycloalkyl, or heterocycloalkyl rings, which Ar radical may be unsubstituted or substituted with up to four substituents selected from halo, cyano, hydroxy, amino, alkyl, cycloalkyl, alkylamino, carboxyl, alkoxycarbonyl, carboxamido, acylamino, amidino, sulfonamido, aminosulfonyl, alkylsulfonyl, aryl, heteroaryl, alkoxy, alkylthio; aryloxy, and heteroaryloxy, and wherein any two substituents together may form an aromatic or non-aromatic ring structure fused with said aryl or heteroaryl radical Ar, or alternatively wherein substituents on Ar also may be optionally bonded covalently to either Y or R₄, or both Y and R₄, to form heterocyclic or carbocyclic ring systems, which ring systems may be aromatic, heteroaromatic, or partly aromatic, (that is, one or more rings being aromatic and one or more rings being non-aromatic (saturated)).
W is a divalent radical bridging Ar and U selected from the group comprising, —COCH₂—, —SO₂—, —NHSO₂—, —SO2NH—, —CO—, —CH₂—, —CH(CH₃)—, —NHCO—, —NHCONH— —NCH₃CO—, —CONH—, —CONCH₃—, —O(CO)—, —(CO)O—, —NH—, or —O—;
U can be either a nitrogen or a carbon that is part of a non-aromatic heterocyclic ring system of between 4-7 members and containing 1-2 nitrogen atoms, 0-1 oxygen atoms and bearing 0-3 substituents (in addition to the bond between W and the ring carbon/nitrogen and the U and the ring carbon in the formula) selected from alkyl, cycloalkyl, aryl, heteroaryl, haloalkyl, nitro, halogen, alkoxy, alkylthio, haloalkoxy, sulfonyl, or sulfinyl; and additionally W can be either a nitrogen or a saturated carbon that is linked via a chain of 1-4 carbon atoms to a basic nitrogen bearing, independently, hydrogen or aliphatic groups of less than nine carbon atoms that can be optionally substituted with groups selected from haloalkyl, nitro, halogen, alkoxy, alkylthio, haloalkoxy, sulfonyl, sulfinyl, carboxy, alkoxycarbonyl, or aminocarbonyl groups, heteroaryl, haloalkyl, nitro, halogen, alkoxy, alkylthio, haloalkoxy, sulfonyl, or sulfinyl;
R₅ is a monovalent radical selected from the group comprising, —CH2C(CH3)2COOH, —CH2COOH, —CH2COOH, —CH2CH2CH2COOH, —(CyBut)COOH, —CH2(CyPropyl)COOH, CH2(CyPropyl)COOH, —SO₂Me, —SO₂OH, —SO₂CF₃

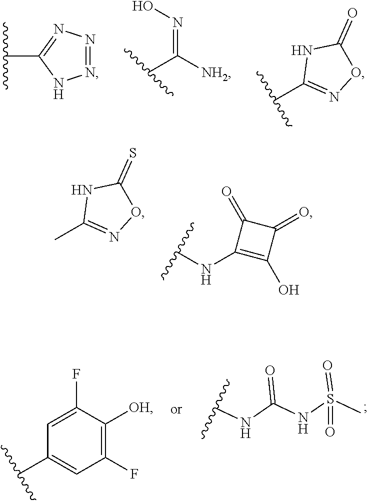

In another embodiment, the present invention provides a family of bacterial type III secretion system (T3SS) inhibitor compounds having the structure of Formula I(b):

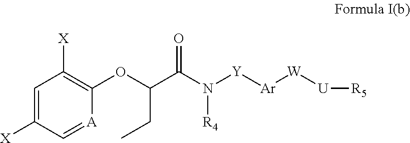

A is CH or N;
X is independently selected from hydrogen or halogen;
Y is —CH₂—, —CH(CH₃)—, or —C(CH₃)₂—; and
Ar is an aryl or heteroaryl divalent radical forming a five-membered or six-membered ring which may be additionally fused with from 1 to 3 aryl, heteroaryl, cycloalkyl, or heterocycloalkyl rings, which Ar radical may be unsubstituted or substituted with up to four substituents selected from halo, cyano, hydroxy, amino, alkyl, cycloalkyl, alkylamino, carboxyl, alkoxycarbonyl, carboxamido, acylamino, amidino, sulfonamido, aminosulfonyl, alkylsulfonyl, aryl, heteroaryl, alkoxy, alkylthio; aryloxy, and heteroaryloxy, and wherein any two substituents together may form an aromatic or non-aromatic ring structure fused with said aryl or heteroaryl radical Ar, or alternatively wherein substituents on Ar also may be optionally bonded covalently to Y; to form heterocyclic or carbocyclic ring systems, which ring systems may be aromatic, heteroaromatic, or partly aromatic, (that is, one or more rings being aromatic and one or more rings being non-aromatic (saturated)).
W is a divalent radical bridging Ar and U selected from the group comprising, —COCH₂—, —SO₂—, —NHSO₂—, —SO2NH—, —CO—, —CH₂—, —CH(CH₃)—, —NHCO—, —NHCONH— —NCH₃CO—, —CONH—, —CONCH₃—, —O(CO)—, —(CO)O—, —NH—, or —O—;
U can be either a nitrogen or a carbon that is part of a non-aromatic heterocyclic ring system of between 4-7 members and containing 1-2 nitrogen atoms, 0-1 oxygen atoms and bearing 0-3 substituents (in addition to the bond between W and the ring carbon/nitrogen and the U and the ring carbon in the formula) selected from alkyl, cycloalkyl, aryl, heteroaryl, haloalkyl, nitro, halogen, alkoxy, alkylthio, haloalkoxy, sulfonyl, or sulfinyl; and additionally W can be either a nitrogen or a saturated carbon that is linked via a chain of 1-4 carbon atoms to a basic nitrogen bearing, independently, hydrogen or aliphatic groups of less than nine carbon atoms that can be optionally substituted with groups selected from haloalkyl, nitro, halogen, alkoxy, alkylthio, haloalkoxy, sulfonyl, sulfinyl, carboxy, alkoxycarbonyl, or aminocarbonyl groups, heteroaryl, haloalkyl, nitro, halogen, alkoxy, alkylthio, haloalkoxy, sulfonyl, or sulfinyl;
R₅ is a monovalent radical selected from the group comprising, —CH2C(CH3)2COOH, —CH2COOH, —CH2COOH, —CH2CH2CH2COOH, —(CyBut)COOH, —CH2(CyPropyl)COOH, CH2(CyPropyl)COOH, —SO₂Me, —SO₂OH, —SO₂CF₃

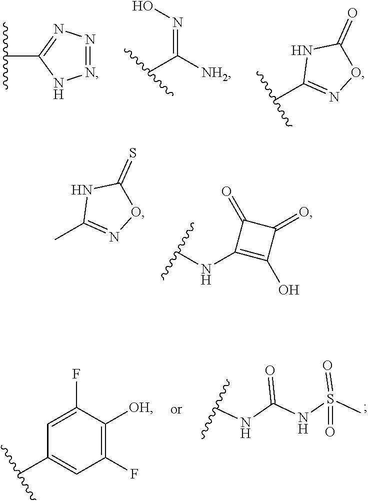

In another embodiment, the present invention provides a family of bacterial type III secretion system (T3SS) inhibitor compounds having the structure of Formula I(c):

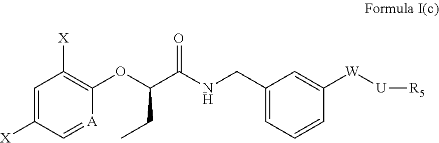

A is CH or N;
at least one X is Cl and the other X is hydrogen, F, or Cl;
W is a divalent radical bridging Ar and U selected from the group comprising, —COCH₂—, —SO₂—, —NHSO₂—, —SO2NH—, —CO—, —CH₂—, —CH(CH₃)—, —NHCO—, —NHCONH— —NCH₃CO—, —CONH—, —CONCH₃—, —O(CO)—, —(CO)O—, —NH—, or —O—;
U can be either a nitrogen or a carbon that is part of a non-aromatic heterocyclic ring system of between 4-7 members and containing 1-2 nitrogen atoms, 0-1 oxygen atoms and bearing 0-3 substituents (in addition to the bond between W and the ring carbon/nitrogen and the U and the ring carbon in the formula) selected from alkyl, cycloalkyl, aryl, heteroaryl, haloalkyl, nitro, halogen, alkoxy, alkylthio, haloalkoxy, sulfonyl, or sulfinyl; and additionally W can be either a nitrogen or a saturated carbon that is linked via a chain of 1-4 carbon atoms to a basic nitrogen bearing, independently, hydrogen or aliphatic groups of less than nine carbon atoms that can be optionally substituted with groups selected from haloalkyl, nitro, halogen, alkoxy, alkylthio, haloalkoxy, sulfonyl, sulfinyl, carboxy, alkoxycarbonyl, or aminocarbonyl groups, heteroaryl, haloalkyl, nitro, halogen, alkoxy, alkylthio, haloalkoxy, sulfonyl, or sulfinyl;
R₅ is a monovalent radical selected from the group comprising, —CH2C(CH3)2COOH, —CH2COOH, —CH2COOH, —CH2CH2CH2COOH, —(CyBut)COOH, —CH2(CyPropyl)COOH, CH2(CyPropyl)COOH, —SO₂Me, —SO₂OH, —SO₂CF₃

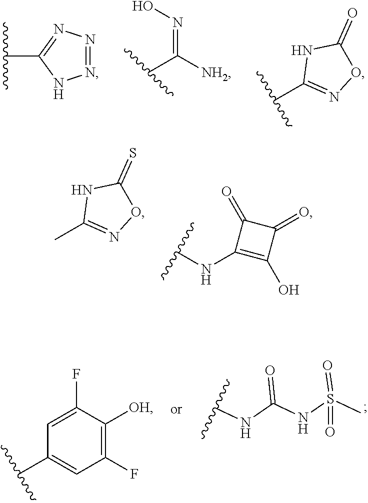

Additional embodiments of the present invention that are not encompassed by Formula I, Formula II, or Formula III above include compounds of Table 2:

| Compound No. | |
|---|---|
| MBX- | Structure |
| 5050 | 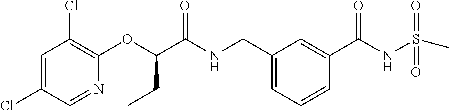 [SMILES: CC[C@@H](Oc1ncc(Cl)cc1Cl)C(=O)NCc1cccc(C(=O)NS(C)(=O)=O)c1] |
| 5414 | 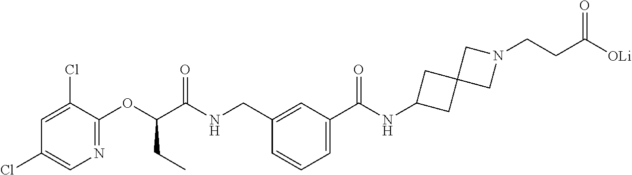 |
| 5427 | 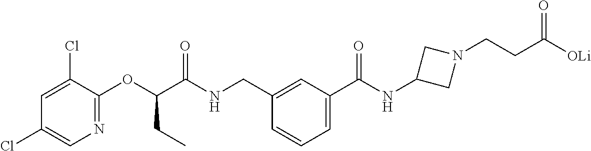 |
| 5430 | 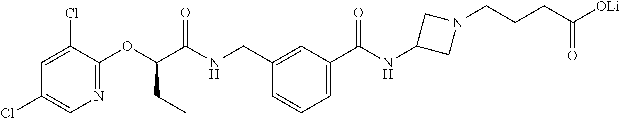 |
| 5442 | 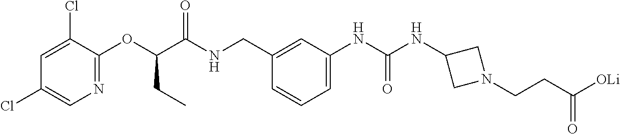 |
| 5452 | 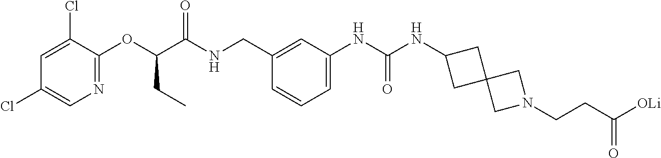 |
| 5453 | 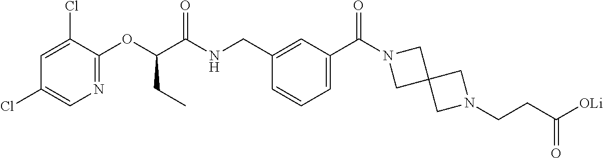 |
| 5454 | 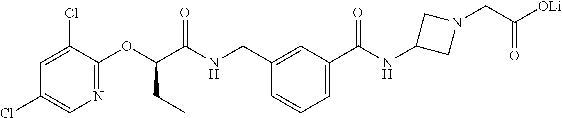 |
| 5456 | 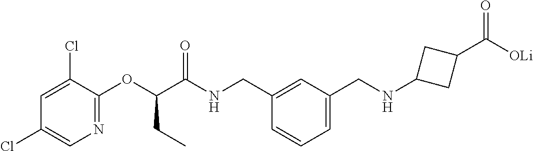 |
| 5499 | 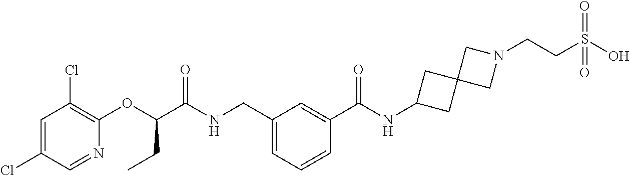 [SMILES: CC[C@@H](Oc1ncc(Cl)cc1Cl)C(=O)NCc1cccc(C(=O)NC2CC3(C2)CN(CCS(=O)(=O)O)C3)c1] |
| 5508 | 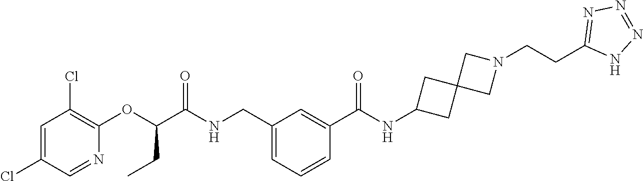 [SMILES: CC[C@@H](Oc1ncc(Cl)cc1Cl)C(=O)NCc1cccc(C(=O)NC2CC3(C2)CN(CCc2nnn[nH]2)C3)c1] |
| 5522 | 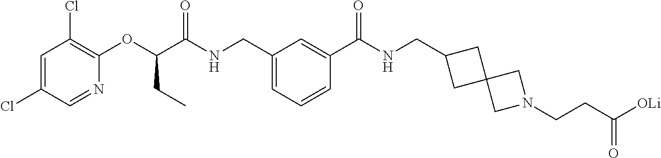 |
| 5523 | 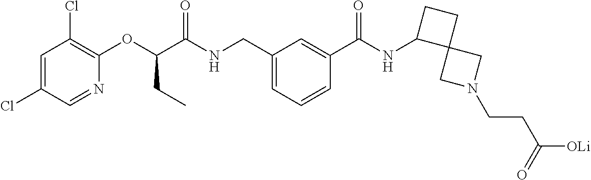 |
| 5524 | 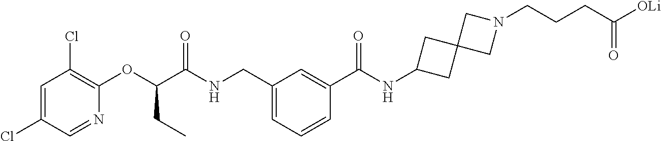 |
| 5525 | 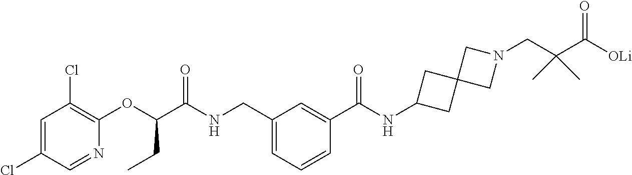 |
| 5526 | 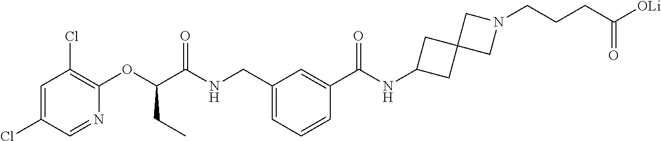 |
| 5527 | 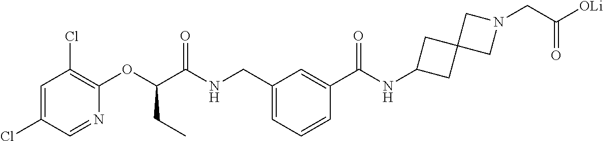 |
| 5529 | 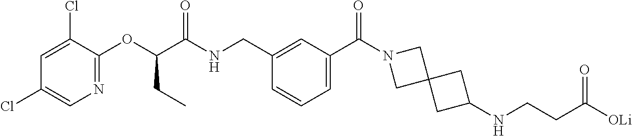 |
| 5530 | 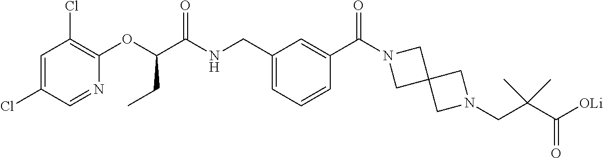 |
| 5531 | 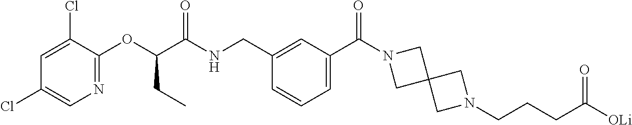 |
| 5532 | 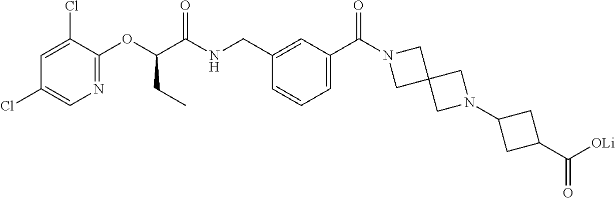 |
| 5533 | 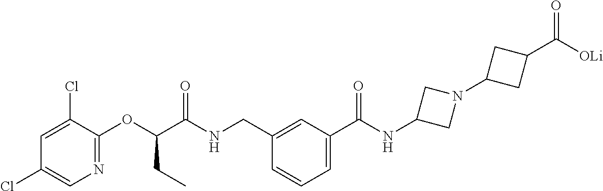 |
| 5536 | 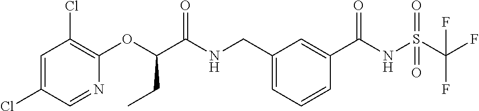 [SMILES: CC[C@@H](Oc1ncc(Cl)cc1Cl)C(=O)NCc1cccc(C(=O)NS(=O)(=O)C(F)(F)F)c1] |
| 5548 | 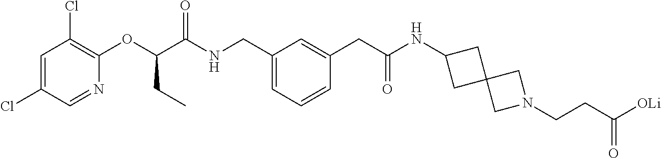 |
| 5586 | 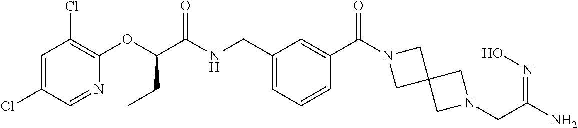 [SMILES: CC[C@@H](Oc1ncc(Cl)cc1Cl)C(=O)NCc1cccc(C(=O)N2CC3(CN(C/C(N)=N\O)C3)C2)c1] |
| 5590 | 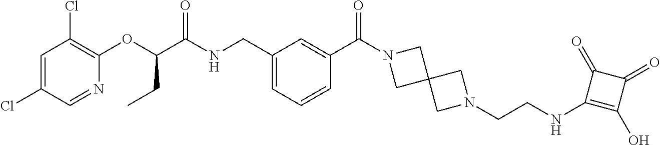 [SMILES: CC[C@@H](Oc1ncc(Cl)cc1Cl)C(=O)NCc1cccc(C(=O)N2CC3(CN(CCNc4c(O)c(=O)c4=O)C3)C2)c1] |
| 5591 | 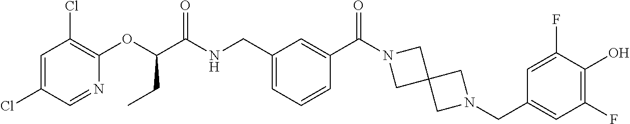 [SMILES: CC[C@@H](Oc1ncc(Cl)cc1Cl)C(=O)NCc1cccc(C(=O)N2CC3(CN(Cc4cc(F)c(O)c(F)c4)C3)C2)c1] |
| 5592 | 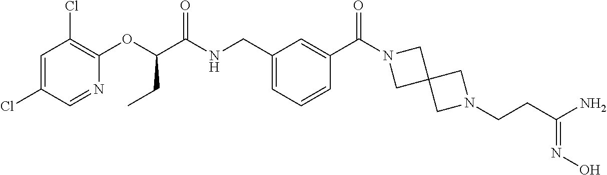 [SMILES: CC[C@@H](Oc1ncc(Cl)cc1Cl)C(=O)NCc1cccc(C(=O)N2CC3(CN(CC/C(N)=N/O)C3)C2)c1] |
| 5593 | 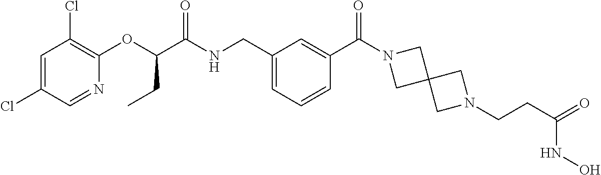 [SMILES: CC[C@@H](Oc1ncc(Cl)cc1Cl)C(=O)NCc1cccc(C(=O)N2CC3(CN(CCC(=O)NO)C3)C2)c1] |
| 5595 | 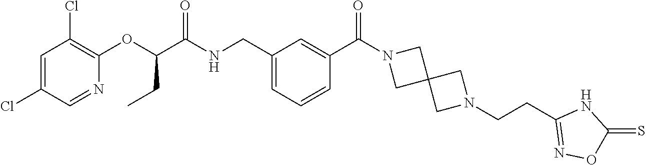 [SMILES: CC[C@@H](Oc1ncc(Cl)cc1Cl)C(=O)NCc1cccc(C(=O)N2CC3(CN(CCc4noc(=S)[nH]4)C3)C2)c1] |
| 5596 | 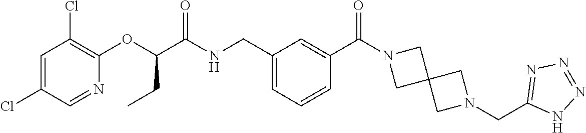 [SMILES: CC[C@@H](Oc1ncc(Cl)cc1Cl)C(=O)NCc1cccc(C(=O)N2CC3(CN(Cc4nnn[nH]4)C3)C2)c1] |
| 5597 | 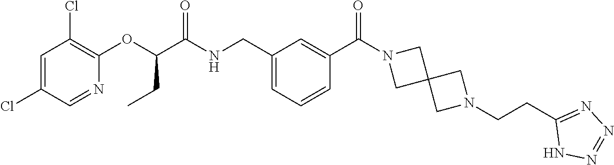 [SMILES: CC[C@@H](Oc1ncc(Cl)cc1Cl)C(=O)NCc1cccc(C(=O)N2CC3(CN(CCc4nnn[nH]4)C3)C2)c1] |
| 5598 | 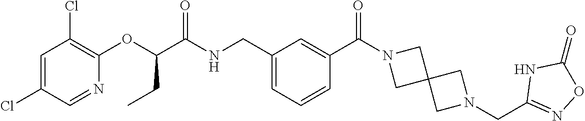 [SMILES: CC[C@@H](Oc1ncc(Cl)cc1Cl)C(=O)NCc1cccc(C(=O)N2CC3(CN(Cc4noc(=O)[nH]4)C3)C2)c1] |
| 5619 | 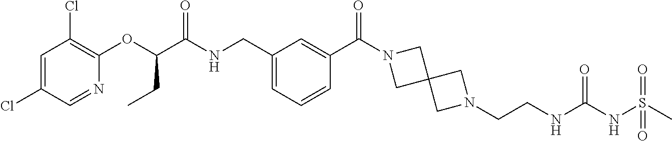 [SMILES: CC[C@@H](Oc1ncc(Cl)cc1Cl)C(=O)NCc1cccc(C(=O)N2CC3(CN(CCNC(=O)NS(C)(=O)=O)C3)C2)c1] |
| 6679 | 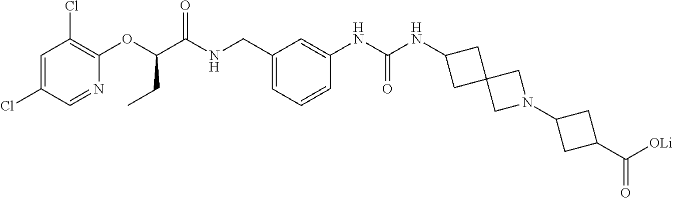 |
| 6680 | 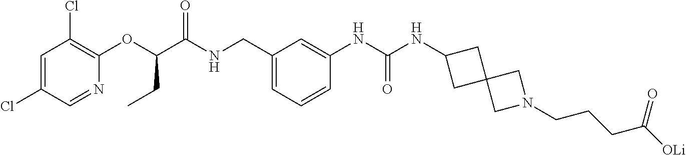 |
| 6681 | 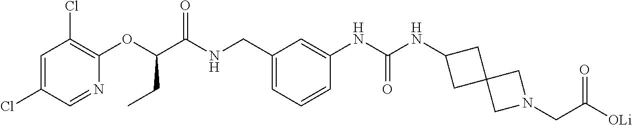 |
| 6754 | 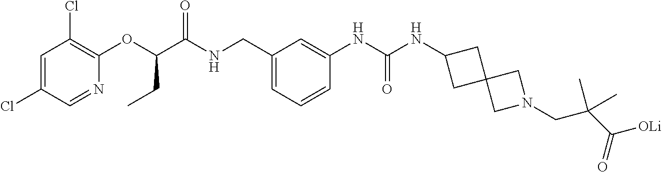 |
| 6948 | 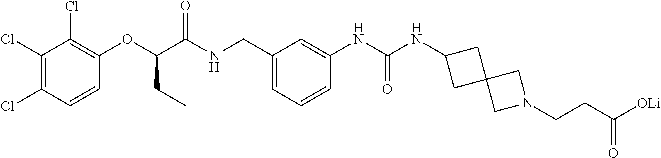 |
| 6949 | 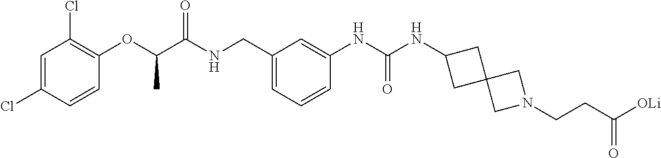 |
| 7061 | 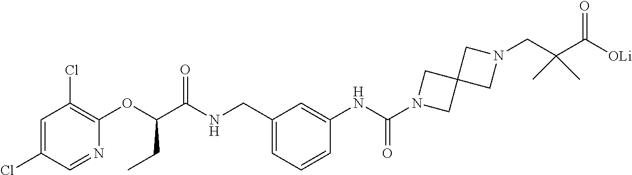 |
| 7062 | 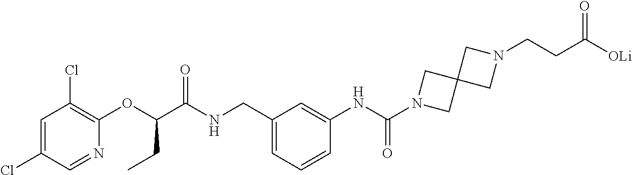 |
| 7119 | 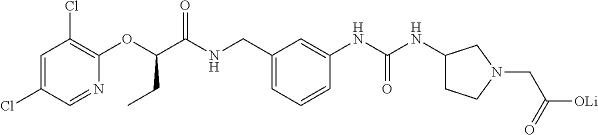 |
| 7120 | 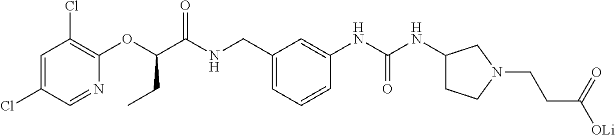 |
| 7121 | 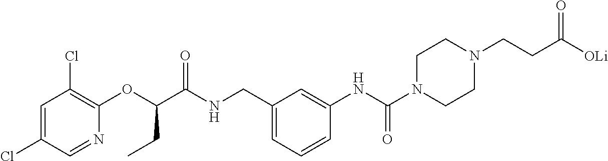 |
| 7140 | 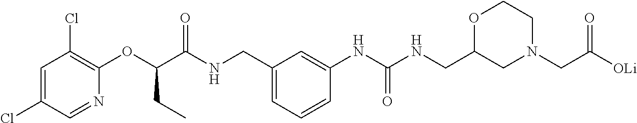 |
| 7141 | 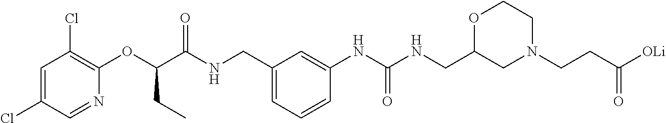 |
| 7142 | 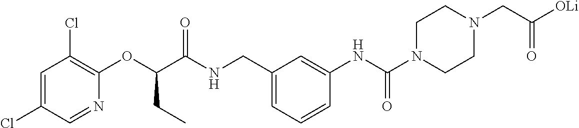 |
| 7143 | 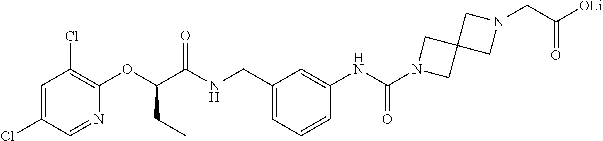 |
| 7145 | 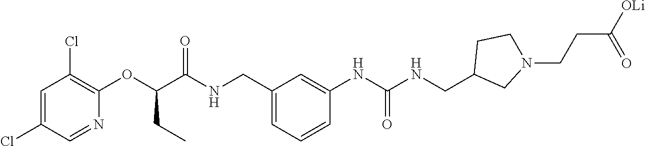 |
| 7146 | 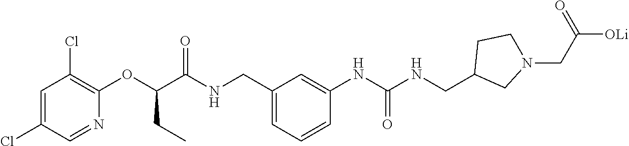 |
| 7211 | 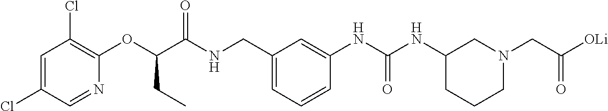 |
| 7212 | 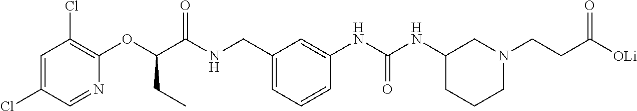 |
| 7290 | 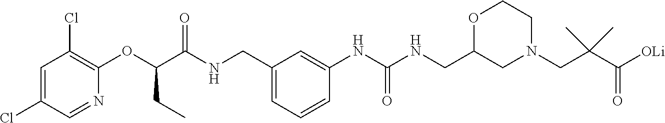 |
| 7291 | 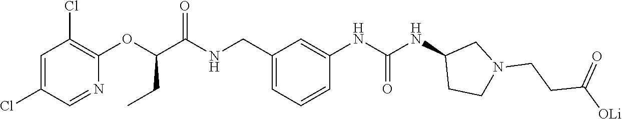 |
| 7292 | 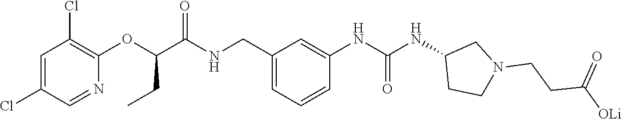 |
| 7293 | 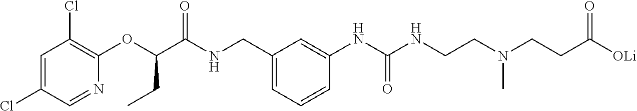 |
| 7303 | 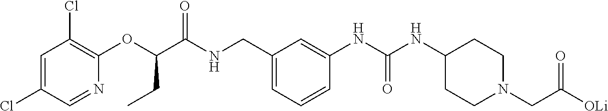 |
| 7304 | 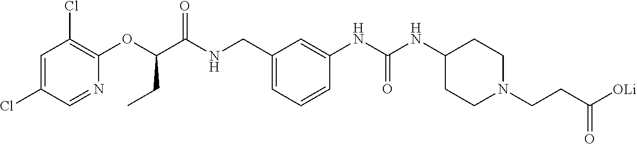 |
| 7305 | 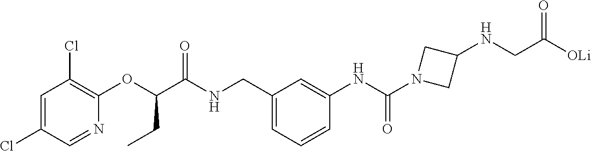 |
| 7306 | 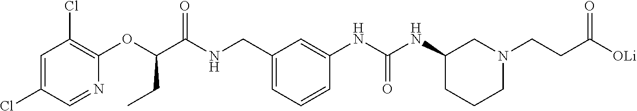 |
| 7307 | 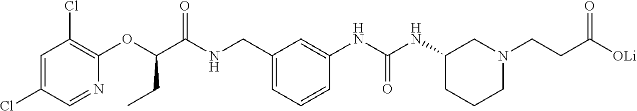 |
| 7326 | 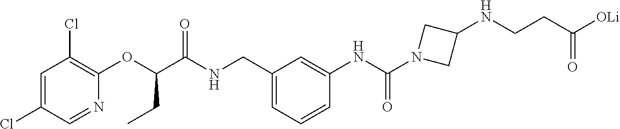 |
| 7339 | 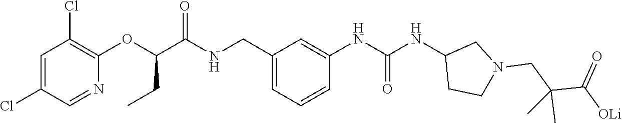 |
| 7340 | 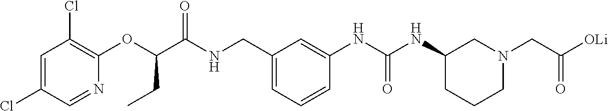 |
| 7341 | 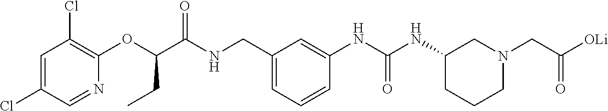 |
| 7352 | 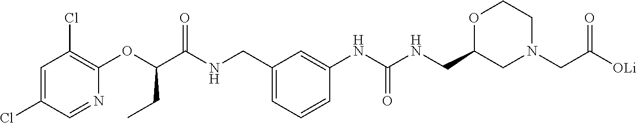 |
| 7353 | 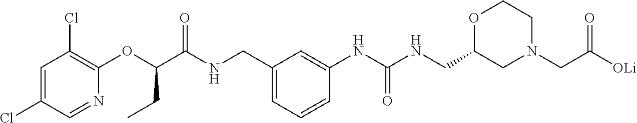 |
| 7388 | 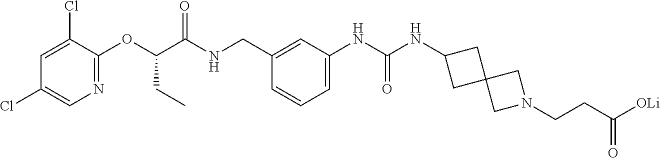 |
| 7393 | 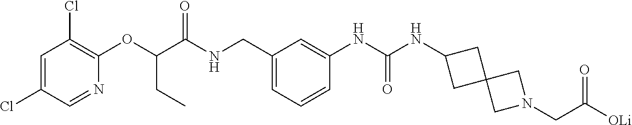 |
| 7394 | 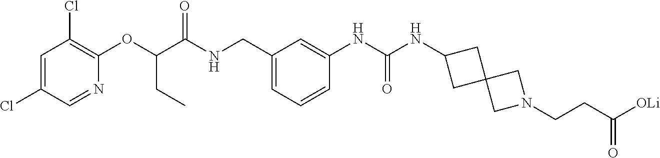 |
| 7419 | 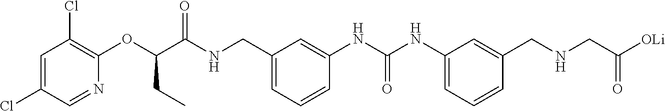 |
| 7420 | 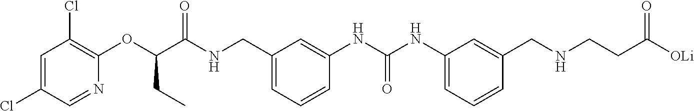 |
| 7421 | 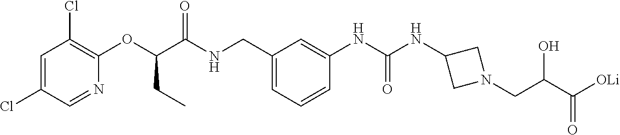 |
| 7422 | 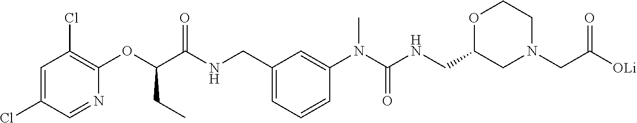 |
| 7423 | 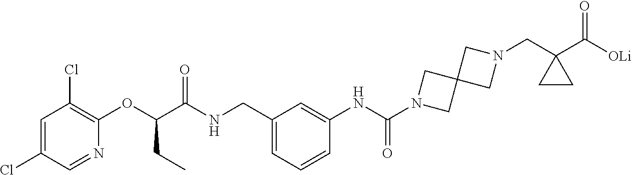 |
| 7424 | 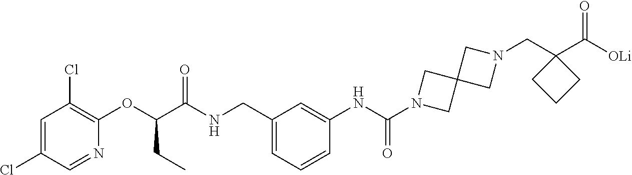 |
| 7434 | 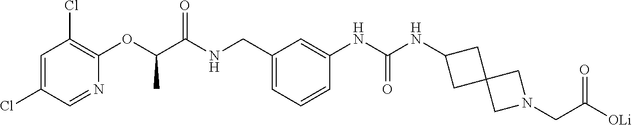 |
| 7435 | 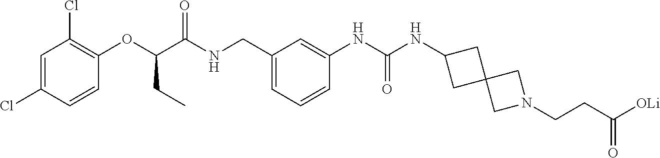 |
| 7436 | 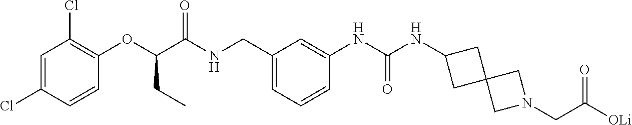 |
| 7437 | 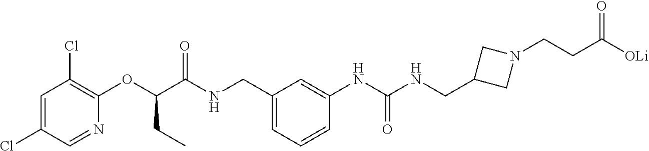 |
| 7438 | 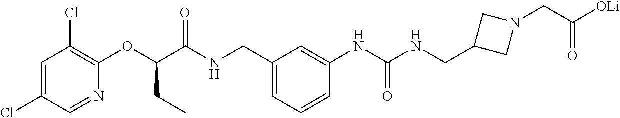 |
| 7439 | 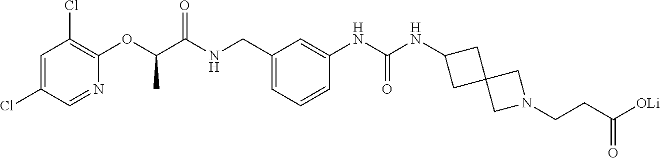 |
| 7463 | 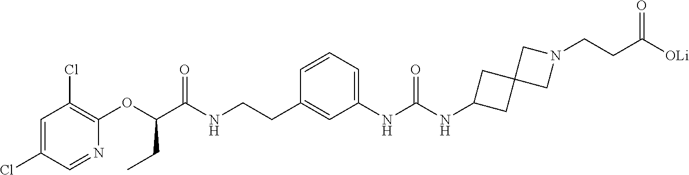 |
| 7464 | 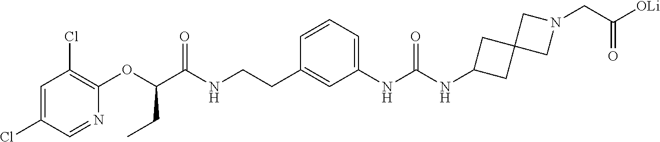 |
| 7476 | 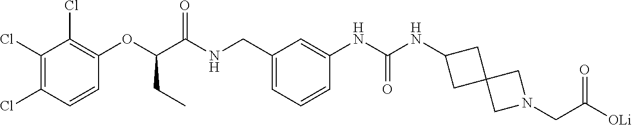 |
| 7477 | 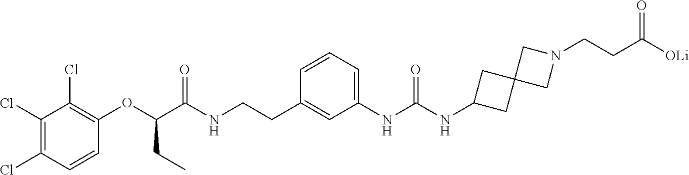 |
| 7478 | 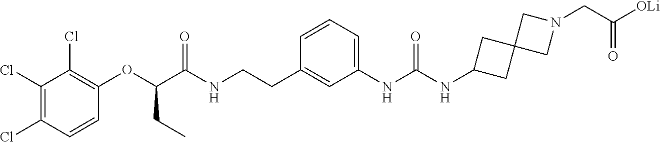 |
| 7495 | 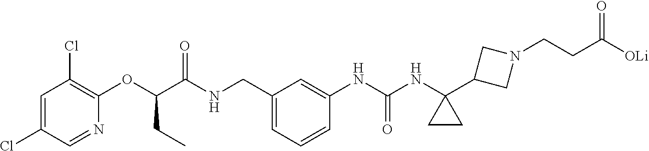 |
| 7496 | 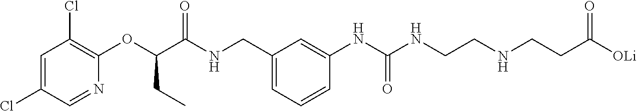 |
| 7497 | 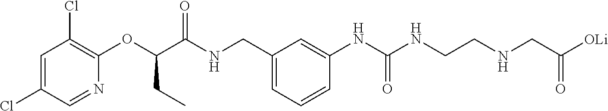 |
| 7531 | 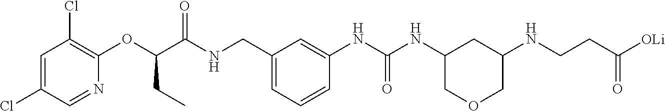 |

as well as particular isomeric forms of any of the foregoing.

The compounds of the present invention are designed to function by a novel anti-virulence approach of potentiating the activity of existing antibacterial agents by bolstering the host innate immune system rather than directly killing invading bacteria. While not classic innate immune modulators, these anti-T3SS agents are believed to act indirectly on host targets by protecting the phagocytes of the innate immune system from most of the acute cytotoxic effects of bacteria having type III secretion systems such as P. aeruginosa. As therapeutic agents, the compounds of the invention may reduce the frequency of polymicrobial VAP infections, which appear to be due to local innate immune suppression by P. aeruginosa T3SS effector toxins. (Diaz, et al., 2008, Pseudomonas aeruginosa induces localized immunosuppression during pneumonia, Infect. Immun., 76:4414-21.) Furthermore, these compounds of the present invention are species-specific and consequently spare normal flora, advantageously aligning this therapeutic approach with an emerging understanding of the protective role of the normal flora in infectious diseases. Parillo and Dellinger, Critical Care Medicine: Principles of Diagnosis and Management in the Adult, 2^{nd} ed. (Moseby, New York 2007), pp. 800-802. If applied in combination with an antibacterial agent, the new T3SS inhibitor compounds will not contribute to the elimination of normal flora and may permit the use of lower doses of co-administered antibiotics. Finally, these T3SS inhibitor compounds are equally potent against multiple P. aeruginosa strains (including clinical isolates), are not affected by P. aeruginosa efflux mechanisms, and are expected to exert no selection pressure for the development of resistance outside the body and only relatively weak selection pressure during therapy. This combination of favorable features of the compounds together with the novel mechanism of action provides a new approach to improve the treatment and prevention of acute P. aeruginosa infections such as VAP and bacteremia.

Inhibitor compounds of the present invention inhibit T3SS effector transcription by at least 15% at a concentration of 50 μM as demonstrated in a transcriptional reporter assay or by exhibiting at least 50% inhibition of effector secretion at a concentration of 100 M or less (IC₅₀≤100 μM) as demonstrated in an effector secretion assay. The compounds listed above showed T3SS-specific inhibition in Pseudomonas of greater than 15% using an exoT-lux transcriptional reporter construct transferred into Pseudomonas aeruginosa PAO1 (reporter strain MDM852, described herein) and/or showed an IC₅₀ value of less than 100 μM for T3SS as measured in an assay of T3SS-mediated secretion of an effector toxin-β-lactamase reporter fusion protein assay described herein using P. aeruginosa strain MDM973 (PAK/pUCP24GW-lacI^{Q}-lacPO-exoS::blaM) (Table 1).

In particularly preferred embodiments, a T3SS inhibitor compound useful in the compositions and methods described herein has an IC₅₀ value of less than 100 μM as measured in a T3SS-mediated effector toxin-β-lactamase reporter fusion protein secretion assay described herein (or comparable assay) and also has a relatively low cytotoxicity toward human cells, preferably a CC₅₀ value of greater than or equal to 100 μM (CC₅₀≥100 μM) as measured in a standard cytotoxicity assay as described herein or as employed in the pharmaceutical field for antibiotics. Such standard cytotoxicity assays may employ any human cell typically employed in cytotoxicity assays for antibiotics, including but not limited to, Chinese hamster ovary (CHO) cells, HeLa cells, Hep-2 cells, human embryonic kidney (HEK) 293 cells, 293T cells, and the like.

Even more preferably, a T3SS inhibitor compound described herein has an IC₅₀ value ≤25 μM as measured in a T3SS-mediated effector toxin-β-lactamase reporter fusion protein secretion assay as described herein or in a comparable assay. Alternatively, preferred compounds of the present invention exhibit potency (IC₅₀) comparable or preferably greater than that of N-(benzo[d][1,3]dioxol-5-ylmethyl)-2-(2,4-dichlorophenoxy)propanamide (compound MBX-1641, described supra), which was used as an internal standard for comparison in the examples described below.

In yet another embodiment, a T3SS inhibitor compound described herein has a sufficiently high minimal inhibitory concentration (MIC) to indicate that it inhibits T3SS specifically and has no effect on the viability of bacterial cells in culture in the absence of phagocytes.

In a particularly preferred embodiment of the invention, a T3SS inhibitor compound blocks T3SS-mediated secretion and translocation of one or more toxin effectors from cells of P. aeruginosa, and is safe and achieves sufficiently high levels and of sufficient duration in appropriate animal tissues to exhibit efficacy in animal models of infection such as the murine lung infection model.

Even more preferably, to be useful as a drug, a T3SS inhibitor compound described herein has a translocation inhibition EC₅₀<10 uM vs. 90% of P. aeruginosa clinical strains tested, is stable in human serum (>75% stable for >1 hr), is stable in human liver microsome preparations (t_{1/2}>1 hr), is soluble at concentrations >5 mg/mL in clinically acceptable formulations, and displays efficacy in vivo in an animal model of P. aeruginosa infection.

### Compositions and Methods

T3SS inhibitor compounds as described herein may also be synthesized using established chemistries.

The following schemes provide suitable synthesis schemes for the preferred embodiments.

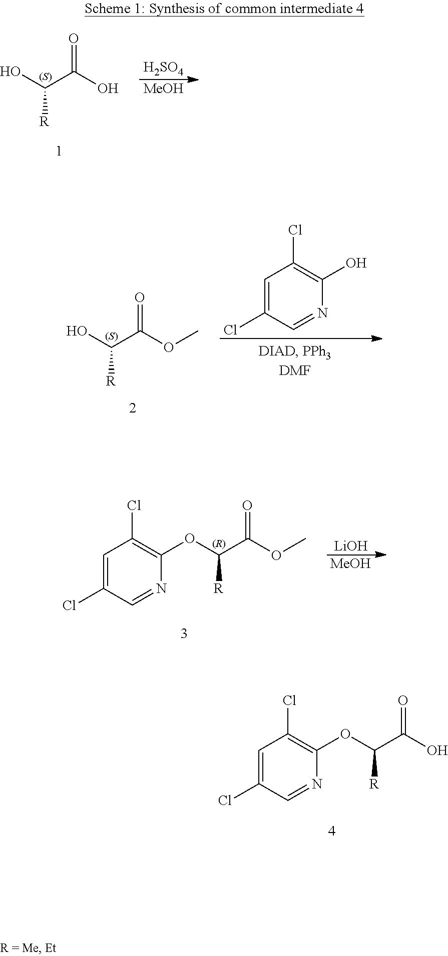

R=Ethyl: (S)-methyl 2-hydroxybutanoate (2): (S)-2-hydroxybutanoic acid (1) (24.47 g, 235.1 mmol) is dissolved in MeOH (1 L), to which H₂SO₄ (~0.5 mL) was added, and the mixture was heated to vigorous reflux for 18 hr (reaction can be checked by crude NMR). Mixture is then concentrated to oil on roto-vap. Oil was diluted with Ethyl ether (300 mL), washed with Sat. aqueous NaHCO₃ (2×, ~300 mL total); aqueous was washed with Ethyl ether (2×, ~300 mL total), combined organics were dried with MgSO₄, filtered and concentrated to light yellow oil; 18.86 g (69%), ¹H NMR (CDCl₃): □ 4.20-4.16 (m, 1H), 3.80 (s, 3H), 2.71 (d, 1H), 1.89-1.64 (m, 2H), 0.97 (t, 3H).

R=Methyl: can be made by starting with(S)-2-hydroxypropanic acid following the same procedures.

(R)-methyl 2-((3,5-dichloropyridin-2-yl)oxy) butanoate (3): (S)-methyl 2-hydroxybutanoate (13.18 g, 111.6 mmol), 3,5-dichloro-2-hydroxypyridine (21.96 g, 133.9 mmol, 1.2 eq), triphenyl phosphine (35.12 g, 133.9 mmol, 1.2 eq) were combined in 350 mL flask and dissolved in dry DMF (100 mL), cooled under N₂ atm. in ice bath. DIAD (26.4 mL, 133.9 mmol, 1.2 eq) diluted with DMF (25 mL) was added to the reaction mixture via addition funnel over 30 min. The resulting mixture was allowed to warm to room temperature stirring overnight. Mixture was diluted in EtOAc (200 mL), then washed with water (2×, 300 mL total), and then once with Brine (100 mL). Organics were then extracted from combined aqueous portions with EtOAc (3×, 300 mL total), the combined organics were dried with MgSO₄, filtered and adsorbed onto silica gel (~400 mL). Product was isolated by column chromatography; 0 to 15% Acetone/Hexanes linear gradient using 210 nm UV detection. Light yellow oil; 16.51 g (56%), ¹H NMR (CDCl₃): □ 7.92 (d, 1H), 7.66 (d, 1H), 5.12 (t, 1H), 3.73 (s, 3H), 2.04 (quint, 2H), 1.11 (t 3H).

(R)-2-((3,5-dichloropyridin-2-yl)oxy) butanoic acid (4): (R)-methyl 2-((3,5-dichloropyridin-2-yl)oxy) butanoate (15.01 g, 56.83 mmol) was dissolved in MeOH (200 mL) and diluted with THE (200 mL), to which was added 1M LiOH (300 mL, freshly made) via addition funnel over 2 hr, then allowed to stir at room temperature overnight. The mixture was concentrated to residue/solids on roto-vap, then diluted into water (~500 mL), washed with Ethyl ether (3×, ~400 mL total). The Ether portion was then washed with 0.7M NaOH solution (2×, ~200 mL), the combined aqueous portions were acidified to pH ~0-1 with con. HCl, then washed with DCM (4×, ~600 mL total), which was then dried with MgSO₄, filtered and solvent was removed. The residue was taken up in Ethyl ether and evaporated to solid, and dried on High-vacuum overnight. 13.81 g (97%) Off-white or light brown solid, ¹H NMR (CDCl₃): □ 11.02 (br, 1H), 7.94 (d, 1H), 7.67 (d, 1H), 5.15 (t, 1H), 2.08 (quint, 2H), 1.13 (t, 3H).

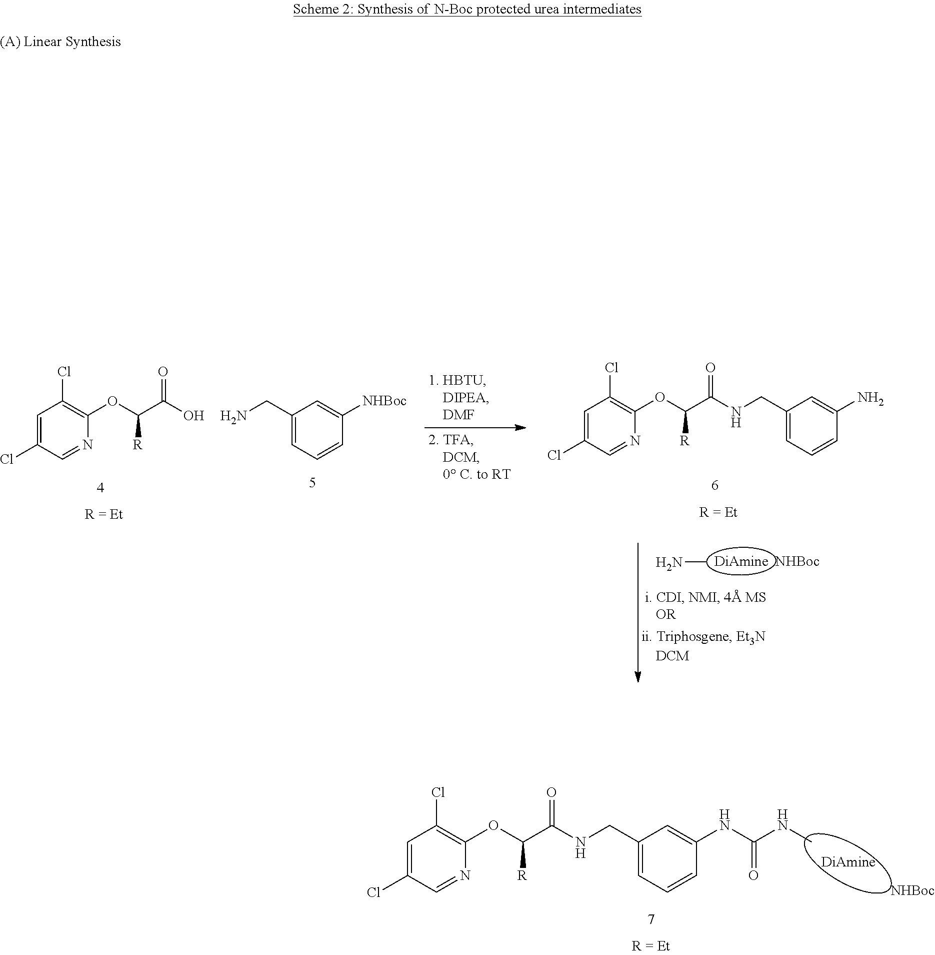

(R)-N-(3-aminobenzyl)-2-((3,5-dichloropyridin-2-yl)oxy)butanamide (6): (R)-2-((3,5-dichloropyridin-2-yl)oxy) butanoic acid 4 (1.00 g, 4.00 mmol), HBTU (1.82 g, 4.88 mmol, 1.2 eq) were dissolved in DMF (dry, 10 mL), and added diisopropylethylamine (0.91 mL, 5.20 mmol, 1.3 eq); tert-butyl 3-aminobenzylcarbamate 5 (1.07 g, 4.88 mmol, 1.2 eq) was added to the reaction mixture in 4 approx. equal portions over 5 min. The reaction was stirred at room temperature for 18 hr. Reaction mixture was diluted with water (~120 mL), precipitate was filtered and dried overnight under vacuum. 1.935 g (>99%) Off-white solid, ¹H NMR (CDCl₃): □ 7.99 (d, 1H), 7.66 (d, 1H), 7.28-7.26 (m, 1H+CHCl₃), 7.21 (d, 2H), 6.88 (br, 1H), 6.65 (br, 1H), 5.44 (t, 1H), 4.44 (d, 2H), 2.68 (m, 2H), 1.51 (s, 9H), 1.02 (t, 3H).

(R)-tert-butyl (3-((2-((3,5-dichloropyridin-2-yl)oxy)butanamido)methyl)phenyl)carbamate (1.817 g, 4.00 mmol) was dissolved in DCM (70 mL), cooled to 0° C. in ice bath and trifluoroacetic acid (20 mL) was added; and the reaction was complete by LC/MS after 3 hr. The mixture was concentrated to solid residue on rotary evaporator and dried under high vacuum overnight. The residue was then taken up in DCM (~100 mL) and washed with sat. Aq. NaHCO₃ (2×50 mL); organics were back extracted from Aq portion with 2 washes of DCM (~50 mL each), combined organics were dried with MgSO₄, filtered and concentrated to solid on rotary evaporator, then dried overnight under high vacuum. 1.103 g (78%) Light brown solid, ¹H NMR (CDCl₃): □ 18.00 (d, 1H), 7.67 (d, 1H), 7.08 (t, 1H), 6.60-6.53 (m, 4H), 5.46 (t, 1H), 4.39 (d, 2H), 2.91 (br, 2H), 2.08 (m, 2H), 1.02 (t, 3H).

### General Procedure for Making Urea:

Method A: (CDI): (R)-N-(3-aminobenzyl)-2-((3,5-dichloropyridin-2-yl)oxy)butanamide (as salt or free base) dissolved in N-methyl Imidazole (~0.15 mM), added 1 pellet/0.5 mmol of 4 Å mole sieve and was cooled to 0° C. in ice bath; then 1,1′-Carbonyldiimidazole (1.2 eq) was added and stirred in ice bath for 1 hr, after which the di-Amine (1.5 eq) was added and the mixture was allowed to warm to room temperature and diluted in water (>=10× volume of NMI); the product precipitates and was filtered and dried under vacuum overnight. {example Compound NMR}

Method B: (triphosgene): Into a solution of (R)-N-(3-aminobenzyl)-2-((3,5-dichloropyridin-2-yl)oxy)butanamide (6) (0.200 g, 0.568 mmol), Triethylamine (0.94 mL, 6.77 mmol) and DCM (11 mL) was added a solution of Triphosgene (0.167 g, 0.565 mmol) in DCM (0.3 mL) dropwise at 0° C. and the resulting mixture was allowed to stir at rt for 30 min. A solution of tert-butyl 3-(1-aminocyclopropyl) azetidine-1-carboxylate (0.143, 0.678 mmol), Triethylamine (0.55 mL), in DCM (3.6 mL) was added to the reaction mixture dropwise and the resulting reaction mixture was stirred at rt for 2 h. After completion in 2 h by LC/MS, the mixture was quenched with MeOH (5 mL) and concentrated under reduced pressure. The crude compound was purified by Combiflash by using 20 g silica gel column eluting with 0-10% MeOH/DCM gradient to give 269 mg (80%) product as a pale-yellow solid. ¹H NMR (CDCl₃): □ 07.98 (s, 1H), 7.66 (s, 1H), 7.38-7.12 (m, 4H), 6.85 (d, 1H), 6.75 (m, 1H), 5.88 (bs, 1H), 5.42-5.39 (t, 1H), 4.42 (bs, 2H), 3.97-3.91 (t, 2H), 3.68 (bs, 2H), 2.85-2.78 (m, 1H), 2.06 (m, 2H), 1.42 (s, 9H), 1.01 (t, 3H), 0.95-0.70 (m, 4H).

### (B) Convergent Synthesis

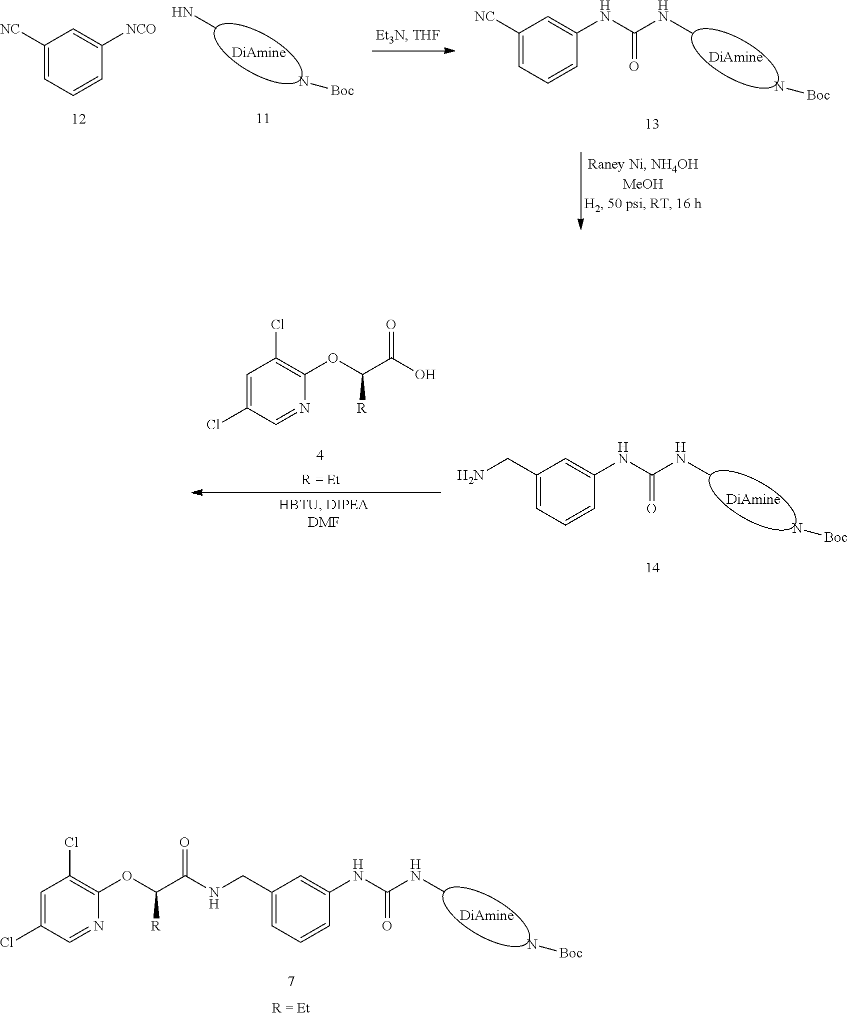

General synthesis of urea 13:3-Cyanophenylisocyanate (1.0 eq) was dissolved in THF (0.5M) and trimethylamine (1.1 eq) was added followed by mono-protected diamine (11, 1.0 eq); the reaction mixture was allowed to stir at room temperature for 18 hr. Material was then adsorbed onto Celite, and purified by silica gel column (0-100% EtOAc/Hexanes); white foam/solid. Ex.: tert-butyl 6-(3-(3-cyanophenyl)ureido)-2-azaspiro[3.3]heptane-2-carboxylate; ¹H NMR (CDCl₃): □ 7.87 (s, 1H), 7.67 (s, 1H), 7.60 (d, 1H), 7.36-7.22 (m, 2H+CHCl₃), 5.74 (br, 1H), 4.16 (m, 1H+EtOAc), 3.94 (s, 2H), 3.84 (s, 2H), 2.59 (m, 2H), 2.00 (m, 2H+EtOAc), 1.45 (s, 9H).

Nitrile Reduction (14): Nitrile-urea (13, 1.0 eq, 20-25 mmol) was dissolved in MeOH (~50 mL) in a Parr shaker vessel, to this mixture was added Raney Ni slurry (2800mesh, 5 mL) and concentrated ammonium hydroxide (5 mL). The vessel was put into Parr shaker and charged to 50 psi of H₂ gas, and shook at room temperature maintaining the 40-50 psi pressure of H₂ gas for 16 hr. The reaction mixture was carefully filtered through a Celite filter cake, and the filtrate was concentrated to yellow tinted residue/foamy solid. Ex.: tert-butyl 6-(3-(3-(aminomethyl)phenyl)ureido)-2-azaspiro[3.3]heptane-2-carboxylate; ¹H NMR (CDCl₃): □ 77.51 (br, 1H), 7.28 (m, 1H+CHCl₃), 7.19 (m, 2H), 6.90 (d, 1H), 5.72 (br, 1H), 4.10 (q, 1H), 3.86 (m, 2H), 3.76-3.62 (m, 4H), 2.51 (br t, 2H), 2.26 (br, 2H+H₂O), 1.92 (br t, 2H), 1.43 (s, 9H).

Procedure for Amide coupling for 4+14=urea (7): Into a mixture of (R)-2-((3,5-dichloropyridin-2-yl)oxy) butanoic acid 4 (0.200 g, 0.803 mmol), HBTU (0.364 g, 0.964 mmol, 1.2 eq) dissolved in DMF (dry, 2 mL), was added diisopropylethylamine (0.44 mL, 2.55 mmol, 2.5 eq) at 0° C.; after 5 min a solution of tert-butyl 6-(3-(3-(aminomethyl)phenyl)ureido)-2-azaspiro[3.3]heptane-2-carboxylate 14 (0.304 g, 0.843 mmol, 1.05 eq) in DMF (2 mL) was added; the mixture was allowed to warm to room temperature overnight. LC/MS showed reaction was complete; as well as significant guanidine side-product had formed. The reaction mixture was diluted dropwise into water (100 mL), solid precipitate was collected by vacuum filtration. Product was purified by silica chromatography (20 g prepacked column, 0-5% MeOH in DCM linear gradient; 0.312 g (66%) White solid; ¹H NMR (MeOD) 7.41 (s, 1H), 7.27 (s, 1H), 7.19-7.12 (m, 3H), 6.89-6.81 (m, 2H), 4.59-4.52 (m, 1H), 4.34 (d, 2H), 4.11-4.05 (t, 1H), 3.96 (s, 2H), 3.84 (s, 2H), 2.57 (t, 2H), 2.09 (t, 2H), 2.01-1.94 (m, 2H), 1.40 (s, 9H), 1.07 (t, 3H)

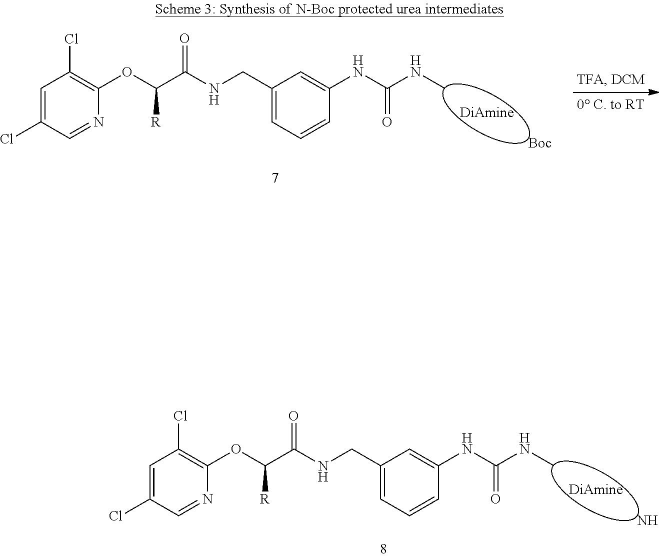

Removal of Boc-protection of di-Amine moiety (8): Dissolved the N-Boc urea (7, 1.0 eq) in DCM (0.1-0.25 mM) cooled to 0° C. in ice bath and trifluoroacetic acid (equal volume to DCM) was added; and the reaction was complete by LC/MS after 3 hr; allowed to warm to room temperature. The mixture was concentrated to solid residue on rotary evaporator and dried under high vacuum overnight. The residue was then taken up in DCM (~100 mL) and washed with sat. Aq. NaHCO₃ (2×50 mL); organics were back extracted from Aq portion with 2 washes of DCM (~50 mL each), combined organics were dried with MgSO₄, filtered and concentrated to solid on rotary evaporator. Evaporated from MeOH (2×), DCM (2×), then Et₂O (2×) and dried overnight under high vacuum. Ex.: (R)-N-(3-((2-((3,5-dichloropyridin-2-yl)oxy)butanamido)methyl)phenyl)piperazine-1-carboxamide·TFA salt ¹H NMR (MeOD-d4) δ 7.99 (s, 1H), 7.91 (s, 1H), 7.31 (s, 1H), 7.24 (m, 2H), 6.97 (m, 1H), 5.11 (1H), 4.38 (s, 2H), 3.80 (m, 4H), 3.60-3.20 (m, 4H), 2.10-1.95 (m, 2H), 1.78 (t, 3H).

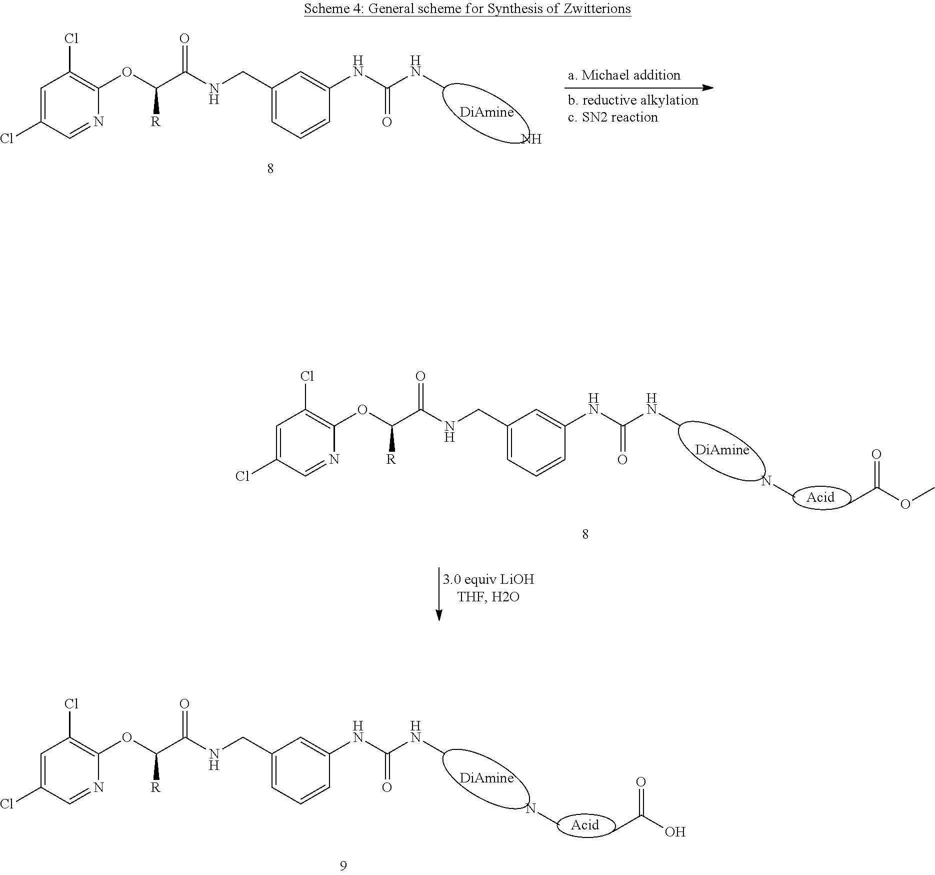

### Step 1: Addition of the Carboxylic Acid Moiety (9):

(A) (Micheal Addition): Into a solution of (R)-N-(3-(3-(1-(azetidin-3-yl)cyclopropyl)ureido)benzyl)-2-((3,5-dichloropyridin-2-yl)oxy)butanamide·TFA salt (0.05 g, 0.082 mmol), DIPEA (0.09 mL, 6.3 mmol) in MeOH (1.5 mL) was added methyl acrylate (0.015 mL, 2.12 mmol) and the reaction mixture was stirred at rt for 2 h. After 2h, reaction was completed by LC/MS. Reaction was concentrated and the crude compound was purified by combi-flash using 0-10% MeOH/DCM to give 27 mg (57%) as a colorless solid. ¹H NMR (300 MHz, MeOD) 7.96 (s, 1H), 7.89 (m, 1H), 7.32-7.17 (m, 3H), 6.88 (d, 1H), 5.08 (t, 1H), 4.64 (s, 1H), 4.33 (s, 2H), 4.03 (m, 2H), 3.89 (m, 2H), 3.76-3.69 (m, 3H), 2.91 (m, 1H), 2.63 (t, 2H), 2.02-1.98 (m, 2H), 1.08 (t, 3H), 0.84 (d, 4H).

(B) (Reductive Amination): Deprotected amine (8, 1.0 eq) was dissolved in MeOH: AcOH (10:1, 0.5 mM) mixture, to which Carbonyl (Aldehyde or Ketone, 2.0 eq) was added and mixture was stirred at room temperature for 1 hr; then Borane-2-methylpyridine complex (2.0 eq) was added and reaction mixture was allowed to stir at room temperature for 2-72 hr. The reaction was concentrated under reduced pressure to a residue, which was subsequently taken up in DCM (~10 reaction volumes) and washed with Sat Aq NaHCO₃(~20 reaction volumes). Organics were then extracted from the aqueous portion with 2 washes of DCM (10 reaction volumes each); the combined organics were dried with MgSO₄, filtered and solvent was removed. Product was purified on silica gel column (0-100% MeOH/DCM linear gradient eluent) to give white solid product. Ex: ethyl (R)-2-(4-((3-((2-((3,5-dichloropyridin-2-yl)oxy)butanamido)methyl)phenyl)carbamoyl)piperazin-1-yl)acetate; ¹H NMR (MeOD-d4) δ 8.00 (s, 1H), 7.91 (s, 1H), 7.30-7.15 (m, 3H), 6.94 (d, 1H), 5.11 (t, 1H), 4.37 (s, 2H), 4.25-4.18 (q, 2H), 3.60 (m, 4H), 3.37 (s, 2H), 2.66 (m, 4H), 2.03 (m, 2H), 1.30 (t, 3H), 1.11 (t, 3H)

(C) (S_{N}2): Amine (1.0 eq) is dissolved in DCM (0.3 mM), DiPEA (3.5 eq) was added followed by Alkyl-halide (1.05 eq); the mixture was stirred at 22° C., after 6 h the reaction was found to be complete by LC/MS. Product was purified by prep-HPLC. Ex. (R)-methyl 4-(6-(3-((2-((3,5-dichloropyridin-2-yl)oxy)butanamido)methyl)benzamido)-2-azaspiro[3.3]heptan-2-yl) butanoate 0.023 g (38%) white solid; ¹H NMR (CDCl₃) d 12.35 (br, 1H), 7.96 (d, 1H), 7.66-7.62 (m, 3H), 7.30-7.26 (m, 2H+CHCl₃), 7.17 (d, 1H), 6.94 (br, 1H), 5.39 (t, 1H), 4.46-4.36 (m, 5H), 3.82-3.68 (m, 5H), 3.11 (br, 2H), 2.76 (m, 1H), 2.65 (m, 1H), 2.47-2.30 (m, 4H), 2.08-2.01 (m, 2H), 1.88-1.80 (m, 2H), 1.01 (t, 3H).

### Step 2: Hydrolysis of Ester (10):

Carboxylic acid ester (9, 1.0 eq) was dissolved in THF (0.1M) to which 1M LiOH Aq solution (3.0 eq) was added and stirred at room temperature for 4-18 hr. Material was adsorbed onto Celite and purified on silica gel column (0-100% MeOH/DCM linear gradient); white solid product. Ex.: lithium (R)-2-(6-(3-(3-((2-((3,5-dichloropyridin-2-yl)oxy)butanamido)methyl)phenyl)ureido)-2-azaspiro[3.3]heptan-2-yl)acetate; ¹H NMR (DMSO): □ 9.31 (s, 1H), 8.49 (t, 1H), 8.15 (m, 2H), 7.48 (d, 1H), 7.32 (s, 1H), 7.26 (d, 1H), 7.11 (t, 1H), 6.72 (d, 1H), 5.08 (dd, 1H), 4.20 (m, 2H), 4.01 (dt, 1H), 3.89 (s, 2H), 3.96 (s, 2H), 2.56 (m, 2H), 2.10 (m, 2H), 1.91 (m, 2H), 0.99 (t, 3H).

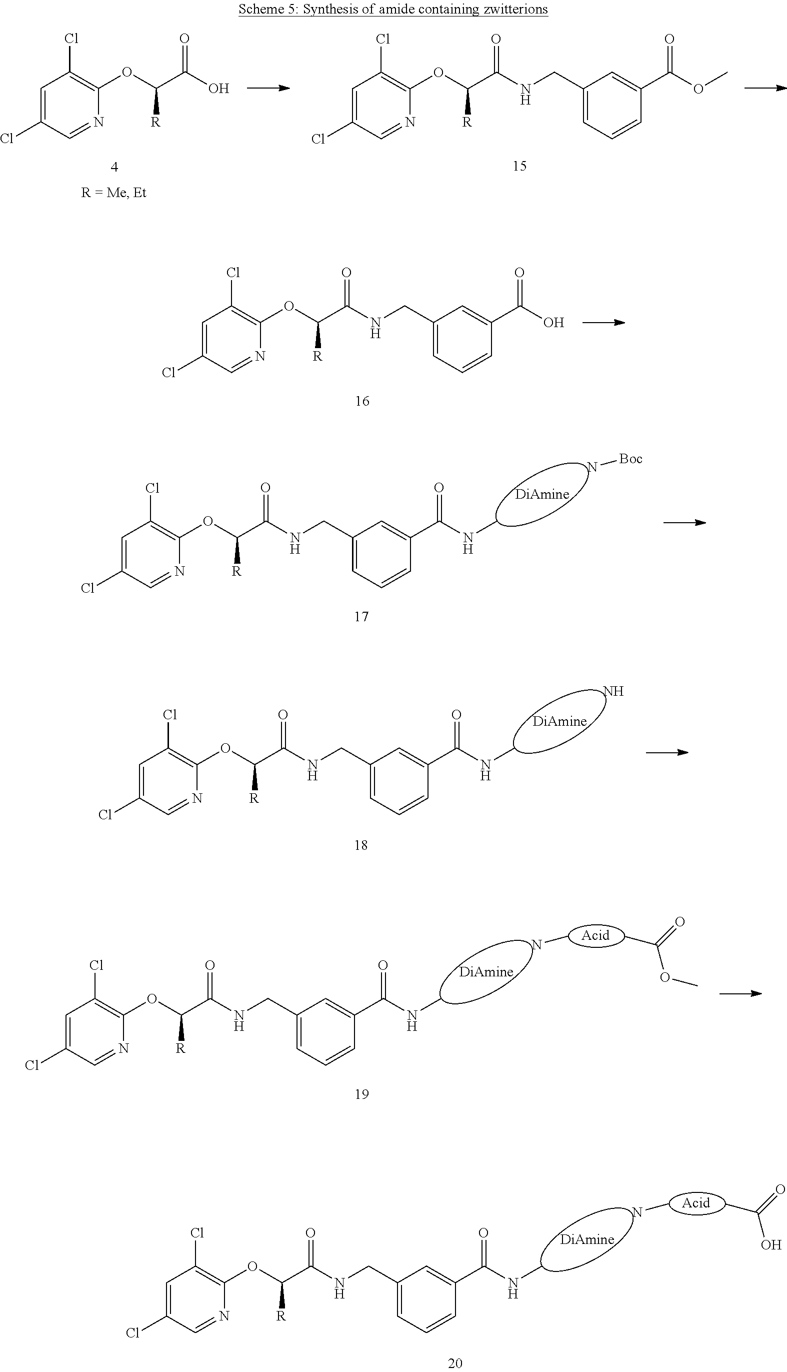

(R)-methyl 3-((2-((3,5-dichloropyridin-2-yl)oxy)butanamido)methyl)benzoate (15): (R)-2-((3,5-dichloropyridin-2-yl)oxy) butanoic acid (15.00 g, 59.98 mmol), HBTU (27.30 g, 71.98 mmol, 1.2 eq) were dissolved in DMF (dry, 300 mL), cooled in ice bath; a solution of methyl 3-(aminomethyl)benzoate hydrochloride (14.51 g, 71.98 mmol, 1.2 eq), diisopropylethyl amine (26.1 mL, 150 mmol, 2.5 eq) was added to the solution of HBTU and (R)-2-((3,5-dichloropyridin-2-yl)oxy) butanoic acid via addition funnel over 2 hr, then allowed to stir at room temperature for 16-18 hr. Reaction mixture was diluted with water (~4 L), precipitate was filtered and dried overnight under vacuum. 26.36 g (>99%) Light brown solid, ¹H NMR (CDCl₃): □ 7.95-7.90 (m, 3H), 7.67 (d, 1H), 7.45-7.36 (m, 2H), 6.70 (br, 1H), 5.45 (t, 1H), 4.53 (d, 2H), 3.91 (s, 3H), 2.14-2.04 (m, 2H), 1.03 (t, 3H).

(R)-3-((2-((3,5-dichloropyridin-2-yl)oxy)butanamido)methyl)benzoic acid (16): (R)-methyl 3-((2-((3,5-dichloropyridin-2-yl)oxy)butanamido)methyl)benzoate (26.36 g, 66.36 mmol) was taken up in THF (200 mL) to which 2M Aq NaOH solution (200 mL) and additional THF (100 mL) was added; the mixture was stirred at room temperature for 16-18 hr. The mixture was concentrated on roto-vap, and the residue was dissolved in water (~200 mL) and was washed with DCM three times (~500 mL total volume); the aqueous portion was acidified to pH 1 with con. HCl. The precipitate was filtered and dried in vacuum oven overnight at 65° C. in the presence of P₂O₅; 23.00 g (>99%) Off-white solid; NMR (DMSO): □ 12.93 (br, 1H), 8.62 (t, 1H), 8.15 (m, 2H), 7.85-7.80 (m, 2H), 7.45-7.40 (m, 2H), 5.08 (t, 1H), 4.34 (qd, 2H), 1.90 (m, 2H), 1.00 (t, 3H).

Amide formation (17): (R)-3-((2-((3,5-dichloropyridin-2-yl)oxy)butanamido)methyl)benzoic acid (1.0 eq) and HBTU (1.2 eq) was dissolved in DMF (0.25 mM), then diisopropylethyl amine (1.3 eq) and mono-Boc diamine (1.1 eq) were added; the mixture was allowed to stir at room temperature for 16-18 hr. The reaction was diluted into water (>10 reaction volumes) in a dropwise manner; the precipitated product was filtered and dried in the vacuum oven overnight at 70° C. in the presence of P₂O₅; white/off-white solid. Ex: (R)-tert-butyl 6-(3-((2-((3,5-dichloropyridin-2-yl)oxy)butanamido)methyl)benzamido)-2-azaspiro[3.3]heptane-2-carboxylate; ¹H NMR (DMSO): □ 8.63-8.57 (m, 2H), 8.17 (m, 2H), 7.72-7.66 (m, 2H), 7.39-7.36 (m, 2H), 5.06 (t, 1H), 4.36-4.25 (m, 3H), 3.91 (s, 2H), 3.79 (s, 2H), 2.23 (t, 2H), 1.89 (m, 2H), 1.37 (s, 9H), 1.00 (t, 3H).

Steps to make Compound 18, 19, and 20 are the same as for Compounds 8, 9, and 10.

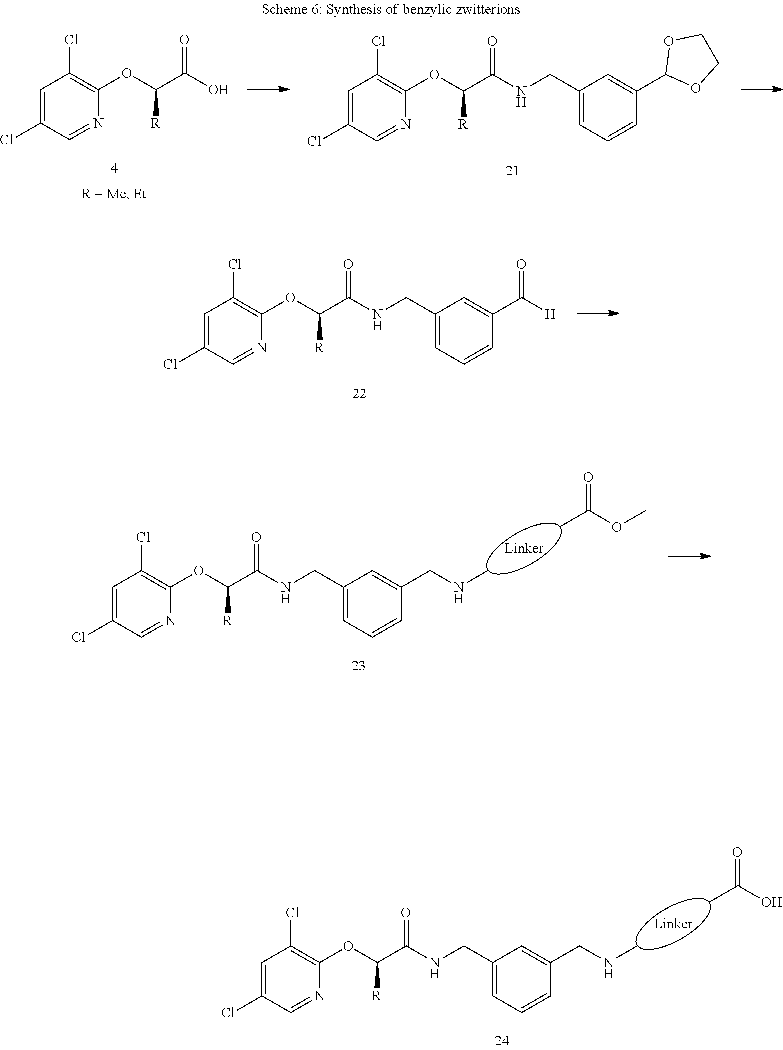

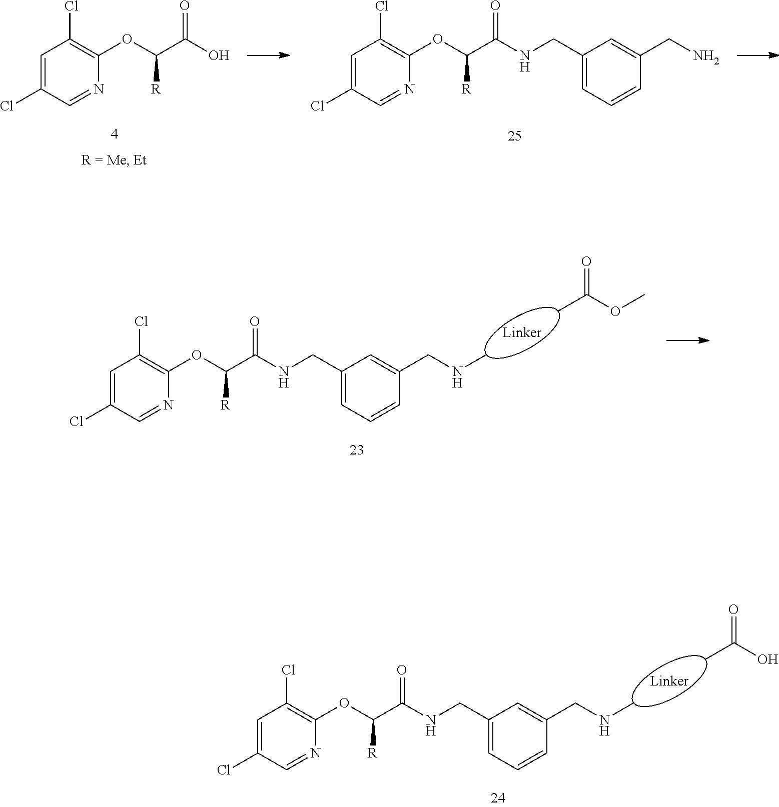

(R)-2-((3,5-dichloropyridin-2-yl)oxy)-N-(3-formylbenzyl)butanamide (22): (R)-2-((3,5-dichloropyridin-2-yl)oxy) butanoic acid (1.0 eq), HBTU (1.2 eq) were dissolved in DMF (0.1 mM) and added diisopropylethyl amine (1.3 eq) and stirred at room temperature for 30 min; then (3-(1,3-dioxolan-2-yl)phenyl)methanamine (1.1 eq) dissolve in DMF (3 mM) was added dropwise at a rate of 2 mL/min; the mixture was allowed to remain stirring at room temperature for 16-18 hr. Product was precipitated by the dilution of the reaction mixture into water (>10 reaction volumes) in a dropwise manner; the precipitate was filtered and dried under vacuum. The resulting (R)-N-(3-(1,3-dioxolan-2-yl)benzyl)-2-((3,5-dichloropyridin-2-yl)oxy)butanamide (21) solid is suspended in DCM (0.5 mM), to which Trifluoroacetic acid (20 eq) was added over 10 min period, and the mixture was stirred at room temperature for 2 hr. The reaction was quenched by shaking in separatory funnel with water (2 reaction volumes); the aqueous portion was subsequently washed with EtOAc (3 times), and the combined organics were washed with brine, then concentrated to solid. Passing material through silica gel column using a 0-50% EtOAc in Hexanes linear gradient gave an oily residue that was taken up in DCM and washed with Sat. Aq. NaHCO₃, and extracted material from Aqueous portion with EtOAc (2×), the combined organics were dried with MgSO₄, filtered and concentrated to solid product; White solid; ¹H NMR (CDCl₃): □ 9.98 (s, 1H), 8.01 (d, 1H), 7.79-7.73 (m, 2H), 7.68 (d, 1H), 7.53-7.45 (m, 2H), 6.77 (br, 1H), 5.46 (t, 1H), 4.66-4.50 (m, 2H), 2.14-2.04 (m, 2H), 1.03 (t, 3H).

(R)-N-(3-(aminomethyl)benzyl)-2-((3,5-dichloropyridin-2-yl)oxy)butanamide (25): (R)-2-((3,5-dichloropyridin-2-yl)oxy) butanoic acid (0.500 g, 1.999 mmol), HBTU (0.910 g, 2.399 mmol, 1.2 eq) were dissolved in DMF (dry, 4 mL), and added diisopropylethyl amine (0.45 mL, 2.599 mmol, 1.3 eq). tert-butyl 3-(aminomethyl)benzylcarbamate (0.567 g, 2.399 mmol, 1.2 eq) was dissolved in DMF (dry, 1 mL) and added to the reaction mixture in a dropwise fashion via syringe. The reaction was stirred at room temperature for 18 hr. Reaction mixture was diluted with water (~70 mL), precipitate was filtered and dried under vacuum. Product was isolated by column chromatography; 0 to 75% EtOAc/Hexanes linear gradient. 0.807 g (86%) White solid, ¹H NMR (CDCl₃): 8.00 (d, 1H), 7.68 (d, 1H), 7.30-7.11 (m, 4H+CHCl₃), 6.67 (br t, 1H), 5.47 (t, 1H), 4.83 (br, 1H), 4.47 (d, 2H), 4.28 (d, 2H), 2.11-2.06 (m, 2H), 1.46 (s, 9H), 1.02 (t, 3H). (R)-tert-butyl 3-((2-((3,5-dichloropyridin-2-yl)oxy)butanamido)methyl)benzylcarbamate (5.619 g, 12.00 mmol) was dissolved in DCM (50 mL), cooled in ice bath and 4.0M HCl in dioxane (200 mL) was added via addition funnel. The reaction was complete after 4 hr, and the mixture was diluted with Ethyl ether (500 mL), the solid precipitate was filtered and dried under vacuum. Solid was then taken up into MeOH (100 mL) and evaporated, twice. The resulting solid was triturated with Ethyl ether, filtered and dried under High vacuum. >5 g recovered (>100% yield, material still retained some dioxane, which can be removed by lyophylization), Light brown solid, ¹H NMR (DMSO): 8.72 (t, 1H), 8.59 (br, 3H), 8.21 (d, 1H), 8.15 (d, 1H), 7.42-7.22 (m, 4H), 5.11 (t, 1H), 4.28 (d, 2H), 3.95 (d, 2H), 1.95-1.90 (m, 2H), 1.00 (t, 3H). HCl salt and excess dioxane were removed by portioning between DCM and Sat. Aq. NaCO₃ solution, aqueous portion was then washed 2 more times with DCM, the combined organics were dried with MgSO₄, filtered and solvent was removed and dried under vacuum.

### Reductive Amination (23):

(A) Benzaldehyde: (R)-2-((3,5-dichloropyridin-2-yl)oxy)-N-(3-formylbenzyl)butanamide (1.0 eq) and Amino-acid (1.1 eq) were dissolved in MeOH (0.1-0.2 mM), a catalytic amount of acetic acid (~1% reaction volume) and NaHB(OAc)₃ (1.2 eq) were added and the mixture was stirred at room temperature and monitored by LC/MS. When reaction is shown to be complete, the product was purified by prep-HPLC, and freeze dried.

(B) Benzylamine: (R)-N-(3-(aminomethyl)benzyl)-2-((3,5-dichloropyridin-2-yl)oxy)butanamide (1.0 eq) and aldehyde or ketone (1.1 eq) were dissolved in DCM:MeOH (1:1 mixture, 0.1-0.2 mM), a catalytic amount of acetic acid (~1% reaction volume) and NaHB(OAc) 3 (1.2 eq) were added and the mixture was stirred at room temperature and monitored by LC/MS. When reaction is shown to be complete, the product was purified by prep-HPLC, and freeze dried.

### Ester Removal (24): Same Preparation as for Compound (10).

TABLE 3
Analytical data for analogs

| | | m/z found |
|---|---|---|
| Compd No. | | by LC/MS |
| MBX | 1H NMR spectrum (solvent) | (M + x) |
| 7531A | (DMSO) 8.74 (br s, 1H), 8.54 (s, 1H), 8.16 (d, 2H), 7.31-7.20 | 568.0 |
| | (m, 2H), 7.15-7.10 (t, 1H), 6.73 (d, 1H), 6.68-6.53 | |
| | (m, 1H), 5.08-5.04 (t, 1H), 4.31-4.10 (m, 2H), 3.84-3.75 | |
| | (t, 2H), 3.70-3.50 (m, 2H + MeOH), 3.08-2.92 (m, 2H), | |
| | 2.90-2.76 (m, 1H), 2.75-2.62 (m, 2H), 2.33-2.23 (m, 2H), | |
| | 2.17-2.03 (m, 1H), 1.98-1.83 (m, 2H), 1.01-0.96 (t, 3H) | |
| 7497A | (DMSO) 9.99 (s, 1H), 8.56-8.47 (m, 1H), 8.39-8.26 (m, | 498.0 |
| | 1H), 8.17 (s, 2H), 7.39 (s, 1H), 7.35-7.27 (m, 1H), 7.13-7.08 | |
| | (t, 1H), 6.70 (d, 1H), 5.09-5.05 (t, 1H), 4.29-4.11 (m, | |
| | 2H), 3.60-3.14 (m, 5H), 2.80 (s, 2H), 2.00-1.84 (m, 2H), | |
| | 1.01-0.96 (t, 3H) | |
| 7496A | DMSO) 9.17 (s, 1H), 8.61-8.51 (m, 1H), 8.23-8.13 (m, | 512.1 |
| | 2H), 7.37-7.23 (m 2H), 7.15-7.09 (t, 1H), 6.99 (br s, 1H), | |
| | 6.72 (d, 1H), 5.09-5.05 (t, 1H), 4.29-4.12 (m, 2H), 3.77-3.08 | |
| | (m, 3H), 2.82 (s, 2H), 2.74 (s, 2H), 2.26-2.22 (t, 2H), | |
| | 1.98-1.84 (m, 2H), 1.01-0.96 (t, 3H) | |
| 7495A | DMSO) 8.68 (s, 1H), 8.59-8.48 (m, 1H), 8.16 (d, 2H), | 564.1 |
| | 7.32 (s, 1H), 7.25-7.17 (m, 1H), 7.14-7.08 (t, 1H), 7.02 (s, | |
| | 1H), 6.72 (d, 1H), 5.08-5.04 (t, 1H), 4.29-4.11 (m, 2H), | |
| | 3.36-3.31 (t, 2H), 2.92-2.87 (t, 2H), 2.68-2.54 (m, 3H), | |
| | 2.10-2.05 (t, 2H), 1.98-1.83 (m, 2H), 1.01-0.96 (t, 3H), | |
| | 0.66 (s, 4H) | |
| 7478A | DMSO) 9.00 (s, 1H), 8.15 (br s, 1H), 7.52 (d, 1H), 7.31-7.20 | 597.2 |
| | (m, 2H), 7.15-7.05 (m, 2H), 6.81 (d, 1H), 6.65 (d, | |
| | 1H), 4.65-4.54 (m, 1H), 4.04-3.81 (m, 5H), 3.69-3.10 (m, | |
| | 4H), 2.70-2.45 (m, 4H), 2.14-2.04 (m, 2H), 1.91-1.78 (m, | |
| | 2H), 0.95-0.90 (t, 3H) | |
| 7477A | (DMSO) 8.63 (s, 1H), 8.15 (br s, 1H), 7.53 (d, 1H), 7.25 | 611.1 |
| | (s, 1H), 7.18 (d, 1H), 7.13-7.01 (m, 1H), 6.82 (d, 1H), | |
| | 6.78-6.64 (m, 2H), 4.63-4.53 (m, 1H), 4.03-3.90 (m, 1H), | |
| | 3.71-3.26 (m, 4H), 3.21 (s, 2H), 3.12 (s, 2H), 2.68-2.47 | |
| | (m, 2H), 2.43-2.30 (m, 2H), 2.40-2.03 (m, 2H), 2.0-1.90 | |
| | (m, 2H), 1.88-1.79 (m, 2H), 0.95-0.90 (t, 3H) | |
| 7476A | (MeOD) 7.35 (d, 1H), 7.28 (br s, 1H), 7.21-7.10 (m, 2H), | 583.0 |
| | 6.89-6.79 (m, 2H), 4.61-4.57 (t, 1H), 4.40-4.27 (m, 2H), | |
| | 4.18-4.07 (m, 3H), 4.01 (s, 2H), 3.64 (s, 2H), 2.70-2.65 (t, | |
| | 2H), 2.23-2.16 (t, 2H), 2.08-1.94 (m, 2H), 1.10-1.05 (t, | |
| | 3H) | |
| 7464A | (DMSO) 9.08 (s, 1H), 8.16 (s, 1H), 8.11 (s, 1H), 8.06-7.97 | 564.2 |
| | (m, 1H), 7.30-7.19 (m, 3H), 7.08-7.03 (t, 1H), 6.65 (d, | |
| | 1H), 5.01-4.97 (t, 1H), 4.04-3.91 (m, 1H), 3.89-3.75 (m, | |
| | 4H), 3.66-3.16 (m, 6H), 2.66-2.41 (m, 2H), 2.11-2.0 (m, | |
| | 2H), 1.90-1.75 (m, 2H), 0.94-0.89 (t, 3H) | |
| 7463A | (DMSO) 8.71 (s, 1H), 8.11 (s, 1H), 8.06-7.96 (m, 1H), | 578.1 |
| | 7.27-7.14 (m, 2H), 7.09-7.3 (t, 1H), 6.90-6.79 (m, 1H), | |
| | 6.65 (d, 1H), 5.01-4.97 (t, 1H), 4.02-3.87 (m, 1H), | |
| | 3.28-3.08 (m, 6H), 2.65-2.46 (m, 4H), 2.42-2.29 (m, 2H), | |
| | 2.13-2.03 (m, 2H), 2.0-1.89 (m, 2H), 1.86-1.76 (m, 2H), | |
| | 0.94-0.89 (t, 3H) | |
| 7439A | (DMSO) 8.59-8.45 (m, 2H), 8.14 (d, 2H), 7.29-7.18 (m, | 550.1 |
| | 2H), 7.13-7.08 (t, 1H), 6.72 (d, 1H), 6.66-6.56 (m, 1H), | |
| | 5.28-5.18 (m, 1H), 4.26-4.11 (m, 2H), 4.02-3.89 (m, 1H), | |
| | 3.21 (s, 2H), 3.12 (s, 2H), 2.59-2.54 (t, 2H), 2.42-2.31 (m, | |
| | 2H), 2.11-2.07 (t, 2H), 1.96-1.90 (t, 2H), 1.50 (d, 3H) | |
| 7438A | (DMSO) 9.88 (s, 1H), 8.58-8.46 (m, 1H), 8.28-8.13 (m, | 524.2 |
| | 3H), 7.39 (s, 1H), 7.30 (d, 1H), 7.13-7.07 (t, 1H), 6.70 (d, | |
| | 1H), 5.09-5.05 (t, 1H), 4.27-4.11 (m, 2H), 3.94-3.80 (m, | |
| | 2H), 3.77-3.65 (m, 2H), 3.62-3.09 (m, 4H), 2.77-2.64 (m, | |
| | 1H), 2.00-1.84 (m, 2H), 1.01-0.96 (t, 3H) | |
| 7437A | (MeOD) 7.96 (s, 1H), 7.88 (s, 1H), 7.32-7.13 (m, 3H), | 538.2 |
| | 6.87 (d, 1H), 5.11-5.07 (t, 1H), 4.33 (s, 2H), 4.05-3.99 (t, | |
| | 2H), 3.78-3.73 (t, 2H), 3.39 (d, 2H), 3.22-3.17 (t, 2H), | |
| | 3.10-2.91 (m, 1H), 2.37-2.33 (t, 2H), 2.02-1.89 (m, 2H), | |
| | 1.10-1.05 (t, 3H) | |
| 7436A | (DMSO) 9.28 (s, 1H), 8.66-8.62 (t, 1H), 7.58 (s, 1H), | 549.1 |
| | 7.48-7.30 (m, 3H), 7.22 (d, 1H), 7.12-7.07 (t, 1H), 7.96 (d, | |
| | 1H), 6.68 (d, 1H), 4.62-4.58 (t, 1H), 4.21 (d, 2H), | |
| | 4.06-3.94 (m, 1H), 3.90 (s, 2H), 3.83 (s, 2H), 3.39 (s, 2H), | |
| | 2.61-2.46 (m, 2H), 2.15-2.03 (m, 2H), 1.95-1.82 (m, 2H), | |
| | 1.00-0.96 (t, 3H) | |
| 7435A | (DMSO) 8.87 (s, 1H), 8.64 (br s, 1H), 7.58 (s, 1H), | 563.1 |
| | 7.42-7.31 (m, 2H), 7.26-7.16 (m, 1H), 7.12-7.07 (t, 1H), | |
| | 7.01-6.90 (m, 2H), 6.69 (d, 1H), 4.62-4.58 (t, 1H), 4.29-4.18 | |
| | (m, 2H), 4.05-3.90 (m, 1H), 3.20 (s, 2H), 3.13 (s, 2H), | |
| | 2.63-2.53 (m, 2H), 2.44-2.32 (m, 2H), 2.09-2.04 (t, 2H), | |
| | 2.00-1.82 (m, 4H), 1.01-0.96 (t, 3H) | |
| 7434A | (DMSO) 9.13 (s, 1H), 8.52 (br s, 1H), 8.17 (d, 2H), 7.35-7.21 | 536.2 |
| | (m, 3H), 7.14-7.09 (t, 1H), 6.72 (d, 1H), 5.32-5.18 | |
| | (m, 1H), 4.31-4.12 (m, 2H), 4.09-3.96 (m, 1H), 3.87 (s, | |
| | 2H), 3.83 (s, 2H), 3.72-3.16 (m, 2H + H2O), 2.40-2.60 (m, | |
| | 2H), 2.17-2.00 (m, 2H), 1.53 (d, 3H) | |
| 7424A | (MeOD) 7.95 (s, 1H), 7.88 (s, 1H), 7.30 (s, 1H), 7.29-7.21 | 590.2 |
| | (m, 1H), 7.19-7.14 (t, 1H), 6.89 (d, 1H), 5.10-5.06 (t, 1H), | |
| | 4.33 (s, 2H), 4.29-4.15 (m, 8H), 3.33 (s, 2H), 2.42-2.38 | |
| | (m, 2H), 2.11-1.85 (m, 6H), 1.10-1.05 (t, 3H) | |
| 7423A | (MeOD) 7.95 (s, 1H), 7.89 (s, 1H), 7.31 (s, 1H), 7.29-7.22 | 576.2 |
| | (m, 1H), 7.21-7.13 (m, 1H), 6.89 (d, 1H), 5.10-5.06 (t, | |
| | 1H), 4.33 (s, 6H), 4.23 (s, 4H), 3.20 (s, 2H), 2.04-1.95 (m, | |
| | 2H), 1.15 (s, 2H), 1.10-1.05 (t, 3H), 0.74 (s, 2H) | |
| 7422A | (DMSO): 8.91 (br s, 1H), 8.18 (d, 1H), 8.09 (d, 1H), 7.33 | 568.1 |
| | (t, 1H), 7.12-7.07 (m, 3H), 5.80-5.78 (t, 1H), 5.13 (t, 1H), | |
| | 4.36-4.23 (m, 2H), 3.70-3.67 (m, 2H + H2O), 3.11 (s, 3H), | |
| | 3.06-3.02 (m, 2H), 2.89-2.79 (m, 3H), 2.66 (d, 1H), | |
| | 2.17-2.11 (m, 1H), 1.95-1.79 (m, 3H), 1.00 (t, 3H) | |
| 7421A | (DMSO): 9.15 (br, 1H), 8.52 (t, 1H), 8.17-8.14 (m, 2H), | 540.2 |
| | 7.31-7.26 (m, 2H), 7.13 (t, 1H), 6.74 (d, 1H), 5.07 (t, 1H), | |
| | 4.32-4.13 (m, 3H), 3.69-3.64 (m, 2H), 3.60-3.40 (1H + | |
| | H2O), 3.20 (br, 2H), 2.88-2.85 (m, 1H), 2.69 (br, 1H), | |
| | 1.91 (quint, 2H), 0.99 (t, 3H) | |
| 7420A | (DMSO): 9.08 (br, 2H), 8.57 (br, 1H), 8.17 (d, 2H), 7.45 | 574.1 |
| | (s, 2H), 7.38-7.32 (m, 2H), 7.28-7.18 (m, 2H), 6.97 (d, | |
| | 1H), 6.82 (d, 1H), 5.09 (t, 1H), 4.32-4.19 (m, 2H), 3.82 (s, | |
| | 2H), 2.83-2.81 (m, 2H), 2.33 (t, 2H), 1.95 (m, 2H), 1.01 (t, | |
| | 3H) | |
| 7419A | (DMSO): 10.25-10.21 (m, 2H), 8.56 (m, 1H), 8.17 (s, 2H), | 560.0 |
| | 7.80 (d, 1H), 7.39 (s, 2H), 7.38 (d, 1H), 7.28 (t, 1H), 7.18 | |
| | (t, 1H), 7.01 (d, 1H), 6.80 (d, 1H), 5.09 (t, 1H), 4.25-4.24 | |
| | (m, 2H), 4.03 (s, 2H), 3.90-3.10 (2H + H2O), 1.92 (q, | |
| | 2H), 1.01 (t, 3H) | |
| 7394A | (DMSO): 8.59-8.49 (m, 2H), 8.18 (d, 1H), 8.15 (d, 1H), | 564.2 |
| | 7.26-7.23 (m, 2H), 7.15-7.10 (m, 1H), 6.74 (d, 1H), 6.65-6.63 | |
| | (m, 1H), 5.07 (t, 1H), 4.26-4.13 (m, 2H), 4.02-3.94 | |
| | (m, 1H), 3.24 (s, 2H), 3.15 (s, 2H), 2.62-2.59 (m, 2H), | |
| | 2.42-2.35 (m, 2H), 2.12 (t, 2H), 1.99-1.86 (m, 4H), 1.00 (t, | |
| | 3H) | |
| 7393A | (DMSO): 9.18 (d, 1H), 8.51 (t, 1H), 8.17 (d, 2H), 7.30-7.25 (m, 3H), | 550.1 |
| | 7.12 (t, 1H), 6.72 (d, 1H), 5.07 (t, 1H), 4.20-4.12 (m, 2H), 3.99 (s, | |
| | 2H), 3.94 (s, 2H), 3.51 (s, 2H), 2.61-2.54 (m, 1H + DMSO), 2.13-2.10 | |
| | (m, 2H), 1.93-1.89 (m, 2H), 0.99 (t, 3H) | |
| 7388A | (300 MHz, DMSO) 8.78 (s, 1H), 8.50 (s, 1H), 8.15 (d, 2H), 7.31-7.19 | 564.3 |
| | (m, 2H), 7.13-7.08 (t, 1H), 6.86 (d, 1H), 6.72 (d, 1H), 5.11-5.03 (m, | |
| | 1H), 4.31-4.11 (m, 2H), 4.06-3.91 (m, 1H), 3.25 (s, 2H), 3.16 (s, 2H), | |
| | 2.66-2.56 (m, 2H), 2.44-2.33 (m, 2H), 2.17-2.05 (m, 2H), 2.03-1.86 | |
| | (m, 4H), 1.01-0.96 (t, 3H) | |
| 7353A | (DMSO) 8.55 (br s, 2H), 8.18 (s, 1H), 8.15 (s, 1H), 7.25 | 554.0 |
| | (br s, 2H), 7.17-7.12 (t, 1H), 6.75 (d, 1H), 6.32-6.20 (m, | |
| | 1H), 5.13-5.02 (m, 1H), 4.30-4.14 (m, 2H), 3.88-3.75 (m, | |
| | 2H), 3.23-3.03 (m, 5H), 2.81 (d, 1H), 2.71 (d, 1H), | |
| | 2.38-2.24 (m, 1H), 2.13-2.00 (m, 1H), 1.98-1.84 (m, 2H), | |
| | 1.02-0.98 (t, 3H) | |
| 7352A | (MeOD) 8.75 (br s, 1H), 8.53 (br s, 1H), 8.16 (d, 2H), | 554.0 |
| | 7.27 (s, 2H), 7.16-7.11 (t, 1H), 6.74 (d, 1H), 6.40 (br s, | |
| | 1H), 5.16-5.03 (m, 1H), 4.31-4.13 (m, 2H), 3.88-3.76 (m | |
| | 2H), 3.24-3.00 (m, 5H), 2.85 (d, 1H), 2.71 (d, 1H), | |
| | 2.36-2.21 (m, 1H), 2.11-1.83 (m, 3H), 1.02-0.97 (t, 3H) | |
| 7341A | (MeOD) 8.54 (br s, 1H), 7.98 (s, 1H), 7.91 (s, 1H), | 538.2 |
| | 7.33-7.16 (m, 3H), 6.90 (d, 1H), 5.13-5.09 (t, 1H), 4.36 (s, 2H), | |
| | 4.20-4.04 (m, 1H), 3.71-3.46 (m, 3H), 3.17-2.94 (m, 2H), | |
| | 2.68 (s, 1H), 2.14-1.85 (m, 5H), 1.71-1.53 (m, 1H), | |
| | 1.13-1.08 (t, 3H) | |
| 7340A | (MeOD) 7.98 (s, 1H), 7.91 (s, 1H), 7.33-7.16 (m, 3H), | 538.2 |
| | 6.90 (d, 1H), 5.13-5.09 (t, 1H), 4.36 (s, 2H), 4.16-4.02 (m, | |
| | 1H), 3.63-3.4 (m, 3H), 3.12-2.86 (m, 2H), 2.68 (s, 1H), | |
| | 2.11-1.84 (m, 5H), 1.67-1.51 (m, 1H), 1.13-1.08 (t, 3H) | |
| 7339A | (MeOD) 8.56 (s, 1H), 7.98 (s, 1H), 7.91 (s, 1H), 7.35-7.16 | 566.2 |
| | (m, 3H), 6.90 (d, 1H), 5.14-5.10 (t, 1H), 4.49-4.32 (m, | |
| | 3H), 3.76-3.63 (m, 1H), 3.48 (s, 2H), 3.21 (s, 2H), | |
| | 2.56-2.40 (m, 1H), 2.14-1.99 (m, 3H), 1.37-1.22 (m, 7H), | |
| | 1.13-1.08 (t, 3H) | |
| 7326A | (DMSO): 8.51 (t, 1H), 8.37 (s, 1H), 8.19-8.15 (m, 2H), | 524.1 |
| | 7.38-7.34 (m, 2H), 7.16 (t, 1H), 6.79 (d, 1H), 5.08 (t, 1H), | |
| | 4.24-4.19 (m, 2H), 4.22 (t, 2H), 4.08-4.03 (m, 2H), | |
| | 3.52-3.48 (m, 2H + H2O), 2.68 (t, 2H), 2.32 (t, 2H), 1.93 | |
| | (quint, 2H), 1.00 (t, 3H) | |
| 7307A | (DMSO) 8.61 (br s, 1H), 8.52 (br s, 1H), 8.16 (d, 2H), | 552.2 |
| | 7.34-7.21 (m, 2H), 7.19-7.08 (m, 1H), 6.74 (d, 1H), 6.32 | |
| | (d, 1H), 5.15-5.05 (m, 1H), 4.28-4.13 (m, 2H), 3.76-3.64 | |
| | (m, 1H), 2.75-2.55 (m, 3H), 2.40-2.21 (m, 4H), 2.20-2.05 | |
| | (m, 1H), 1.99-1.86 (m, 2H), 1.75-1.58 (m, 2H), 1.55-1.43 | |
| | (m, 1H), 1.37-1.22 (m, 1H), 1.02-0.7 (t, 3H) | |
| 7306A | (DMSO) 8.64-8.48 (m, 2H), 8.15 (d, 2H), 7.31-7.21 (m, | 552.2 |
| | 2H), 7.15-7.10 (t, 1H), 6.73 (d, 1H), 6.27 (d, 1H), | |
| | 5.14-5.03 (m, 1H), 4.28-4.16 (m, 2H), 3.75-3.60 (m, 1H), | |
| | 2.69-2.53 (m, 3H), 2.40-2.20 (m, 4H), 2.18-1.85 (m, 3H), | |
| | 1.71-1.56 (m, 2H), 1.57-1.22 (m, 2H), 1.01-0.97 (t, 3H) | |
| 7305A | (DMSO): 8.51-8.50 (m, 1H), 8.38 (s, 1H), 8.16 (d, 2H), | 510.1 |
| | 7.39-7.34 (m, 2H), 7.15 (t, 1H), 6.78 (d, 1H), 5.08 (t, 1H), | |
| | 4.27-4.14 (m, 2H), 4.05-4.00 (m, 2H), 3.70-3.67 (m, 2H), | |
| | 3.56-3.54 (m, 2H), 3.00 (s, 2H), 1.93-1.91 (m, 2H), 1.00 | |
| | (t, 3H) | |
| 7304A | (DMSO): 8.66 (br s, 1H), 8.64-8.62 (m, 1H), 8.53-8.50 | 552.3 |
| | (m, 2H), 7.28-7.24 (m, 2H), 7.13 (t, 1H), 6.74 (d, 1H), | |
| | 6.52 (br s, 1H), 5.08 (t, 1H), 4.29-4.13 (m, 2H), 3.50 (1H + | |
| | H2O), 2.86 (br, 2H), 2.67-2.63 (m, 2H), 2.38-2.33 (m, | |
| | 2H), 2.28-2.25 (m, 2H), 1.94-1.86 (m, 4H), 1.44-1.34 (m, | |
| | 2H), 1.02 (t, 3H) | |
| 7303A | (DMSO): 8.64 (br s, 1H), 8.54-8.50 (m, 1H), 8.19-8.15 | 538.2 |
| | (m, 2H), 7.28-7.24 (m, 2H), 7.14 (t, 1H), 6.75 (d, 1H), | |
| | 6.53 (br s, 1H), 5.08 (t, 1H), 4.29-4.13 (m, 2H), 3.50 (1H + | |
| | H2O) 3.17 (s, 2H), 3.08-3.05 (m, 2H), 2.62-2.59 (m, | |
| | 2H), 1.94-1.86 (m, 4H), 1.56-1.50 (m, 2H), 1.00 (t, 3H) | |
| 7293A | (DMSO): 8.78 (s, 1H), 8.52 (t, 1H), 8.18-.15 (m, 2H), | 526.3 |
| | 7.29-7.25 (m, 2H), 7.13 (t, 1H), 6.74 (d, 1H), 6.33 (br s, | |
| | 1H), 5.08 (t, 1H), 4.28-4.13 (m, 2H), 3.19-3.17 (m, 2H), | |
| | 2.64 (t, 2H), 2.44 (t, 2H), 2.32 (t, 2H), 2.20 (s, 3H), 1.90 | |
| | (quint, 2H), 1.00 (t, 3H) | |
| 7292A | (DMSO): 8.75 (br s, 1H), 8.52 (t, 1H), 8.18-8.15 (m, 2H), | 538.3 |
| | 7.27-7.24 (m, 2H), 7.13 (t, 1H), 6.77-6.72 (m, 2H), 5.08 (t, | |
| | 1H), 4.28-4.13 (m, 3H), 2.78-2.63 (m, 4H), 2.42-2.31 (m, | |
| | 3H), 2.16-2.10 (m, 1H), 1.96-1.86 (m, 2H), 1.65-1.40 (m, | |
| | 1H), 1.00 (t, 3H) | |
| 7291A | (DMSO): 8.75 (br s, 1H), 8.52 (t, 1H), 8.18-8.15 (m, 2H), | 538.3 |
| | 7.27-7.24 (m, 2H), 7.13 (t, 1H), 6.77-6.72 (m, 2H), 5.08 (t, | |
| | 1H), 4.28-4.13 (m, 3H), 2.78-2.63 (m, 4H), 2.42-2.31 (m, | |
| | 3H), 2.16-2.10 (m, 1H), 1.96-1.86 (m, 2H), 1.65-1.40 (m, | |
| | 1H), 1.00 (t, 3H) | |
| 7290A | (DMSO) 8.59-8.48 (m, 2H), 8.16 (s, 1H), 8.11 (s, 1H), | 596.3 |
| | 7.34 (s, 1H), 7.28-7.23 (m, 1H), 7.20-7.11 (m, 1H), 6.79 | |
| | (d, 1H), 5.11-5.01 (m, 1H), 4.31-4.12 (m, 2H), 4.03-3.84 | |
| | (m, 3H), 3.56-3.16 (m, 2H), 3.00-2.84 (m, 1H), 2.74-2.58 | |
| | (m, 5H), 1.95-1.81 (m, 2H), 1.07-0.90 (m, 9H) | |
| 7212A | (DMSO): 8.59-8.49 (m, 2H), 8.20-8.12 (m, 2H), 7.31-7.22 | 552.3 |
| | (m, 2H), 7.15-7.10 (t, 1H), 6.80-6.70 (m, 1H), 6.26 (d, | |
| | 1H), 5.09-5.04 (t, 1H), 4.29-4.14 (m, 2H), 3.74-3.62 (m, | |
| | 1H), 2.70-2.41 (m, 4H), 2.40-2.30 (m, 2H), 2.30-2.19 (m, | |
| | 1H), 2.16-2.05 (m, 1H), 1.98-1.86 (m, 2H), 1.72-1.56 (m, | |
| | 2H), 1.54-1.41 (m, 1H), 1.37-1.21 (m, 1H), 1.01-0.96 (t, | |
| | 3H) | |
| 7211A | (DMSO): 8.96-8.78 (br s, 1H), 8.61.8.49 (m, 1H), | 538.1 |
| | 8.22-8.13 (m, 2H), 7.36-7.23 (m, 2H), 7.15-7.10 (t, 1H), 6.73 | |
| | (d, 1H), 5.10-5.06 (t, 1H), 4.28-4.02 (m, 2H), 3.90-3.70 | |
| | (m, 1H), 3.24-3.08 (m, 4H), 2.92-2.76 (m, 1H), 2.67-2.36 | |
| | (m, 1H + DMSO) 1.99-1.85 (m, 2H), 1.77-1.46 (m, 3H), | |
| | 1.42-1.30 (m, 1H), 1.02-0.97 (t, 3H) | |
| 7146A | (DMSO) 9.23 (br s, 1H), 8.60-8.40 (m, 1H), 8.17 (d, 2H), | 538.0 |
| | 7.38-7.20 (m, 2H), 7.15-7.10 (t, 1H), 6.73 (d, 1H), | |
| | 5.12-5.04 (m, 1H), 4.29-4.12 (m, 2H), 3.90-2.90(m, 9H + | |
| | H2O), 2.05-1.84 (m, 3H), 1.68-1.54 (m, 1H), 1.02-097 (t, | |
| | 3H) | |
| 7145A | (DMSO) 8.80 (s, 1H), 8.53 (s, 1H), 8.17 (d, 1H), 7.30-7.25 | 552.1 |
| | (m, 2H), 7.15-7.10 (t, 1H), 6.73 (d, 1H), 6.65 (s, 1H), | |
| | 5.09-5.05 (t, 1H), 4.23-4.13 (m, 2H), 3.12-2.97 (m, 2H), | |
| | 2.75-2.20 (m, 10H + DMSO), 2.00-1.90 (m 2H), 1.54-1.37 | |
| | (m, 1H) 1.02-0.94 (t, 3H) | |
| 7143A | (CDCl3) 8.52-8.46 (m, 2H), 8.19 (s, 1H), 8.14 (s, 1H), | 536.1 |
| | 7.36-7.33 (m, 2H), 7.18-7.16 (m, 1H), 6.79 (d, 1H), | |
| | 5.10-5.04 (m, 1H), 4.32-4.13 (m, 2H), 4.03 (s, 4H), 3.69 (s, | |
| | 4H), 3.15 (s, 2H), 1.96-1.85 (m, 2H), 1.02-0.97 (t, 3H) | |
| 7142A | (CDCl3) 8.51 (br s, 2H), 8.18 (s, 1H), 8.13 (s, 1H), 7.36 | 523.8 |
| | (s, 1H), 7.28 (d, 1H), 7.18-7.13 (t, 1H), 6.80 (d, 1H), | |
| | 5.10-5.06 (t, 1H), 4.29-4.16 (m, 2H), 3.80-3.10 (m, 8H + H2O), | |
| | 3.08 (s, 2H), 1.93-1.89 (m, 2H), 1.02-9.98 (t, 3H) | |
| 7141A | (CDCl3) 8.60-8.50 (m, 2H), 8.16 (s, 1H), 8.12 (s, 1H), | 568.1 |
| | 7.37-7.13 (m, 3H), 6.86-6.78 (m, 1H), 5.10-5.03 (m, 1H), | |
| | 4.29-4.14 (m, 2H), 4.08-3.09 (m, 6H + H2O), 3.07-2.63 | |
| | (m, 5H), 2.32-2.22(m, 2H), 1.97-1.84 (m, 2H), 1.04-0.94 | |
| | (t, 3H) | |
| 7140A | (CDCl3) 8.60 (s, 1H), 8.58-8.51 (m, 1H), 8.15 (s, 1H), | 554.1 |
| | 8.11 (s, 1H), 7.32 (s, 1H), 7.29-7.13 (m, 2H), 6.86-6.78 | |
| | (m, 1H), 5.11-5.02 (m, 1H), 4.32-3.09 (m, 10H + H2O), | |
| | 3.03-2.82 (m, 2H), 2.77-2.62 (m, 1H), 1.95-1.86 (m, 2H), | |
| | 1.05-0.94 (t, 3H) | |
| 7121A | (CDCl3) 8.51 (br s, 2H), 8.18 (s, 1H), 8.13 (s, 1H), 7.36 | 538.0 |
| | (s, 1H), 7.28 (d, 1H), 7.18-7.13 (t, 1H), 6.80 (d, 1H), | |
| | 5.10-5.06 (m, 1H), 4.25-4.16 (m, 2H), 3.43-3.13 (m, 4H + | |
| | H2O), 2.60-2.55 (m, 2H), 2.39-2.37 (m, 6H), 1.93-1.89 | |
| | (m, 2H), 1.02-9.98 (t, 3H) | |
| 7120A | (DMSO): 8.89 (br, 1H), 8.52-8.50 (m, 1H), 8.17-8.16 (m, | 538.2 |
| | 2H), 7.29-7.24 (m, 2H), 7.12 (t, 1H), 6.92 (br, 1H), 6.73 | |
| | (d, 1H), 5.08 (t, 1H), 4.25-4.13 (m, 3H), 3.40 (br, 1H + | |
| | H2O), 2.77 (br, 1H), 2.63-2.56 (m, 3H), 2.32-2.30 (m, | |
| | 3H), 1.94-1.87 (m, 1H), 1.91 (quint, 2H), 1.53-1.52 (m, | |
| | 1H), 1.00 (t, 3H) | |
| 7119A | (DMSO): 9.40 (br, 1H), 8.52-8.50 (m, 1H), 8.17 (s, 2H), | 524.2 |
| | 7.97 (br, 1H), 7.36-7.28 (m, 2H), 7.12 (t, 1H), 6.73 (d, | |
| | 1H), 5.08 (t, 1H), 4.27-4.18 (m, 3H), 3.70 (br, 1H + H2O), | |
| | 3.42-3.28 (m, 1H), 3.17-3.15 (m, 1H), 3.07 (br m, 1H), | |
| | 2.90 (br m, 1H), 2.74 (br m, 1H), 2.23 (br m, 1H), | |
| | 1.94-1.89 (br m, 2H), 1.69 (m, 1H), 1.00 (t, 3H) | |
| 7062A | (DMSO): 8.53 (t, 1H), 8.44 (s, 1H), 8.18 (d, 1H), 8.13 (d, 1H), 7.35 | 550.2 |
| | (m, 2H), 7.14 (t, 1H), 6.78 (d, 1H), 5.06 (t, 1H), 4.20 (m, 2H), 3.97 (s, | |
| | 4H), 3.20 (s, 4H), 2.51-2.47 (m, 2H + DMSO), 2.02-1.87 (m, 4H), | |
| | 0.99 (t, 3H) | |
| 7061A | (500 MHz, DMSO): 9.02 (br s, 1H), 8.48 (t, 1H), 8.15 (m, | 578.2 |
| | 2H), 7.29 (d, 1H), 7.24 (ddd, 1H), 7.10 (t, 2H), 6.71 (dt, | |
| | 1H), 5.07 (t, 1H), 4.18 (m, 2H), 3.96 (m, 1H), 3.20 (s, | |
| | 2H), 3.12 (s, 2H), 2.56 (t, 2H), 2.36 (ddt, 2H), 2.04 (t, | |
| | 2H), 1.92 (m, 4H) | |
| 6949A | (MeOD): 7.44 (s, 1H), 7.29 (s, 1H), 7.24-7.10 (m, 3H), | 549.1 |
| | 6.97-6.93(m, 1H), 6.85 (br s, 1H), 4.80-4.68 (m, 1H), 4.37 | |
| | (s, 2H), 4.12-4.08 (m 1H), 3.78 (s, 2H), 3.67 (s, 2H), | |
| | 3.05-292 (m, 2H), 2.66-2.60 (m, 2H), 2.34-2.28 (m, 2H), | |
| | 2.18-2.08 (m, 2H), 1.64-1.57 (m, 3H) | |
| 6948A | (MeOD): 7.35 (d, 1H), 7.29 (s, 1H), 7.16 (brs, 2H), 6.90-6.79 | 597.2 |
| | (m, 2H), 4.61-4.57 (t, 1H), 4.34 (d, 2H), 4.13-4.08 (t, | |
| | 1H), 3.87 (s, 2H), 3.76 (s, 2H), 3.10-3.02 (m, 2H), | |
| | 2.70-2.58 (m 2H), 2.34-2.30 (t, 2H), 2.20-2.08 (m, 2H), | |
| | 2.07-1.95 (m, 2H), 1.11-1.05 (t, 3H) | |
| 6754B | (MeOD): 8.17 (t, 2H), 7.40 (s, 1H), 7.22 (d, 1H), 7.08 (t, 1H), 6.66 (d, | 592.2 |
| | 1H), 5.07 (t, 1H), 4.18 (q, 2H), 3.93 (m, 1H), 3.13 (d, 5H), 2.31 (m, | |
| | 4H), 1.90 (m, 4H), 0.99 (t, 3H), 0.93 (s, 6H) | |
| 6754A | (MeOD): 8.17 (t, 2H), 7.40 (s, 1H), 7.22 (d, 1H), 7.08 (t, | 592.2 |
| | 1H), 6.66 (d, 1H), 5.07 (t, 1H), 4.18 (q, 2H), 3.93 (m, 1H), | |
| | 3.13 (d, 5H), 2.31 (m, 4H), 1.90 (m, 4H), 0.99 (t, 3H), | |
| | 0.93 (s, 6H) | |
| 6681B | (DMSO): 9.31 (s, 1H), 8.49 (t, 1H), 8.15 (m, 2H), 7.48 (d, | 550.2 |
| | 1H), 7.32 (s, 1H), 7.26 (d, 1H), 7.11 (t, 1H), 6.72 (d, 1H), | |
| | 5.08 (dd, 1H), 4.20 (m, 2H), 4.01 (dt, 1H), 3.89 (s, 2H), | |
| | 3.96 (s, 2H), 2.56 (m, 2H), 2.10 (m, 2H), 1.91 (m, 2H), | |
| | 0.99 (t, 3H) | |
| 6681A | (MeOD): 7.96 (s, 1H), 7.88 (s, 1H), 7.18 (m, 3H), 6.85 | 550.2 |
| | (m, 1H), 5.08 (t, 1H), 4.33 (s, 2H), 4.07 (t, 1H), 3.38 (s, | |
| | 2H), 3.28 (s, 2H), 3.06 (s, 2H), 2.53 (m, 2H), 2.00 (m, | |
| | 4H), 1.11 (t, 3H) | |
| 6680A | MeOD): 7.96 (s, 1H), 7.87 (s, 1H), 7.18 (m, 3H), 6.85 (m, | 578.2 |
| | 1H), 5.08 (t, 1H), 4.33 (s, 2H), 5.08 (t, 1H), 3.19 (s, 2H), | |
| | 2.47 (m, 6H), 2.13 (t, 2H), 2.00 (m, 4H), 1.62 (m, 2H), | |
| | 1.06 (t, 3H) | |
| 6679B | (MeOD): 7.99 (s, 1H), 7.90 (s, 1H), 7.22 (m, 3H), 6.87 | 590.2 |
| | (m, 1H), 5.10 (t, 1H), 4.35 (s, 2H), 4.11 (t, 1H), 3.21 (m, | |
| | 2H + MeOH), 3.19 (m, 3H), 2.94 (m, 1H), 2.53 (m, 2H), | |
| | 2.27 (m, 2H), 2.00 (m, 6H), 1.11 (t, 3H) | |
| 6679A | (MeOD): 7.99 (s, 1H), 7.90 (s, 1H), 7.22 (m, 3H), 6.87 | 590.2 |
| | (m, 1H), 5.10 (t, 1H), 4.35 (s, 2H), 4.11 (t, 1H), 3.21 (m, | |
| | 2H + MeOH), 3.19 (m, 3H), 2.94 (m, 1H), 2.53 (m, 2H), | |
| | 2.27 (m, 2H), 2.00 (m, 6H), 1.11 (t, 3H) | |
| 5548A | (DMSO): 8.55 (t, 1H), 8.26 (d, 1H), 8.17 (dd, 2H), 7.22 | 563.4 |
| | (m, 1H), 7.10-7.05 (m, 3H), 5.07 (t, 1H), 4.27-4.22 (m, | |
| | 2H), 3.94 (q, 2H), 3.30 (s, 2H), 3.21 (s, 2H), 3.12 (s, 2H), | |
| | 2.57 (t, 2H), 2.36-2.30 (m, 2H), 2.13 (t, 2H), 2.00-1.85 (m, | |
| | 4H), 1.00 (t, 3H) | |
| 5533A | (MeOD): 8.00 (s, 1H), 7.89 (s, 1H), 7.79-7.67 (m, 2H), | 535.4 |
| | 7.47-7.67 (m, 2H), 5.10-5.06 (t, 1H), 4.76-4.68 (q, 1H), | |
| | 4.44 (s, 2H), 4.07-4.02 (t, 1H), 3.95-3.89 (t, 1H), | |
| | 3.85-3.79 (m, 1H), 3.63-3.45 (m, 2H), 3.08-2.92 (m, 1H), | |
| | 2.49-2.32 (m, 2H), 2.24-2.08 (m, 2H), 2.07-1.93 (m, 2H), | |
| | 1.11-1.06 (t, 3H) | |
| 5532A | (MeOD): 7.98 (s, 1H), 7.91 (s, 1H), 7.56-7.47 (, 2H), 7.40 | 561.4 |
| | (brs, 2H), 5.10-5.06 (t, 1H), 4.51-4.28 (m, 6H), 3.98-3.86 | |
| | (m, 2H), 3.78-3.61 (m, 2H), 3.55-3.42 (m, 1H), 3.02-2.71 | |
| | (m, 1H), 2.50-2.27 (m, 2H), 2.8-1.93 (m 4H), 1.12-1.07 (t, | |
| | 3H) | |
| 5531A | (MeOD): 7.98 (s, 1H), 7.92 (s, 1H), 7.58-7.47 (m, 2H), | 549.4 |
| | 7.40 (brs, 2H), 5.10-5.06 (t, 1H), 4.56-4.31 (m, 6H), | |
| | 4.30-4.14 (m, 4H), 3.14-3.10 (t, 2H), 2.37-2.33 (t, 2H), | |
| | 2.10-1.96 (m, 2H), 1.80-1.66 (s, 2H), 1.12-1.07 (t, 3H) | |
| 5530A | (MeOD): 7.97 (s, 1H), 7.92 (s, 1H), 7.56-7.47 (m, 2H), | 563.3 |
| | 7.40 (brs, 2H), 5.10-5.05 (t, 1H), 4.49-4.21 (m, 10H), 3.10 | |
| | (s, 2H), 2.07-1.96 (m, 2H), 1.20-1.03 (m, 9H) | |
| 5529A | (MeOD): 7.98 (s, 1H), 7.94 (s, 1H), 7.59-7.48 (m, 2H), | 549.4 |
| | 7.45-7.34 (m, 2H), 5.11-5.07 (t, 1H), 4.52-4.34 (m, 2H), | |
| | 4.32-4.11 (m, 3H), 3.76-3.61 (m, 1H), 3.08-3.04 (t, 2H), | |
| | 2.71-2.53 (m 2H), 2.51-2.32 (m, 4H), 2.08-1.99 (m, 2H), | |
| | 1.14-1.09 (t, 3H) | |
| 5527A | (CDCl₃ + MeOD): 7.84 (d, 1H), 7.61-7.56 (m, 3H), 7.26 | 535.2 |
| | (m, 2H + CHCl₃), 5.22 (t, 1H), 4.37-4.28 (m, 3H), 4.04 (s, | |
| | 2H), 3.98 (s, 2H), 3.53 (s, 2H + MeOH), 2.64 (m, 2H), | |
| | 2.26 (m, 2H), 1.95 (quint, 2H), 0.94 (t, 3H) | |
| 5526A | (CDCl₃): 7.94 (d, 1H), 7.62 (m, 3H), 7.26 (m, 2H + | 563.2 |
| | CHCl3), 6.97 (br, 1H), 5.35 (t, 1H), 4.37 (m, 3H), 3.49-3.38 | |
| | (m, 5H), 2.65-2.59 (m, 4H), 2.33-2.17 (m, 4H), 2.03 | |
| | (m, 2H), 1.64 (m, 2H), 0.99 (t, 3H) | |
| 5525A | (CDCl₃ + MeOD): 7.94 (d, 1H), 7.70-7.63 (m, 3H), 7.39-7.35 | 577.3 |
| | (m, 2H + CHCl3), 5.31 (t, 1H), 4.46 (s, 2H), 4.38 (m, | |
| | 1H), 3.96 (s, 2H), 3.88 (s, 2H), 2.88 (s, 2H), 2.70 (m, 2H), | |
| | 2.31 (m, 2H), 2.03 (quint, 2H), 1.50 (s, 6H), 1.03 (t, 3H) | |
| 5524A | (MeOD): 7.95 (d, 1H), 7.87 (d, 1H), 7.68-7.63 (m, 2H), | 563.4 |
| | 7.38-7.35 (m, 2H), 5.07 (t, 1H), 4.47-4.38 (m, 3H), 3.67 | |
| | (quint, 1H), 3.03 (t, 2H), 2.62-2.54 (m, 2H), 2.48-2.30 (m, | |
| | 4H), 2.26-2.15 (m, 4H), 1.97 (quint, 2H), 1.08 (t, 3H) | |
| 5523A | (MeOD): 7.99 (dd, 1H), 7.89 (t, 1H), 7.84-7.79 (m, 2H), | 549.5 |
| | 7.45-7.38 (m, 2H), 5.09 (t, 1H), 4.88-4.38 (s, 4H), | |
| | 4.08-4.06 (m, 3H), 3.34-3.21 (m, 2H), 2.65-2.32 (m, 2H), | |
| | 2.27-2.12 (m, 3H), 2.07-1.90 (m, 3H), 1.34 (t, 3H) | |
| 5522A | (MeOD): 7.97 (d, 1H), 7.89 (d, 1H), 7.70-7.62 (m, 2H), | 563.4 |
| | 7.39-7.37 (m, 2H), 5.08 (t, 1H), 4.42 (s, 2H), 4.16-4.08 | |
| | (m, 4H), 3.37 (d, 2H), 3.31-3.21 (m, 3H + MeOH), | |
| | 2.65-2.38 (m, 4H), 2.12-1.97 (m, 4H), 1.08 (t, 3H) | |
| 5456A | (500 MHz, MeOD) δ 7.90-8.10 (m, 2H), 7.20-7.50 (m, | 466.0 |
| | 4H), 5.10-5.30 (m, 2H), 4.30-4.60 (m, 2H), 3.90-4.10 (m, | |
| | 2H), 3.50-3.7 (m, 1H), 2.70-2.90 (m, 1H), 2.40-2.70 (m, | |
| | 4H), 2.2-2.3 (m, 2H), 2.00-2.10 (m, 3H), 1.30-1.50 (m, | |
| | 2H), 1.00-1.20 (m, 3H) | |
| 5454A | (DMSO): 9.08 (br s, 1H), 8.71 (m, 1H), 8.16 (m, 2H), 7.80 | 495.0 |
| | (s, 1H), 7.73 (m, 1H), 7.39 (m, 2H), 5.05 (m, 1H), 4.65 | |
| | (m, 1H), 4.32 (m, 2H), 3.92 (br s, 2H), 3.51 (br s, 2H), | |
| | 3.29 (br s, 2H), 1.90 (m, 2H), 0.99 (t, 3H) | |
| 5453A | (MeOD): 7.98 (d, 1H), 7.92 (d, 1H), 7.49 (m, 2H), 7.38 | 535.4 |
| | (m, 2H), 5.08 (t, 1H), 4.36 (m, 6H), 3.39 (m, 4H), 2.69 (t, | |
| | 2H), 2.19 (t, 2H), 2.01 (m, 2H), 1.08 (t, 3H) | |
| 5452A | (MeOD): 7.96 (d, 1H), 7.88 (d, 1H), 7.18 (m, 3H), 6.85(m, 1H), 5.08 | 564.5 |
| | (t, 1H), 4.33 (s, 2H), 4.08 (m, 1H), 3.31 (2H + MeOH), 3.20 (s, 2H), | |
| | 2.68 (m, 2H), 2.53 (m, 2H), 2.21 (m, 2H), 2.00 (m, 4H), 1.08 (t, 3H) | |
| 5442A | (DMSO): 8.50 (m, 1H), 8.18 (m, 2H), 7.28 (m, 2H), 7.10 | 524.2 |
| | (t, 1H), 6.71 (d, 1H), 5.06 (m, 1H), 4.23-4.12 (m, 3H), | |
| | 3.42 (br s, 2H), 2.93 (br s, 2H), 2.62 (br t, 2H), 2.13 (br t, | |
| | 2H), 1.88 (m, 2H), 0.98 (t, 3H) | |
| 5441A | (DMSO): 10.58 (s, 1H), 8.72 (m, 1H), 8.19 (m, 2H), 7.87 | 574.9 |
| | (s, 1H), 7.80 (d, 1H), 7.69 (s, 1H), 7.43-7.40 (m, 2H), 5.19 | |
| | (m, 1H), 4.96 (m, 1H), 4.32 (m, 2H), 3.67 (br t, 2H), 3.24 | |
| | (br t, 2H), 2.67 (br t, 2H), 2.19 (br t, 2H), 1.91 (m, 2H), | |
| | 1.00 (t, 3H) | |
| 5430A | (DMSO): 8.88 (d, 1H), 8.68 (t, 1H), 8.17 (m, 2H), 7.74 (s, | 523.0 |
| | 1H), 7.70 (d, 1H), 7.40-7.32 (m, 2H), 5.08 (m, 1H), | |
| | 4.50-4.23 (m, 3H), 3.56 (t, 2H), 2.95 (br, 2H), 2.40 (m, 2H), | |
| | 2.12 (t, 2H), 1.90 (m, 2H), 1.49 (m, 2H), 0.99 (t, 3H) | |
| 5427A | (DMSO): 8.83 (d, 1H), 8.72 (t, 1H), 8.17 (m, 2H), 7.76 (s, | 509.0 |
| | 1H), 7.71 (d, 1H), 7.40-7.33 (m, 2H), 5.08 (m, 1H), 4.48 | |
| | (m, 1H), 4.31 (m 2H), 3.52 (br t, 2H), 3.00 (br, 2H), 2.62 | |
| | (br t, 2H), 2.09 (br t, 1H), 1.89 (m, 2H), 0.99 (t, 3H) | |
| 5414B | (MeOD): 7.96 (s, 1H), 7.89 (s, 1H), 7.70-7.65 (m, 2H), | 549.0 |
| | 7.40 (m, 2H), 5.08 (t, 1H), 4.43-4.36 (m, 3H), 4.27 (s, | |
| | 2H), 4.15 (s, 2H), 3.36 (t, 2H + MeOH), 2.76 (m, 2H), | |
| | 2.51-2.41 (m, 4H), 2.01 (t, 2H), 1.09 (t, 3H) | |

The T3SS inhibitor compounds described herein are organic compounds that can also be synthesized to order by commercial suppliers such as ChemBridge Corporation (San Diego, CA, USA), Life Chemicals Inc. (Burlington, ON, Canada), and Timtec LLC (Newark, DE, USA).

Unless otherwise indicated, it is understood that description of the use of a T3SS inhibitor compound in a composition or method also encompasses the embodiment wherein a combination of two or more T3SS inhibitor compounds are employed as the source of T3SS inhibitory activity in a composition or method of the invention.

Pharmaceutical compositions according to the invention comprise a T3SS inhibitor compound as described herein, or a pharmaceutically acceptable salt thereof, as the “active ingredient” and a pharmaceutically acceptable carrier (or “vehicle”), which may be a liquid, solid, or semi-solid compound. A “pharmaceutically acceptable” compound or composition means that the compound or composition is not biologically, chemically, or in any other way, incompatible with body chemistry and metabolism and also does not adversely affect the activity of the T3SS inhibitor or any other component that may be present in a composition in such a way that would compromise the desired therapeutic and/or preventative benefit to a patient. Pharmaceutically acceptable carriers useful in the invention include those that are known in the art of preparation of pharmaceutical compositions and include, without limitation, water, physiological pH buffers, physiologically compatible salt solutions (e.g., phosphate buffered saline), and isotonic solutions. Pharmaceutical compositions of the invention may also comprise one or more excipients, i.e., compounds or compositions that contribute or enhance a desirable property in a composition other than the active ingredient.

Various aspects of formulating pharmaceutical compositions, including examples of various excipients, dosages, dosage forms, modes of administration, and the like are known to those skilled in the art of pharmaceutical compositions and also available in standard pharmaceutical texts, such as Remington's Pharmaceutical Sciences, 18th edition, Alfonso R. Gennaro, ed. (Mack Publishing Co., Easton, PA 1990), Remington: The Science and Practice of Pharmacy, Volumes 1 & 2, 19th edition, Alfonso R. Gennaro, ed., (Mack Publishing Co., Easton, PA 1995), or other standard texts on preparation of pharmaceutical compositions.

Pharmaceutical compositions may be in any of a variety of dosage forms particularly suited for an intended mode of administration. Such dosage forms, include, but are not limited to, aqueous solutions, suspensions, syrups, elixirs, tablets, lozenges, pills, capsules, powders, films, suppositories, and powders, including inhalable formulations. Preferably, the pharmaceutical composition is in a unit dosage form suitable for single administration of a precise dosage, which may be a fraction or a multiple of a dose that is calculated to produce effective inhibition of T3SS.

A composition comprising a T3SS inhibitor compound (or combination of T3SS inhibitors) described herein may optionally possess a second active ingredient (also referred to as “second agent”, “second active agent”) that provides one or more other desirable therapeutic or prophylactic activities other than T3SS inhibitory activity. Such a second agent useful in compositions of the invention includes, but is not limited to, an antibiotic, an antibody, an antiviral agent, an anticancer agent, an analgesic (e.g., a nonsteroidal anti-inflammatory drug (NSAID), acetaminophen, an opioid, a COX-2 inhibitor), an immunostimulatory agent (e.g., a cytokine or a synthetic immunostimulatory organic molecule), a hormone (natural, synthetic, or semi-synthetic), a central nervous system (CNS) stimulant, an antiemetic agent, an anti-histamine, an erythropoietin, a complement stimulating agent, a sedative, a muscle relaxant agent, an anesthetic agent, an anticonvulsive agent, an antidepressant, an antipsychotic agent, and combinations thereof.

Pharmaceutical compositions as described herein may be administered to humans and other animals in a manner similar to that used for other known therapeutic or prophylactic agents, and particularly as used for therapeutic aromatic or multi-ring antibiotics. The dosage to be administered to an individual and the mode of administration will depend on a variety of factors including age, weight, sex, condition of the patient, and genetic factors, and will ultimately be decided by an attending qualified healthcare provider.

Pharmaceutically acceptable salts of T3SS inhibitor compounds described herein include those derived from pharmaceutically acceptable inorganic and organic acids and bases. Examples of suitable acids include hydrochloric, hydrobromic, sulfuric, nitric, perchloric, fumaric, maleic, malic, palmoic, phosphoric, glycolic, lactic, salicylic, succinic, toluene-p-sulfonic, tartaric, acetic, citric, methanesulfonic, formic, benzoic, malonic, naphthalene-2-sulfonic, tannic, carboxymethyl cellulose, polylactic, polyglycolic, and benzenesulfonic acids.

The invention may also envision the “quaternization” of any basic nitrogen-containing groups of a compound described herein, provided such quaternization does not destroy the ability of the compound to inhibit T3SS. Such quaternization may be especially desirable to enhance solubility. Any basic nitrogen can be quaternized with any of a variety of compounds, including but not limited to, lower (e.g., C₁-C₄)alkyl halides (e.g., methyl, ethyl, propyl and butyl chlorides, bromides, and iodides); dialkyl sulfates (e.g., dimethyl, diethyl, dibutyl and diamyl sulfates); long chain halides (e.g., decyl, lauryl, myristyl and stearyl chlorides, bromides and iodides); and aralkyl halides (e.g., benzyl and phenethyl bromides).

For solid compositions, conventional nontoxic solid carriers may be used including, but not limited to, mannitol, lactose, starch, magnesium stearate, sodium saccharin, talc, cellulose, glucose, sucrose, and magnesium carbonate.

Pharmaceutical compositions may be formulated for administration to a patient by any of a variety of parenteral and non-parenteral routes or modes. Such routes include, without limitation, intravenous, intramuscular, intra-articular, intraperitoneal, intracranial, paravertebral, periarticular, periostal, subcutaneous, intracutaneous, intrasynovial, intrasternal, intrathecal, intralesional, intratracheal, sublingual, pulmonary, topical, rectal, nasal, buccal, vaginal, or via an implanted reservoir. Implanted reservoirs may function by mechanical, osmotic, or other means. Generally and particularly when administration is via an intravenous, intra-arterial, or intramuscular route, a pharmaceutical composition may be given as a bolus, as two or more doses separated in time, or as a constant or non-linear flow infusion.

A pharmaceutical composition may be in the form of a sterile injectable preparation, e.g., as a sterile injectable aqueous solution or an oleaginous suspension. Such preparations may be formulated according to techniques known in the art using suitable dispersing or wetting agents (e.g., polyoxyethylene 20 sorbitan monooleate (also referred to as “polysorbate 80”); TWEEN® 80, ICI Americas, Inc., Bridgewater, New Jersey) and suspending agents. Among the acceptable vehicles and solvents that may be employed for injectable formulations are mannitol, water, Ringer's solution, isotonic sodium chloride solution, and a 1,3-butanediol solution. In addition, sterile, fixed oils may be conventionally employed as a solvent or suspending medium. For this purpose, a bland fixed oil may be employed including synthetic mono- or diglycerides. Fatty acids, such as oleic acid and its glyceride derivatives are useful in the preparation of injectables, as are natural pharmaceutically-acceptable oils, including olive oil or castor oil, especially in their polyoxyethylated versions.

A T3SS inhibitor described herein may be formulated in any of a variety of orally administrable dosage forms including, but not limited to, capsules, tablets, caplets, pills, films, aqueous solutions, oleaginous suspensions, syrups, or elixirs. In the case of tablets for oral use, carriers, which are commonly used include lactose and corn starch. Lubricating agents, such as magnesium stearate, are also typically added. For oral administration in a capsule form, useful diluents include lactose and dried cornstarch. Capsules, tablets, pills, films, lozenges, and caplets may be formulated for delayed or sustained release.

Tablets and other solid or semi-solid formulations may be prepared that rapidly disintegrate or dissolve in an individual's mouth. Such rapid disintegration or rapid dissolving formulations may eliminate or greatly reduce the use of exogenous water as a swallowing aid. Furthermore, rapid disintegration or rapid dissolve formulations are also particularly useful in treating individuals with swallowing difficulties. For such formulations, a small volume of saliva is usually sufficient to result in tablet disintegration in the oral cavity. The active ingredient (a T3SS inhibitor described herein) can then be absorbed partially or entirely into the circulation from blood vessels underlying the oral mucosa (e.g., sublingual and/or buccal mucosa), or it can be swallowed as a solution to be absorbed from the gastrointestinal tract.

When aqueous suspensions are to be administered orally, whether for absorption by the oral mucosa or absorption via the gut (stomach and intestines), a composition comprising a T3SS inhibitor may be advantageously combined with emulsifying and/or suspending agents. Such compositions may be in the form of a liquid, dissolvable film, dissolvable solid (e.g., lozenge), or semi-solid (chewable and digestible). If desired, such orally administrable compositions may also contain one or more other excipients, such as a sweetener, a flavoring agent, a taste-masking agent, a coloring agent, and combinations thereof.

The pharmaceutical compositions comprising a T3SS inhibitor as described herein may also be formulated as suppositories for vaginal or rectal administration. Such compositions can be prepared by mixing a T3SS inhibitor compound as described herein with a suitable, non-irritating excipient that is solid at room temperature but liquid at body temperature and, therefore, will melt in the appropriate body space to release the T3SS inhibitor and any other desired component of the composition. Excipients that are particularly useful in such compositions include, but are not limited to, cocoa butter, beeswax, and polyethylene glycols.

Topical administration of a T3SS inhibitor may be useful when the desired treatment involves areas or organs accessible by topical application, such as the epidermis, surface wounds, or areas made accessible during surgery. Carriers for topical administration of a T3SS inhibitor described herein include, but are not limited to, mineral oil, liquid petroleum, white petroleum, propylene glycol, polyoxyethylene polyoxypropylene compounds, emulsifying wax, and water. Alternatively, a topical composition comprising a T3SS inhibitor as described herein may be formulated with a suitable lotion or cream that contains the inhibitor suspended or dissolved in a suitable carrier to promote absorption of the inhibitor by the upper dermal layers without significant penetration to the lower dermal layers and underlying vasculature. Carriers that are particularly suited for topical administration include, but are not limited to, mineral oil, sorbitan monostearate, polysorbate 60, cetyl esters wax, cetearyl alcohol, 2-octyldodecanol, benzyl alcohol, and water. A T3SS inhibitor may also be formulated for topical application as a jelly, gel, or emollient. Topical administration may also be accomplished via a dermal patch.

Persons skilled in the field of topical and transdermal formulations are aware that selection and formulation of various ingredients, such as absorption enhancers, emollients, and other agents, can provide a composition that is particularly suited for topical administration (i.e., staying predominantly on the surface or upper dermal layers with minimal or no absorption by lower dermal layers and underlying vasculature) or transdermal administration (absorption across the upper dermal layers and penetrating to the lower dermal layers and underlying vasculature).

Pharmaceutical compositions comprising a T3SS inhibitor as described herein may be formulated for nasal administrations, in which case absorption may occur via the mucous membranes of the nasal passages or the lungs. Such modes of administration typically require that the composition be provided in the form of a powder, solution, or liquid suspension, which is then mixed with a gas (e.g., air, oxygen, nitrogen, or a combination thereof) so as to generate an aerosol or suspension of droplets or particles. Inhalable powder compositions preferably employ a low or non-irritating powder carrier, such as melezitose (melicitose). Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. A pharmaceutical composition comprising a T3SS inhibitor described herein for administration via the nasal passages or lungs may be particularly effective in treating lung infections, such as hospital-acquired pneumonia (HAP).

Pharmaceutical compositions described herein may be packaged in a variety of ways appropriate to the dosage form and mode of administration. These include but are not limited to vials, bottles, cans, packets, ampoules, cartons, flexible containers, inhalers, and nebulizers. Such compositions may be packaged for single or multiple administrations from the same container. Kits may be provided comprising a composition, preferably as a dry powder or lyophilized form, comprising a T3SS inhibitor and preferably an appropriate diluent, which is combined with the dry or lyophilized composition shortly before administration as explained in the accompanying instructions of use. Pharmaceutical composition may also be packaged in single use pre-filled syringes or in cartridges for auto-injectors and needleless jet injectors. Multi-use packaging may require the addition of antimicrobial agents such as phenol, benzyl alcohol, meta-cresol, methyl paraben, propyl paraben, benzalconium chloride, and benzethonium chloride, at concentrations that will prevent the growth of bacteria, fungi, and the like, but that are non-toxic when administered to a patient.

Consistent with good manufacturing practices, which are in current use in the pharmaceutical industry and which are well known to the skilled practitioner, all components contacting or comprising a pharmaceutical composition must be sterile and periodically tested for sterility in accordance with industry norms. Methods for sterilization include ultrafiltration, autoclaving, dry and wet heating, exposure to gases such as ethylene oxide, exposure to liquids, such as oxidizing agents, including sodium hypochlorite (bleach), exposure to high energy electromagnetic radiation (e.g., ultraviolet light, x-rays, gamma rays, ionizing radiation). Choice of method of sterilization will be made by the skilled practitioner with the goal of effecting the most efficient sterilization that does not significantly alter a desired biological function of the T3SS inhibitor or other component of the composition.

Additional embodiments and features of the invention will be apparent from the following non-limiting examples.

### EXAMPLES

### Example 1. Materials and Methods for Characterization of T3SS Inhibitors

### Strains, Plasmids, and Growth Media.

Bacterial strains and plasmids used for assays are described in Table 4, below. All P. aeruginosa strains were derivatives of PAO1 (Holloway, et al., 1979, Microbiol. Rev., 43:73-102), PAK (Bradley, D. E., 1974, Virology, 58:149-63), or PA99 (Feltman et al., 2004, Microbiology 147:2659-2669). E. coli TOP10 (Invitrogen), E. coli DB3.1 (GATEWAY® host, Invitrogen), E. coli SM10 (de Lorenzo and Timmis, 1994, Methods Enzymol., 235:386-405), and E. coli S17-1 (ATCC 47055) were used as hosts for molecular cloning. Luria-Bertani (LB) medium (liquid and agar) was purchased from Difco. LB was supplemented with 30 μg/ml gentamicin (LBG) with or without 1 mM isopropyl-β-D-thiogalactopyranoside (IPTG) and 5 mM EGTA (LBGI and LBGIE, respectively).

TABLE 4
Strains and Plasmids

| | | Reference |
|---|---|---|
| Strain | Genotype/Features | or Source |
| P. aeruginosa: | | |
| MDM852 | PAO1::pGSV3-‘exoT’-luxCDABE | (1) |
| MDM1710 | PAO397 exoS-blaM | (2) |
| MDM1746 | PAO1 exoS-blaM | This study |
| MDM2446 | PAO1 exoS-blaM pscF(R75H) | This study |
| MDM1838 | PAO1 ΔpscF exoS-blaM | (2) |
| MDM1566 | PA99 | (3) |
| MDM1561 | PA99U | (3) |

(1) Aiello, et al., 2010, Antimicrob. Agents Chemother., 54: 1988-99.
(2) Bowlin, et al., 2014, Antimicrob. Agents Chemother., 58: 2211-2220.
(3) Shaver and Hauser, 2004. Infect Immun 72: 6969-6977.
Plasmid pGSV3-Lux was kindly provided by Dr. Donald Woods (U. Calgary) (4) Moore et al., Infect Immun, 2004; 72: 4172-4187.

Luciferase Transcriptional Reporter Assay of T3SS-Mediated Secretion with a Luminescent Signal.

A transcriptional fusion of the Photorhabdus luminescens lux operon (luxCDABE) to P. aeruginosa PAO1 T3SS effector gene exoT (PA0044) was constructed and used to build strain MDM852 as described previously (Aiello et al., 2010, Antimicrob Agents Chemother, 54:1988-1999). Induction of T3SS operons by Ca⁺⁺ depletion due to EGTA addition results in secretion of the T3SS negative regulator ExsE and the production of high levels of T3SS components including the luxCDABE gene products, which results in luminescence that is easily detected by a microplate reader. Inhibition of T3SS-mediated secretion of the negative regulator ExsE by a small molecule inhibitor causes reduced production of luxCDABE gene products and reduced luminescence. For inhibitor IC₅₀ testing, compounds were added to a 96-well microplate in a concentration dilution series. Reporter strain MDM852 was grown at 37° C. in LBG to OD₆₀₀ ~0.025-0.05, transferred into the microplate (50 uL/well) containing test compounds and EGTA (5 uL of 0.1 M stock solution), which was covered with a translucent gas-permeable seal (Abgene, Inc., Cat. No. AB-0718). Control wells contained cells with fully induced T3SS (EGTA and test compound diluent DMSO, columns 1 and 2) and uninduced T3SS (DMSO only, columns 11 and 12). Plates were incubated at room temperature for 300 min. Then, luminescence was measured in an Envision Multilabel microplate reader (PerkinElmer). The screening window coefficient, Z′-factor (see Zhang, et al., 1999, J. Biomol. Screen., 4:67-73), defined as the ratio of the positive and negative control separation band to the signal dynamic range of the assay, averaged 0.7 for the assay. An IC₅₀ (the concentration of inhibitor causing 50% inhibition of T3SS-mediated secretion) was calculated for each test article and used to rank inhibitors by potency.

Effector-β-Lactamase (βLA) Reporter Assay of T3SS-Mediated Secretion with an Absorbency Signal.

A gene encoding the exoS promoter/regulatory region and an ExoS-β-lactamase (βLA) fusion protein (comprised of the full length P. aeruginosa T3SS effector ExoS fused in reading frame to the TEM-1 β-lactamase gene lacking secretion signal codons) was constructed by splicing by overlap extension PCR (SOE-PCR) (Choi and Schweizer, 2005, BMC Microbiol., 5:30), sequence confirmed, cloned into miniCTX and introduced into P. aeruginosa strain PAO1 to generate strains MDM1746 and MDM1838 as previously described for strain MDM1710 (Bowlin et al., 2014, Antimicrob Agents Chemother., 58:2211-2220). Secretion of ExoS-β-lactamase fusion protein following EGTA addition to induce the T3SS operons was detected by measuring the hydrolysis of the chromogenic β-lactamase substrate nitrocefin in clear 96-well microplates in a modification of a previously described assay (Lee, et al., 2007, Infect. Immun., 75:1089-98). Cells of strain MDM1746, or negative control MDM1838 (which fails to carry out T3SS secretion due to ΔpscF), were sub-cultured in the morning from overnight growths in LBG into 0.1 ml of LBGE with or without test compounds and grown for 150 min. Nitrocefin (100 μg/mL final) was added, and A490 measurements taken every minute for 15 min in a Victor³V 1420 Multilabel HTS Counter (PerkinElmer). Slopes were calculated as a relative measure of the quantity of the effector-βLA fusion protein secreted and were absolutely dependent on induction with EGTA, and the presence of a functional pscF gene in the P. aeruginosa cells. Typical signal: background ratios were 6-10. An IC₅₀ (the concentration of inhibitor causing 50% inhibition of T3SS-mediated secretion) was calculated for each test article and used to rank inhibitors by potency.

P. aeruginosa ExoU-Dependent CHO Cell Killing. Assay for T3SS-Mediated Translocation:

Rescue of CHO cells from T3SS-mediated cytotoxicity of translocated effector protein ExoU by P. aeruginosa strain MDM1561 (PA99U) was measured using a lactate dehydrogenase (LDH) release assay as previously reported ((Lee, et al., 2005, Infect. Immun., 73:1695-705) except that infection with P. aeruginosa was carried out for 2 hr in the absence of gentamicin. Percent cytotoxicity (% LDH release) was calculated relative to that of the uninfected control, which was set at 0% LDH release, and that of cells infected with P. aeruginosa strain MDM1561 (PA99U) in the absence of test inhibitory compound (100% LDH release). LDH released from uninhibited, infected cells reached at least 80% of the value obtained from complete lysis with 1% Triton X-100 in the 2 hr timeframe of this experiment. Pseudolipasin, which acts by direct inhibition of the ExoU phospholipase, was used as control inhibitor in some experiments (Lee, et al, 2007, Infect. Immun., 75:1089-98). An EC₅₀ (the concentration of inhibitor causing 50% inhibition of T3SS-mediated translocation) was calculated for each test article and used to rank inhibitors by potency.

### Minimum Inhibitory Concentration (MIC) Assay.

MIC determination was done by the broth microdilution method described in the CLSI (formerly NCCLS) guidelines and expressed in μM to facilitate comparisons with IC₅₀ and CC₅₀ values. See, NCCLS, Approved standard M7-A4: Methods for dilution antimicrobial susceptibility tests for bacteria that grow aerobically, 4^{th} ed. National Committee for Clinical Laboratory Standards, Wayne, PA (1997). T3SS inhibitors do not affect the growth of P. aeruginosa during culture in vitro; therefore, compounds with detectable MIC values were discarded.

### Determination of Mammalian Cytotoxicity.

The cytotoxic concentration (CC₅₀) of compound versus cultured mammalian cells (HeLa, ATCC CCL-2; American Type Culture Collection, Manassas, VA) was determined as the concentration of compound that inhibits 50% of the conversion of MTS to formazan (Marshall, et al., 1995, Growth Regul. 5:69-84). Briefly, 96-well plates were seeded with HeLa cells at a density of 4×10³ per well in VP-SFM medium without serum (Frazzati-Gallina, et al., 2001, J. Biotechnol., 92:67-72), in the presence or absence of serial dilutions of a compound dissolved in DMSO. Following incubation for 3 days at 37° C. in VP-SFM, cell viability was measured with the vital tetrazolium salt stain 3-(4,5-dimethylthiazol-2-yl)-2,5 diphenyltetrazolium bromide according to the manufacturer's instructions (Promega, Madison, Wisconsin). Values were determined in triplicate using dilutions of inhibitory compound from 100 μM to 0.2 μM.

Liver microsome stability. To examine the potential for first-pass metabolism of analogs in the liver, we will measure the stability of analogs in the presence of mouse and human liver microsome preparations (XenoTech). The stability of compounds in the presence of NADPH will be measured using the method of Kuhnz et al. (48) Analogs that are stable in the presence of mouse and human microsome preparations (t_{1/2}>1 hour) will be prioritized.

### Cytochrome P450 Inhibition.

Solubility. The maximum aqueous solubility of analogs will be initially determined by nephelometry using a published method (44). Particularly promising compounds will be confirmed using LC/MS as described.(45)

Protein binding. Plasma protein binding will be determined for mouse, rat, dog and human using equilibrium dialysis.(55).

Assessment of off-target effects of priority compounds. To assess potential off-target effects, we will evaluate prioritized hits validated by in vivo testing in the Eurofins Panlabs HitProfilingScreen+CYP450 assay panel, which is performed by Eurofins as a fee for service. The assay panel of consists of assays for 30 receptors and transporters plus the five major CYP450 enzymes (1A2, 2C19, 2C9, 2D6, 3A4). These assays will be used to verify that potential preclinical candidates do not exhibit potential off-target toxicities.

Formulation. For in vivo studies, MBX-5452 was formulated in a solution of either 0.9% saline (physiological saline) or 0.45% sodium bicarbonate with 1.5% polysorbate 80 (Tween 80). MBX-6681 was formulated in a solution of either 0.45% sodium bicarbonate with 5% cremophor EL.

Single dose Tolerability Determination in mice. CD-1 female mice, weighing 18-22 g received a single dose of the test agents at a pre-determined concentration via up to 3 predetermined routes of administration (IP, SC or IV). If the first animal appeared healthy, two additional animals were dosed at the same concentration and observed up to 48 h. For dose concentrations with no adverse events, the concentration was increased, and up to three naïve mice were dosed. This study scheme continued with escalating doses until an animal demonstrated an adverse effect to the dose administration or a defined upper dose concentration limit was reached. If an animal demonstrated adverse effects to the dose administration, the compound concentration was halved (1/2-X) and up to three animals were dosed and observed. The MTD was defined as the highest dose concentration that demonstrated no adverse effects after 48 h post-dose. Animals were observed continually for one hour post drug administration and at least 5 additional times during the 48 hour period (2 additional times during the first 24 hours and 3 observation periods between 24 and 48 hours). All animals that received doses of the test agent were euthanized at study termination (48 hrs.). If any animal exhibited adverse effects at any time post dose administration, they were removed from the study and euthanized. For these series of studies, the acute tolerability is not expected to differ between male and female mice; in an effort to increase efficiency and reduce the number of animals used, we only utilized female mice for the single dose tolerability.

Repeat dose Tolerability Determination in mice. Based on the results of the single dose tolerability study, a well tolerated dose concentration and route of administration was selected. CD-1 female and male mice (n≤10) were administered 2 or 3 doses a day (BID or TID) for up to 3 days. Pre-dose body weights and daily body weights were recorded during and 3 days after the last dose administration. Animals were observed during each dose event and up to 2 additional times per day. Observations occurred for a total of up to 6 days. Any animal that exhibited adverse effects at any time during the study was removed from the study and immediately euthanized. All remaining animals were terminated at the study conclusion.

Single dose Pharmacokinetic (PK) Determination in mice. CD-1 female mice weighing 18-22 g received a single dose of test article administered by up to three routes (IV, SC or IP) based on the single and repeat dose tolerability study results. At selected time points, 3 mice were euthanized and blood, lung, spleen, and/or liver samples were subsequently collected post euthanasia for analysis. Typically, each test article was evaluated for 7 time points (e.g., 0.083, 0.25, 1, 4, 6, 8, and 12 hr). Using the plasma concentrations from the bio-analysis, Tₘₐₓ, Cₘₐₓ, half-life, volume of distribution, and AUC were calculated. In addition, Tₘₐₓ, Cₘₐₓ, and AUC values were often calculated from epithelial lining fluid (ELF) levels determined from BAL (bronchoalveolar lavage) samples, as well as from lung, spleen, liver levels.

For these series of studies, the pharmacokinetic profile was not expected to differ between male and female mice. Accordingly, in an effort to increase efficiency and reduce the number of animals used, we only utilized female mice for the single dose PK study.

Repeat dose PK Determination in mice. CD-1 male and female mice weighing 18-22 g were dosed 2 or 3 times a day (BID or TID) for up to 3 days. The test article was administered by one route (IV, SC or IP) as a dose concentration based on the single and repeat dose tolerability studies. At selected time points, 5 mice were euthanized and subsequently blood and/or lung, BAL (bronchoalveolar lavage), liver, spleen samples were collected post euthanasia for analysis. For each test article, samples were collected for up to 24 time points collected for analysis over up to 2 days of test article administration and up to 3 additional days of observation.

Using the plasma concentrations from the bio-analysis, Tₘₐₓ, Cₘₐₓ, half-life, volume of distribution, and AUC were calculated. In addition, Tₘₐₓ, Cₘₐₓ, and AUC values were often calculated from epithelial lining fluid (ELF) levels determined from BAL samples, as well as from lung, spleen, liver levels.

Single dose Pharmacokinetic (PK) Determination in rats. Male Sprague Dawley rats from Charles River Laboratories were acclimated for 5 days prior to start of study. Animals were housed 3 per cage with free access to food and water during acclimation. Animals were individually identified through tail marking prior to weighing and dosing. All procedures were performed according to NeoSome IACUC policies and guidelines as well as OLAW standards. Test agent MBX-5452A was was formulated at 25 mg/mL in 5% PS80 in 0.9% saline. The test article was administered in a volume of 10 mL/kg via intraperitoneal or subcutaneous injection. Body weights, dose volumes, and times of dose administration were recorded. At each time point following dose administration, blood was collected through saphenous vein into K₂EDTA collection tubes and inverted several times to ensure thorough mixing. Blood was kept on wet ice until centrifugation (5 minutes, 8,000 RPM at 4° C.). Plasma was decanted into labeled tubes and stored at −80° C. until they were processed for bioanalysis. Using the plasma concentrations from the bio-analysis, Tₘₐₓ, Cₘₐₓ, half-life, volume of distribution, and AUC were calculated.

Non-neutropenic murine pneumonia model with P. aeruginosa infection-bacterial load. Immune-competent (non-neutropenic) BALB/c mice, male and female, were utilized for these studies. On Day 0, at 0 hour, each mouse was challenged with 5×10⁶ to 1×10⁷ CFUs of bacteria (e.g., a T3SS producing Pseudomonas aeruginosa strain such as PA99, grown in LB broth) via intranasal administration, as follows. Mice were anesthetized with isoflurane anesthesia, and once asleep, the mice were held in a vertical plane, and 50 uL of the prepared bacterial inoculum was placed drop-wise on the nares. Once the full volume of the inoculum was delivered, the mouse was returned to its home cage and allowed to recover from anesthesia. The number of CFUs in the inoculum was determined by the virulence of the bacterial isolate through previously performed bacterial titration studies. The bacterial challenge of mice was performed in a dedicated BSL-2 suite designed for containment of infectious agents. Test compounds were administered via a pre-determined route (IV, SC, or IP) in 5-20 mg/mL formulations described above beginning at 1 hour post infection, with additional doses administered by the same route at intervals of 6 hr (QID), 8 hr (TID), or 12 hr (BID) based on pharmacokinetic data to achieve compound levels above the EC₅₀ in appropriate tissues during the time between doses.

Animals were observed frequently for the next 24 hours for signs of pain and distress. Animals that appeared moribund, had hunched posture, were lethargic, or cold to the touch were removed from the study and immediately euthanized. Twenty-four hours after initiation of therapy (25 hours post infection), mice were euthanized via CO₂ inhalation, and appropriate tissues (lungs, spleen, and/or liver) were aseptically removed, weighed, homogenized and serially diluted. The diluted samples were plated on bacterial growth agar for CFU enumeration after overnight incubation. The average log 10 CFU and standard deviations were determined for each group of animals. Preferably, 8-10 mice per group were used to allow for the proper statistical analysis between treated and non-treated groups and the inherent variability of this model, allowing for standard deviations to be below a 0.5 log 10 CFU variation for any study group. A Mann-Whitney statistical test was performed (using GraphPad Prism software, GraphPad Software, LLC, San Diego, CA) between treated and infection control groups with a P-value of less than 0.05 considered significantly different. The study design included one infection control groups (at 1 hour), one or more experimental compound groups at up to 5 dose concentrations, a positive control agent (e.g., ciprofloxacin or meropenem) at up to 3 dose concentrations (to demonstrate significant CFU reduction or at sub-efficacious levels to demonstrate synergy or additivity with the test compound) for a total of up to 8 groups, N=10 each (e.g., up to 80 mice per study).

### Example 2. Improved In Vitro Properties of Zwitterionic T3SS Inhibitors

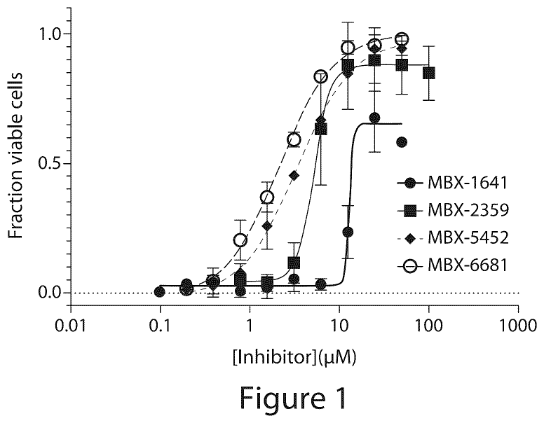

The results of these studies underscored the unexpected effect of zwitterionic moieties on the phenoxyacetamide T3SS inhibitor scaffold. As shown in Table 5, several zwitterionic PhA T3SS inhibitors (see Table 1 for structures) exhibit improved potency in the in vitro T3SS activity assays described above as compared to the earlier PhA compounds MBX-1641 and MBX-2359, especially in the T3SS translocation assay that measures the translocation of the pseudomonal exotoxins ExoS or ExoU into mammalian cells. For example, the lead compounds of this series, MBX-5452A and MBX-6681B (highlighted in Table 5), exhibit increased potencies in the in vitro T3SS translocation assay as compared to an earlier generation of inhibitors exemplified by MBX-2416 and MBX-2359 (see FIG. 1). As shown in Table 6, the EC₅₀s for MBX-5452A and MBX-6681B are 2-4 lower than those of MBX-1641 and MBX-2359. In addition, MBX-5452A and MBX-6681B exhibit potent T3SS inhibitory activities against a diverse panel of clinical isolates of P. aeruginosa that express exotoxins ExoS or ExoU and varying antibiotic resistance genes (see Tables 7 and 8). Several isolates in the strain panel are multidrug resistant (MDR). As shown in Table 7, MBX-5452A exhibited potent T3SS inhibitory against all the strains in panel of clinical isolates and the EC₅₀ of MBX-5452A for 90% of the 26 clinical strains tested was 1.9 μg/mL. Similarly, MBX-6681B exhibited potent T3SS inhibitory against all the strains in panel of clinical isolates and the EC₅₀ of MBX-6681B for 90% of the 22 clinical strains tested was 1.4 μg/mL. Clearly, the zwitterionic moieties on the PhA scaffold provide surprising improvements in inhibitory activity against the T3SS of P. aeruginosa.

While the zwitterionic moieties provide improved in vitro potency against T3SS activity assays, the most unexpected effect of these alterations to the PhA scaffold was the improvements in the predicted drug-like properties of these compounds underscored by the results of in vitro ADME experiments. Table 9 shows the results of a panel of in vitro ADME experiments for selected zwitterions shown in Table 2. Several zwitterions exhibited a surprisingly high level of aqueous solubility of ≥200 μM, undetectable mammalian cell cytotoxicity (CC₅₀≥200 μM), favorable levels of serum protein binding (<95% bound), stability in the presence of murine liver microsomes (T_{1/2}≥139 min) and liver hepatocyte cultures (T_{1/2}≥200 min), including the lead compounds MBX-5452A and MBX-6681B. Table 10 shows a comparison of the drug-like properties of two earlier PhA compounds (MBX-1641 and MBX-2359) and the lead compounds MBX-5452A and MBX-6681B. While limited data is available for MBX-1641 and MBX-2359, it is clear that the zwitterionic compounds exhibit a 4-8 fold increase in solubility and a >100-fold increase murine liver microsome stability. Finally, the zwitterionic substitutions resulted in unexpected improvements in the predicted safety of these compounds during use in humans, as demonstrated by the results from in vitro safety assays against a panel of human cytochrome P450s, receptors, and ion channels that was performed by PanLabs/Eurofins (Taipei, Taiwan). The results of these assays for MBX-5452A (assayed at 100 μM), MBX-6681 (assayed at 10 μM), and an earlier PhA compound MBX-4532 (assayed at 10 μM) are shown in Table 11. MBX-5452A did not exhibit >50% inhibition in any of the safety assays when tested at a concentration of 100 μM, whereas MBX-4532 exhibited >50% inhibition against 5 safety targets (highlighted in bold font) when tested at a concentration of 10 μM. MBX-6681 did not exhibit >50% inhibition in any of the safety assays when tested at a concentration of 10 μM. Clearly, the zwitterionic substitutions provide surprising improvements in in vitro assays for drug-likeness and safety as compared to the earlier PhA T3SS inhibitors.

### Example 3. Improved In Vivo Properties of Zwitterionic T3SS Inhibitors

The surprising improvements in drug-likeness and safety provided by the zwitterionic PhA substitutions translate into improvements in pharmacokinetics, tolerability, and efficacy in in a immunocompetent murine model of acute pneumonia.

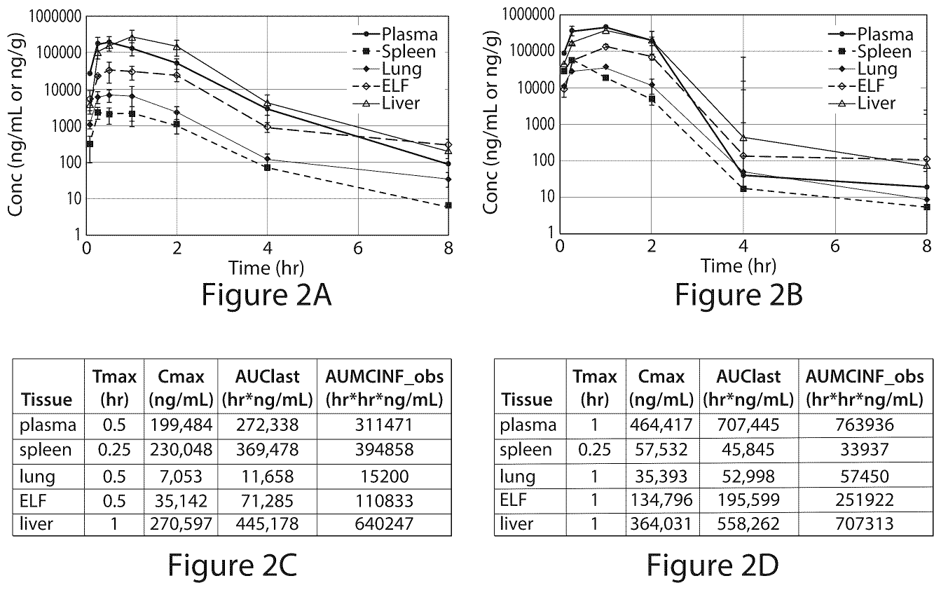

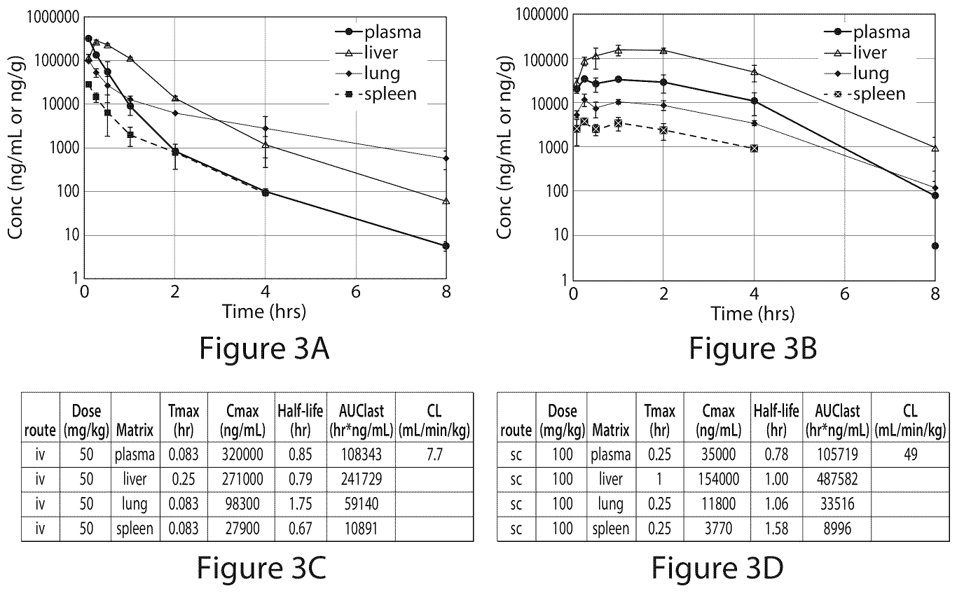

As shown in FIGS. 2A and B, the pharmacokinetics (PK) of MBX-5452A in mice after administration of single dose of 250 mg/kg via the subcutaneous (SC) or intraperitoneal (IP) routes of administration indicates excellent exposure in plasma (AUClast 272,338 hr*ng/ml) and relevant tissues for 5-6 and 3-4 hours after SC and IP administration, respectively. The concentration of MBX-5452A in plasma exceeds the EC₅₀ in the translocation assay for 90% of clinical isolates tested (1900 ng/mL) for 5-6 hours. The levels of compound in spleen, ELF, lung, and liver indicate that the compound distributes rapidly into tissues. Similarly, FIG. 3 4 illustrates the surprisingly favorable pharmacokinetic (PK) of MBX-6681B in mice after administration of single dose of 50 mg/kg via the intravenous (IV) and 100 mg/kg via subcutaneous (SC) routes of administration. The data demonstrate that MBX-6681 distributes rapidly into tissues after a 100 mg/kg SC dose and provides good exposure overall (AUClast 105,719 hr*ng/mL), which is approximately equivalent to the AUClast for MBX-5452A normalized by dose levels. The concentration of MBX-6681B in plasma remain above the EC₅₀ in the translocation assay for 90% of clinical isolates tested (1400 ng/mL) for 2-3 hours after a SC dose of 100 mg/kg. As shown in Figure, a PK analysis of repeated SC doses of 100 mg/kg TID (q8h) of MBX-5452A (Panel A) and MBX-6681B (Panel B) for 40 hours does not result in significant accumulation of compounds in plasma, ELF, or lung after repeated dosing, with peak plasma levels averaging around 100 μg/mL shortly after each administration. Finally, MBX-5452A exhibited favorable pharmacokinetics in rats after a single 250 mg/kg doses administered IP or SC (Panel A), and the calculated PK parameters are shown in Panel B. The plasma concentration curves from mice and Pk parameters after a single 250 mg/kg doses administered IP or SC are included in Panel A and B, respectively, for comparison. The data indicate that the PK parameters for rats and mice are similar, except that the half-life and MRTINF_obs are significantly higher in rats, and the concentration of MBX-5452A in plasma after SC administration are higher than the T3SS translocation EC₅₀ for >16 hours. Taken together, the zwitterionic PhAs exhibit favorable PK properties that are consistent with efficacy in an animal model of infection.

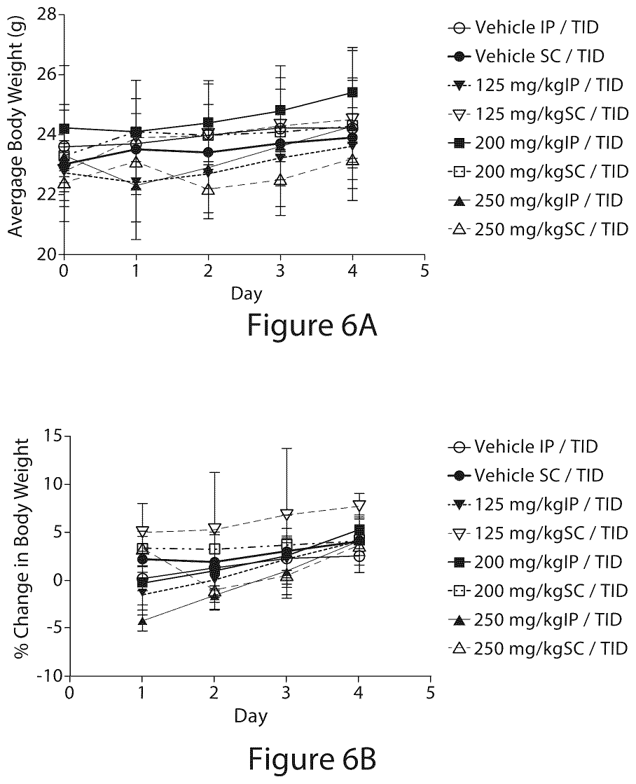

To determine whether multiple doses of MBX-5452A are tolerated by mice, a repeat-dose tolerability study was performed in which repeat SC or IP doses of 750 mg/kg/day (250 mg/kg, TID of MBX-5452A were administered to mice and body weight and clinical signs were monitored for 4 days. As shown in FIG. 6, repeat doses of MBX-5452A did not significantly affect body weight (Panel A) or percent change in body weight (Panel B) of CD-1 mice over the course of 4 days. In addition, no adverse events were reported during the course of the experiment, indicating that repeated dosing of MBX-5452A is well-tolerated by mice, consistent with the results of the in vitro safety experiments.

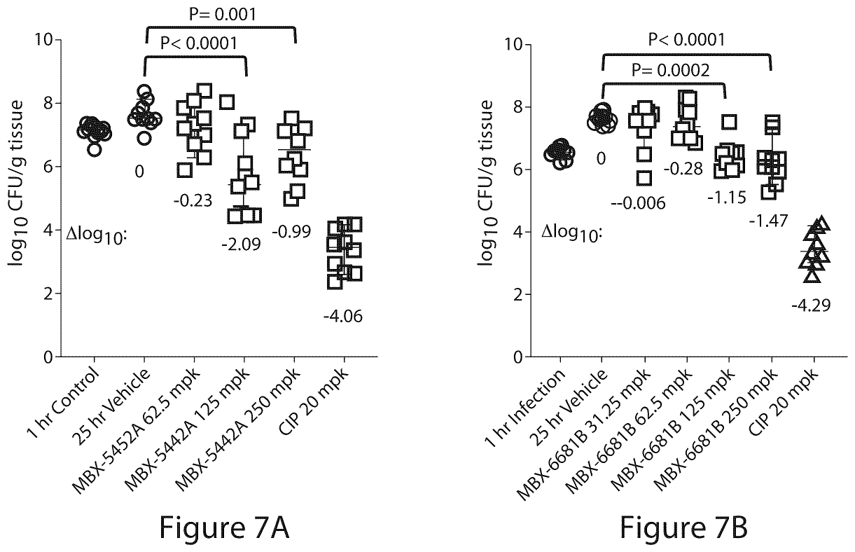

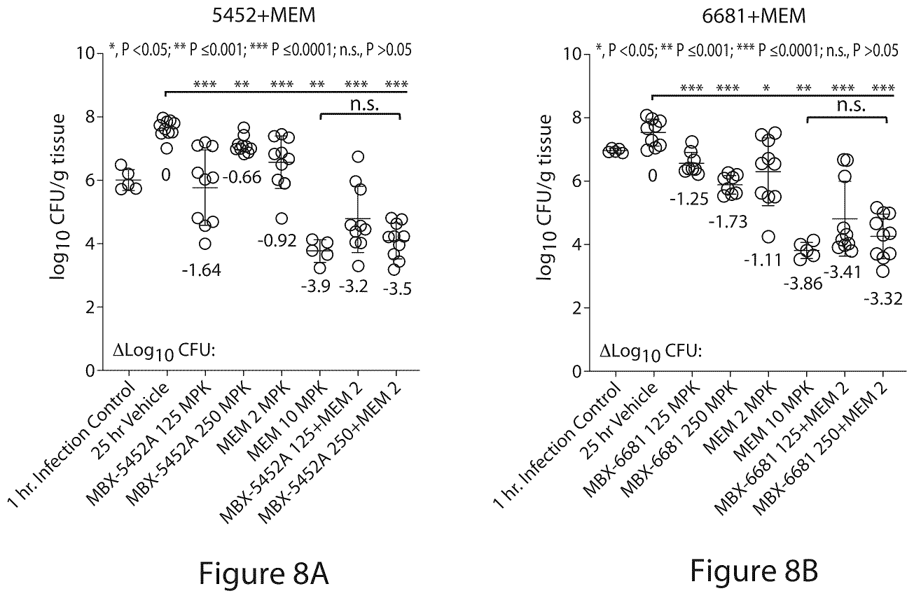

To determine whether the zwitterionic PhAs MBX-5452A and MBX-6681B are efficacious in a relevant animal model of infection, these compounds were used to treat an experimental infection in a immunocompetent murine model of acute pneumonia after challenge with P. aeruginosa PA99, either as a single agent or in combination with a subefficacious dose of the anti-pseudomonal antibiotic meropenem (MEM). In this model, CD-1 mice were infected via the intranasal route with 1×10⁷ bacteria, treatment was initiated 1 hour post-infection, and mice were sacrificed 24 hours post-infection and the bacterial load in the lungs was determined. As shown in FIG. 7, MBX-5452A (Panel A) and MBX-6681B (Panel B) significantly reduce bacterial load (CFUs/g lung tissue) by 1-2 Log₁₀ (P<0.0001) in the lung by as a single agent when administered at doses of 125 and 250 mg/kg TID. Importantly, the data in FIG. 8 demonstrates that MBX-5452A (Panel A) and MBX-6681 (Panel B) significantly increased the efficacy of a sub-efficacious dose of meropenem (MEM, 2 mg/kg TID) in decreasing the bacteria load in infected lungs to a level similar to that of the positive control (MEM, 10 mg/kg TID). Both compounds exhibited synergistic effects in combination with 2 mg/kg MEM, as the Log 10 reduction in CFU of the combinations were significantly greater than the sum of the Log 10 reductions of the individual treatments. These results provide proof-of-concept for the use of these compounds as adjunctive agents to anti-pseudomonal antibiotics to treat hospital-acquired and ventilator-associated pneumonia caused by P. aeruginosa.

TABLE 6
Surprising improvement in inhibitory activities of zwitterionic PhAs MBX-5452A and
MBX-6681B against in vitro T3SS activity assays as compared to earlier PhA analogs MBX-
1641 and MBX-2359.

| | | ExoS- | LUX | |
|---|---|---|---|---|
| | | BLA IC₅₀ | IC₅₀ | Translocation |
| MBX# | Structure | (μM) | (μM) | EC₅₀ (μM) |
| 1641 | 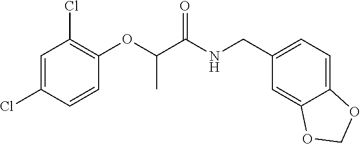 [SMILES: CC(Oc1ccc(Cl)cc1Cl)C(=O)NCc1ccc2c(c1)OCO2] | 10 | 4.7 | 15 |
| 2359 | 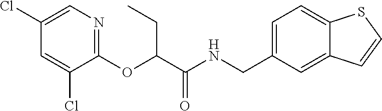 [SMILES: CCC(Oc1ncc(Cl)cc1Cl)C(=O)NCc1ccc2sccc2c1] | 1.5 | 1.3 | 5.2 |
| 5452A | 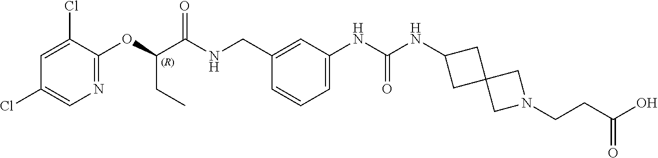 | 2.53 | 1.9 | 2.7 |
| 6681B | 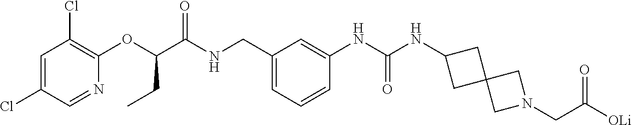 | 2.76 | 1.8 | 1.9 |

TABLE 7
Inhibitory activity of MBX-5452A in the in vitro T3SS translocation assay against a
panel of clinical isolates of Pseudomonas aeruginosa. The EC₅₀ of MBX-5452A for 90% of the
26 clinical strains tested is 1.9 μg/mL.

| Strain: | EC₅₀ | | | | Drug resistances | |
|---|---|---|---|---|---|---|
| MDM# | (μg/ml) | Source | Source# | Toxin | (phenotype) | Drug resistance genes |
| 1566 | 1.1 | Hauser | PA99 | ExoS + U | none | none |
| 2678 | 0.11 | CDC | 233 | ExoS | Azt, Dori, Imi, Mero | OXA-50, PAO, catB7 |
| 2681 | 1.52 | CDC | 236 | ExoU | Cipro, Levo, Imi, Gm, | OXA-50, aadB, PAO, aph(3′)- |
| | | | | | Toby | IIb |
| 2682 | 0.17 | CDC | 237 | ExoS | Imi, | OXA-50, PAO, catB7 |
| 2684 | 1.5 | CDC | 239 | ExoU | Ami, Azt, Cefep, Cftaz, | strB, aph(3′)-Ic, OXA- |
| | | | | | Cipro, Levo, Dori, | 50, tet(G), aadB, VIM-11, OXA- |
| | | | | | Mero, Imi, Gm, Toby, | 10, aac(6′)- |
| | | | | | Pip/tazo, Colisitin, & | IIa, strA, dfrB5, GES-1, cmlA1 |
| | | | | | Polymyxin B int, | |
| 2687 | 0.26 | CDC | 245 | ExoU | Ami, Azt, Cefep, Cftaz, | OXA-50, PAO |
| | | | | | Cipro, Dori, Mero, Imi, | |
| | | | | | Levo, Toby | |
| 2688 | 1.9 | CDC | 246 | ExoU | Ami, Azt, Cefep, Cftaz, | OXA-50, aadB, PAO, catB7 |
| | | | | | Cipro, Levo, Dori, | |
| | | | | | Mero, Imi, Gm, Toby, | |
| | | | | | Pip/tazo | |
| 2689 | 0.21 | CDC | 247 | ExoS | Imi | OXA-50, PAO, catB7 |
| 2692 | 1.2 | CDC | 250 | ExoU | Ami, Azt, Cefep, Cftaz, | OXA-50, PAO, catB7 |
| | | | | | Cipro, Levo, Dori, | |
| | | | | | Mero, Imi, Gm, Toby, | |
| | | | | | Pip/tazo | |
| 2694 | 0.44 | CDC | 253 | ExoS | Azt & Ceftaz | OXA-50, PAO, catB7 |
| | | | | | intermediate | |
| 2699 | 1.3 | CDC | 258 | ExoU | Cipro, Levo | OXA-50 |
| 2702 | 1.6 | CDC | 261 | ExoU | Cipro, Levo | OXA-50, PAO |
| 2710 | 0.74 | CDC | 269 | exoS | Cipro, Imi, Levo | OXA-50, PAO, catB7 |
| 2711 | 0.06 | CDC | 270 | ExoS | Cipro, Imi, Levo | OXA-50, PAO, catB7 |
| 2713 | 0.12 | CDC | 272 | ExoS | Cipro, Imi, Levo | OXA-50, PAO, catB7 |
| 2740 | 0.16 | Hauser | PABL-010 | ExoS | Zosyn, Levo, Ami, Gm | NA |
| 2744 | 0.14 | Hauser | PABL-018 | ExoS | Zosyn (piperacillin + | NA |
| | | | | | tazobactam) | |
| 2746 | 0.74 | Hauser | PABL-020 | ExoU | Zosyn, Levo, Ami, Gm | NA |
| 2753 | 1.64 | Hauser | D2 | exoU | NA | NA |
| 2756 | 1.84 | Hauser | S2 | exoU | NA | NA |
| 2757 | 1.94 | Hauser | S3 | exoU | NA | NA |
| 2758 | 1.02 | Hauser | S4 | exoU | NA | NA |
| 2759 | 1.24 | Hauser | S5 | exoU | NA | NA |
| 2760 | 1.07 | Hauser | S7 | exoU | NA | NA |
| 2766 | 2.61 | Hauser | S13 | exoU | NA | NA |
| 2767 | 0.49 | Hauser | S14 | exoS | NA | NA |
| 2769 | 0.53 | Hauser | S16 | exoS | NA | NA |

EC₅₀ vs. 90%: 1.9 μg/ml
Range: 0.06-2.61 μg/mL (n = 26)

TABLE 8
Inhibitory activity of MBX-6681B in the in vitro T3SS translocation assay against a
panel of clinical isolates of Pseudomonas aeruginosa. The EC₅₀ of MBX-6681B for 90% of the
22 clinical strains tested is 1.4 μg/mL.

| Strain: | EC₅₀ | | | | | |
|---|---|---|---|---|---|---|
| MDM # | (μg/ml) | Toxin | Source | Source# | Resistance phenotype | Resistance genotype |
| 1566 | 1.1 | ExoS + U | Hauser | PA99 | None | None |
| 2678 | 0.16 | ExoS | CDC | 233 | Azt, Dori, Imi, Mero | OXA-50, PAO, catB7 |
| 2681 | 1.4 | ExoU | CDC | 236 | Cipro, Levo, Imi, Gm, | OXA- |
| | | | | | Toby | 50, aadB, PAO, aph(3′)- |
| | | | | | | IIb |
| 2682 | 0.18 | ExoS | CDC | 237 | Imi | OXA-50, PAO, catB7 |
| 2687 | 1.1 | ExoU | CDC | 245 | Ami, Azt, Cefep, Cftaz, | OXA-50, PAO |
| | | | | | Cipro, Dori, Mero, Imi, | |
| | | | | | Levo, Toby | |
| 2688 | 0.37 | ExoU | CDC | 246 | Ami, Azt, Cefep, Cftaz, | OXA- |
| | | | | | Cipro, Levo, Dori, Mero, | 50, aadB, PAO, catB7 |
| | | | | | Imi, Gm, Toby, Pip/tazo | |
| 2689 | 0.25 | ExoS | CDC | 247 | Imi | OXA-50, PAO, catB7 |
| 2692 | 0.66 | ExoU | CDC | 250 | Ami, Azt, Cefep, Cftaz, | OXA-50, PAO, catB7 |
| | | | | | Cipro, Levo, Dori, Mero, | |
| | | | | | Imi, Gm, Toby, Pip/tazo | |
| 2694 | 0.55 | ExoS | CDC | 253 | Azt & Ceftaz intermediate | OXA-50, PAO, catB7 |
| 2699 | 1.25 | ExoU | CDC | 258 | Sensitive | OXA-50 |
| 2702 | 0.66 | ExoU | CDC | 261 | Cipro, Levo | OXA-50, PAO |
| 2704 | 2.1 | ExoU | CDC | 263 | Cipro & Gm intermediate | OXA-50 |
| 2711 | 0.11 | ExoS | CDC | 270 | Cipro, Imi, Levo | OXA-50, PAO, catB7 |
| 2713 | 0.14 | ExoS | CDC | 272 | Cipro, Imi, Levo | OXA-50, PAO, catB7 |
| 2740 | 0.44 | ExoS | Hauser | PABL-010 | Zosyn, Levo, Ami, Gm | n.d. |
| 2744 | 0.66 | ExoS | Hauser | PABL-018 | Zosyn (piperacillin + | n.d. |
| | | | | | tazobactam) | |
| 2753 | 1 | ExoU | Hauser | AH-D2 | n.d. (eye isolate) | n.d. |
| 2756 | 1.4 | ExoU | Hauser | AH-S2 | n.d. (blood isolate, | n.d. |
| | | | | | Taiwan) | |
| 2759 | 0.6 | ExoU | Hauser | AH-S5 | n.d. (blood isolate, | n.d. |
| | | | | | Taiwan) | |
| 2766 | 1.5 | ExoU | Hauser | AH-S13 | n.d. (blood isolate, | n.d. |
| | | | | | Taiwan) | |
| 2767 | 0.23 | ExoS | Hauser | AH-S14 | n.d. (blood isolate, | n.d. |
| | | | | | Taiwan) | |
| 2769 | 0.25 | ExoU | Hauser | AH-S16 | n.d. (blood isolate, | n.d |
| | | | | | Taiwan) | |

EC₅₀ vs. 90% = 1.4 μg/ml
Range = 0.11-2.1 μg/ml

TABLE 9
Activities of select zwitterionic PhA analogs in in vitro ADME assays.

| | Sol^{a} | Cytotox^{b} | Serum Bind^{c}- | Serum Bind^{d}- | HLMS^{e} | MLMS^{f} | MLHep^{g} |
|---|---|---|---|---|---|---|---|
| MBX# | (μM) | CC₅₀ (μM) | M (% bound) | H (% bound) | T_{1/2} (min) | T_{1/2} (min) | T_{1/2} (min) |
| 5414A | ≥200 | ≥200 | N.D. | N.D. | >139 | >139 | N.D. |
| 5427A | 100 | ≥200 | N.D. | N.D. | N.D. | N.D. | N.D. |
| 5430A | 200 | ≥200 | N.D. | N.D. | N.D. | N.D. | N.D. |
| 5442A | 200 | ≥200 | N.D. | N.D. | N.D. | N.D. | N.D. |
| 5452A | ≥200 | ≥200 | 89.3 | 97.4 | >139 | >139 | >231 |
| 5453A | 200 | ≥200 | 68.9 | 81.3 | >139 | >139 | N.D. |
| 5454A | 200 | ≥100 | N.D. | N.D. | N.D. | N.D. | N.D. |
| 5456A | 200 | 107 | N.D. | N.D. | N.D. | N.D. | N.D. |
| 5522A | ≥400 | N.D. | N.D. | N.D. | N.D. | N.D. | N.D. |
| 5523A | ≥400 | N.D. | 79.3 | 87.2 | >139 | >139 | N.D. |
| 5524A | ≥400 | N.D. | N.D. | N.D. | N.D. | N.D. | N.D. |
| 5525A | 150 | N.D. | 77.3 | 94.1 | >139 | >139 | >231 |
| 5526A | ≥400 | N.D. | N.D. | N.D. | N.D. | N.D. | N.D. |
| 5527A | ≥400 | N.D. | N.D. | N.D. | N.D. | N.D. | N.D. |
| 5529A | ≥400 | N.D. | N.D. | N.D. | N.D. | N.D. | N.D. |
| 5530A | ≥400 | ≥200 | 76.2 | 84.3 | >139 | >139 | 61.9 |
| 5532A | ≥400 | ≥200 | 74.6 | 83.9 | >139 | >139 | N.D. |
| 5533A | 300 | ≥200 | 84.2 | 91.6 | >139 | >139 | N.D. |
| 5548A | 200 | N.D. | N.D. | N.D. | N.D. | N.D. | N.D. |
| 6681B | 100 | ≥200 | 88 | N.D. | >125 | >125 | >240 |
| 7061A | 1.1 | N.D. | N.D. | N.D. | N.D. | N.D. | N.D. |
| 7062A | 3.5 | N.D. | N.D. | N.D. | N.D. | N.D. | N.D. |
| 7438A | N.D. | ≥200 | N.D. | N.D. | N.D. | N.D. | N.D. |
| 7476A | N.D. | ≥200 | N.D. | N.D. | N.D. | N.D. | N.D. |
| 7477A | N.D. | ≥200 | N.D. | N.D. | N.D. | N.D. | N.D. |
| 7478A | N.D. | ≥200 | N.D. | N.D. | N.D. | N.D. | N.D. |
| 7497A | N.D. | ≥200 | N.D. | N.D. | N.D. | N.D. | N.D. |
| 7531A | N.D. | ≥200 | N.D. | N.D. | N.D. | N.D. | N.D. |

Footnotes:
^{a}kinetic solubility in water (nephelometric assay);
^{b}cytotoxicity vs. HeLa cells;
^{c}Serum binding assay using murine (M) serum;
^{d}Serum binding assay using human (H) serum;
^{e}Stability in human liver microsomes (HLM);
^{f}Stability in murine liver microsomes (MLM);
^{g}Stability in the presence of murine liver hepatocyte cells.

TABLE 10
Surprising improvement in in vitro ADME properties of zwitterionic PhA analogs
MBX-5452A and MBX-6681B as compared to earlier PhA analogs MBX-1641 and MBX-2359

| | | Cy- | Serum | Serum | | LHS |
|---|---|---|---|---|---|---|
| | | to- | Bind- | Bind- | ML | T_{1/2} |
| | Solu- | tox | Mouse | ing-H | MS | (min)- |
| | bility | CC₅₀ | (% | (% | T_{1/2} | M. R, |
| Structure | (μM) | (μM) | bound) | bound) | (min) | GP, H |
| 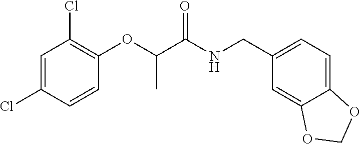 [SMILES: CC(Oc1ccc(Cl)cc1Cl)C(=O)NCc1ccc2c(c1)OCO2] | 50 | >100 | N.D. | N.D. | <1.0 | N.D. |
| 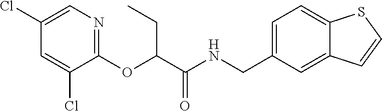 [SMILES: CCC(Oc1ncc(Cl)cc1Cl)C(=O)NCc1ccc2sccc2c1] | 25 | >100 | N.D. | N.D. | <1.0 | N.D. |
| 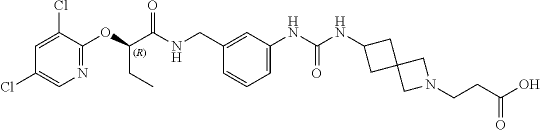 | >200 | >200 | 89 | 76 | >125 | >240 |
| 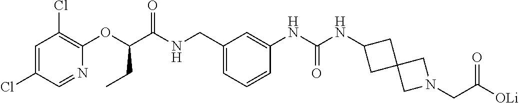 | >200 | >200 | 88 | 91 | >125 | >240 |

TABLE 11

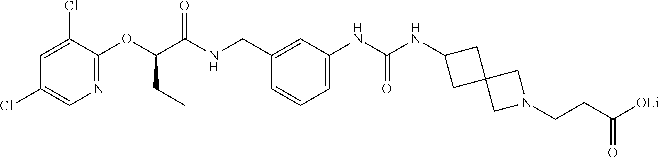

MBX-5452

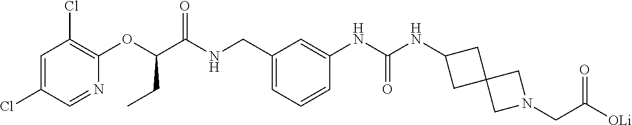

MBX-6681

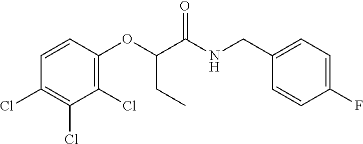 [SMILES: CCC(Oc1ccc(Cl)c(Cl)c1Cl)C(=O)NCc1ccc(F)cc1]

MBX-4532

| | MBX-5452A | MBX-6681 | MBX-4532 |
|---|---|---|---|
| Assay Name | (100 μM) | (10 μM) | (10 μM) |
| CYP450, 1A2 | 3 | 5 | 24 |
| CYP450, 2C19 | 24 | 8 | 80 |
| CYP450, 2C9 | 14 | 10 | −30 |
| CYP450, 2D6 | −16 | 0 | 0 |
| CYP450, 3A4 | −43 | −1 | 13 |
| Adenosine A1 | N.D. | 14 | 15 |
| Adenosine A2A | 1 | 8 | 10 |
| Adrenergic α1A | 9 | −9 | 12 |
| Adrenergic α1B | 5 | 6 | 6 |
| Adrenergic α2A | N.D. | −5 | 23 |
| Adrenergic β1 | −7 | 2 | 2 |
| Adrenergic β2 | −4 | 2 | 14 |
| Calcium Channel L-Type, Dihydropyridine | 14 | 12 | 56 |
| Cannabinoid CB1 | 8 | −3 | 16 |
| Dopamine D1 | −2 | 8 | 0 |
| Dopamine D2S | 4 | −2 | 11 |
| GABAA, Flunitrazepam, Central | 41 | 4 | −1 |
| GABAA, Muscimol, Central | N.D. | −3 | 2 |
| Glutamate, NMDA, Phencyclidine | 2 | −3 | 29 |
| Histamine H1 | 2 | −19 | 23 |
| Imidazoline I2, Central | N.D. | 24 | 27 |
| Muscarinic M2 | 6 | −2 | 23 |
| Muscarinic M3 | −10 | −7 | 8 |
| Nicotinic Acetylcholine α1, Bungarotoxin | N.D. | −9 | −17 |
| Nicotinic Acetylcholine αβ4 | 4 | 4 | 7 |
| Opiate μ (OP3, MOP) | 4 | 1 | 26 |
| Phorbol Ester | N.D. | −16 | 5 |
| Potassium Channel [KATP] | −3 | 9 | 0 |
| Potassium Channel hERG | 12 | 0 | 26 |
| Prostanoid EP4 | N.D. | 5 | −6 |
| Rolipram | N.D. | −10 | 23 |
| Serotonin (5-Hydroxytryptamine) 5-HT2B | 16 | −22 | 59 |
| Sigma σ1 | N.D. | −7 | 70 |
| Sodium Channel, Site 2 | −38 | 0 | 39 |
| Transporter, Norepinephrine (NET) | 3 | 6 | 61 |
| Cholinesterase, Acetyl, ACES | 9 | N.D. | N.D. |
| Cyclooxygenase, COX-1 | 5 | N.D. | N.D. |
| Cyclooxygenase, COX-2 | −4 | N.D. | N.D. |
| Monoamine Oxidase, MAO-A | 1 | N.D. | N.D. |
| Phosphodiesterase PDE3A | 3 | N.D. | N.D. |
| Phosphodiesterase PDE4D2 | 0 | N.D. | N.D. |
| Protein Tyrosine Kinase, LCK | 16 | N.D. | N.D. |
| Androgen (testosterone) | −7 | N.D. | N.D. |
| Cannabinoid CB2 | −44 | N.D. | N.D. |
| Cholecystokinin CCK1 (CCKA) | 7 | N.D. | N.D. |
| Endothelin ETA | 2 | N.D. | N.D. |
| Glucocorticoid | 6 | N.D. | N.D. |
| Histamine H2 | −6 | N.D. | N.D. |
| Muscarinic M1 | −2 | N.D. | N.D. |
| Opiate delta 1 (OP1, DOP) | 4 | N.D. | N.D. |
| Opiate kappa (OP1, KOP) | 2 | N.D. | N.D. |
| Serotonin 5-HT1A | 2 | N.D. | N.D. |
| Serotonin 5-HT1B | 2 | N.D. | N.D. |
| Serotonin 5-HT2A | 38 | N.D. | N.D. |
| Serotonin 5-HT2B | 16 | N.D. | N.D. |
| Serotonin 5-HT3 | 4 | N.D. | N.D. |
| Sodium Channel, Site 2 | −38 | N.D. | N.D. |
| Transporter, Dopamine (DAT) | 3 | N.D. | N.D. |
| Transporter, Norepinephrine (NET) | 3 | N.D. | N.D. |
| Transporter, Seritonen (SERT) | −4 | N.D. | N.D. |
| Vasopressin V1A | −26 | N.D. | N.D. |

What is claimed is:

1. A bacterial type III secretion system (T3SS) inhibitor compound of formula I: 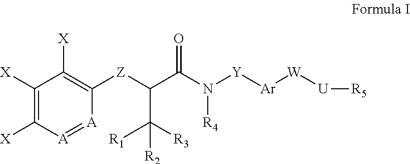
wherein,
A is independently selected from CH or N;
X is independently selected from hydrogen, halogen or hydroxyl;
Z is O, S, NH; or NR′, wherein R′ is alkyl;
R₁, R₂, and R₃ are independently selected from: hydrogen, halogen, alkyl, hydroxy, alkoxy, alkylthio, or cyano, wherein no more than two of the preceding radicals is hydrogen;
NR₄ wherein;
R₄ is hydrogen, a straight chain aliphatic group, a branched chain aliphatic group, cycloalkyl, haloalkyl, hydroxyalkyl, alkoxy, alkylamino, alkylthio, sulfonyl, sulfinyl, carboxy, alkoxycarbonyl or an aryl;
Y is selected from:
a divalent straight-chain, branched, or cyclic alkyl, alkenyl or alkynyl radical of from 1 to 6 carbon atoms, which may contain one or more heteroatoms, and which may be unsubstituted or substituted with up to four substituents selected from halo, cyano, hydroxy, amino, alkyl, cycloalkyl, alkylamino, carboxyl, alkoxycarbonyl, carboxamido, acylamino, amidino, sulfonamide, aminosulfonyl, alkylsulfonyl, aryl, heteroaryl, alkoxy, alkylthio; aryloxy, and heteroaryloxy; oxygen; oxime; or NR″ where R″ is hydrogen, alkyl or cycloalkyl;
Ar is an aryl or heteroaryl radical forming a five-membered or six-membered ring which may be additionally fused with from 1 to 3 aryl, heteroaryl, cycloalkyl, or heterocycloalkyl rings, which Ar radical may be unsubstituted or substituted with up to four substituents selected from halo, cyano, hydroxy, amino, alkyl, cycloalkyl, alkylamino, carboxyl, alkoxycarbonyl, carboxamido, acylamino, amidino, sulfonamido, aminosulfonyl, alkylsulfonyl, aryl, heteroaryl, alkoxy, alkylthio; aryloxy, and heteroaryloxy, and wherein any two substituents together may form an aromatic or non-aromatic ring structure fused with said aryl or heteroaryl radical Ar, or alternatively wherein substituents on Ar also may be optionally bonded covalently to either Y or R₄, or both Y and R₄, to form heterocyclic or carbocyclic ring systems, which ring systems may be aromatic, heteroaromatic, or partly aromatic, (that is, one or more rings being aromatic and one or more rings being non-aromatic (saturated));
W is a divalent radical bridging Ar and U selected from the group comprising, —COCH₂—, —SO₂—, —NHSO₂—, —SO2NH—, —CO—, —CH₂—, —CH(CH₃)—, —NHCO—, —NHCONH— —NCH₃CO—, —CONH—, —CONCH₃—, —O(CO)—, —(CO)O—, —NH—, or —O—;
U can be either a nitrogen or a carbon that is part of a non-aromatic heterocyclic ring of from 4-7 members and containing 1-2 nitrogen atoms, 0-1 oxygen atoms and bearing 0-3 substituents (in addition to the bond between W and the ring carbon/nitrogen and the U and the ring carbon in the formula) selected from alkyl, cycloalkyl, aryl, heteroaryl, haloalkyl, nitro, halogen, alkoxy, alkylthio, haloalkoxy, sulfonyl, or sulfinyl; and additionally U can be either a nitrogen or a saturated carbon that is linked via a chain of 1-4 carbon atoms to a basic nitrogen bearing, independently, hydrogen or aliphatic groups of less than nine carbon atoms that can be optionally substituted with groups selected from haloalkyl, nitro, halogen, alkoxy, alkylthio, haloalkoxy, sulfonyl, sulfinyl, carboxy, alkoxycarbonyl, or aminocarbonyl groups, heteroaryl, haloalkyl, nitro, halogen, alkoxy, alkylthio, haloalkoxy, sulfonyl, or sulfinyl;
R₅ is a monovalent radical selected from the group comprising, —CH2C(CH3)2COOH, —CH2COOH, —CH2COOH, —CH2CH2CH2COOH, —(CyBut)COOH, —CH2(CyPropyl)COOH, CH2(CyPropyl)COOH, —SO₂Me, —SO₂OH, —SO₂CF₃

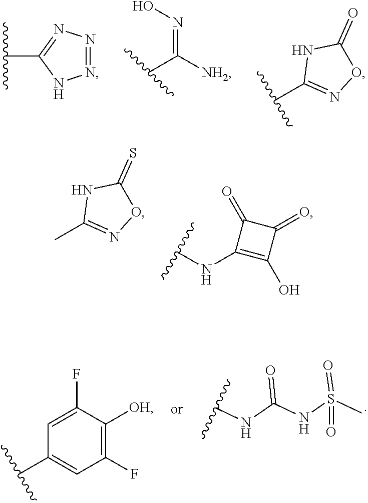

2. A bacterial type III secretion system (T3SS) inhibitor compound of formula I(a): 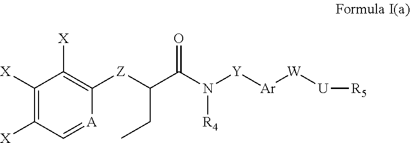
wherein:
A is independently selected from CH or N;
X is independently selected from hydrogen or halogen;
Z is O, S, NH; or NR′, wherein R′ is alkyl;
R₄ is hydrogen or methyl;
Y is selected from:
a divalent straight-chain, branched, or cyclic alkyl, alkenyl or alkynyl radical of from 1 to 6 carbon atoms, which may contain one or more heteroatoms, and which may be unsubstituted or substituted with up to four substituents selected from halo, cyano, hydroxy, amino, alkyl, cycloalkyl, alkylamino, carboxyl, alkoxycarbonyl, carboxamido, acylamino, amidino, sulfonamide, aminosulfonyl, alkylsulfonyl, aryl, heteroaryl, alkoxy, alkylthio; aryloxy, and heteroaryloxy; oxygen; oxime; or NR″ where R″ is hydrogen, alkyl or cycloalkyl;
Ar is an aryl or heteroaryl divalent radical forming a five-membered or six-membered ring which may be additionally fused with from 1 to 3 aryl, heteroaryl, cycloalkyl, or heterocycloalkyl rings, which Ar radical may be unsubstituted or substituted with up to four substituents selected from halo, cyano, hydroxy, amino, alkyl, cycloalkyl, alkylamino, carboxyl, alkoxycarbonyl, carboxamido, acylamino, amidino, sulfonamido, aminosulfonyl, alkylsulfonyl, aryl, heteroaryl, alkoxy, alkylthio; aryloxy, and heteroaryloxy, and wherein any two substituents together may form an aromatic or non-aromatic ring structure fused with said aryl or heteroaryl radical Ar, or alternatively wherein substituents on Ar also may be optionally bonded covalently to either Y or R₄, or both Y and R₄, to form heterocyclic or carbocyclic ring systems, which ring systems may be aromatic, heteroaromatic, or partly aromatic, (that is, one or more rings being aromatic and one or more rings being non-aromatic (saturated)).
W is a divalent radical bridging Ar and U selected from the group comprising, —COCH₂—, —SO₂—, —NHSO₂—, —SO2NH—, —CO—, —CH₂—, —CH(CH₃)—, —NHCO—, —NHCONH— —NCH₃CO—, —CONH—, —CONCH₃—, —O(CO)—, —(CO)O—, —NH—, or —O—;
U can be either a nitrogen or a carbon that is part of a non-aromatic heterocyclic ring system of between 4-7 members and containing 1-2 nitrogen atoms, 0-1 oxygen atoms and bearing 0-3 substituents (in addition to the bond between W and the ring carbon/nitrogen and the U and the ring carbon in the formula) selected from alkyl, cycloalkyl, aryl, heteroaryl, haloalkyl, nitro, halogen, alkoxy, alkylthio, haloalkoxy, sulfonyl, or sulfinyl; and additionally W can be either a nitrogen or a saturated carbon that is linked via a chain of 1-4 carbon atoms to a basic nitrogen bearing, independently, hydrogen or aliphatic groups of less than nine carbon atoms that can be optionally substituted with groups selected from haloalkyl, nitro, halogen, alkoxy, alkylthio, haloalkoxy, sulfonyl, sulfinyl, carboxy, alkoxycarbonyl, or aminocarbonyl groups, heteroaryl, haloalkyl, nitro, halogen, alkoxy, alkylthio, haloalkoxy, sulfonyl, or sulfinyl;
R₅ is a monovalent radical selected from the group comprising, —CH2C(CH3)2COOH, —CH2COOH, —CH2COOH, —CH2CH2CH2COOH, —(CyBut)COOH, —CH2(CyPropyl)COOH, CH2(CyPropyl)COOH, —SO₂Me, —SO₂OH, —SO₂CF₃

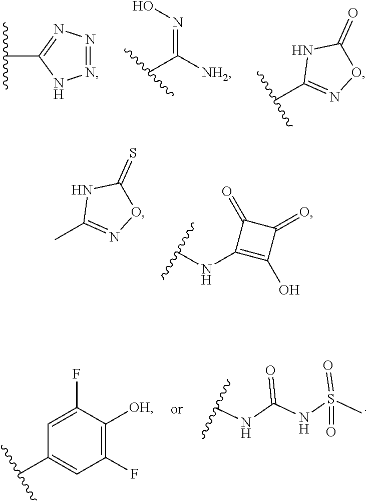

3. A bacterial type III secretion system (T3SS) inhibitor compound of formula I(b): 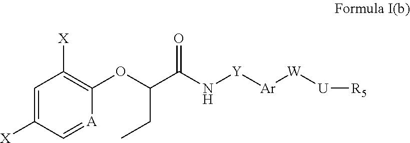
wherein,
A is CH or N;
X is independently selected from hydrogen or halogen;
Y is —CH₂—, —CH(CH₃)—, or —C(CH₃)₂—; and
Ar is an aryl or heteroaryl divalent radical forming a five-membered or six-membered ring which may be additionally fused with from 1 to 3 aryl, heteroaryl, cycloalkyl, or heterocycloalkyl rings, which Ar radical may be unsubstituted or substituted with up to four substituents selected from halo, cyano, hydroxy, amino, alkyl, cycloalkyl, alkylamino, carboxyl, alkoxycarbonyl, carboxamido, acylamino, amidino, sulfonamido, aminosulfonyl, alkylsulfonyl, aryl, heteroaryl, alkoxy, alkylthio; aryloxy, and heteroaryloxy, and wherein any two substituents together may form an aromatic or non-aromatic ring structure fused with said aryl or heteroaryl radical Ar, or alternatively wherein substituents on Ar also may be optionally bonded covalently to Y; to form heterocyclic or carbocyclic ring systems, which ring systems may be aromatic, heteroaromatic, or partly aromatic, (that is, one or more rings being aromatic and one or more rings being non-aromatic (saturated)).
W is a divalent radical bridging Ar and U selected from the group comprising, —COCH₂—, —SO₂—, —NHSO₂—, —SO2NH—, —CO—, —CH₂—, —CH(CH₃)—, —NHCO—, —NHCONH— —NCH₃CO—, —CONH—, —CONCH₃—, —O(CO)—, —(CO)O—, —NH—, or —O—;
U can be either a nitrogen or a carbon that is part of a non-aromatic heterocyclic ring system of between 4-7 members and containing 1-2 nitrogen atoms, 0-1 oxygen atoms and bearing 0-3 substituents (in addition to the bond between W and the ring carbon/nitrogen and the U and the ring carbon in the formula) selected from alkyl, cycloalkyl, aryl, heteroaryl, haloalkyl, nitro, halogen, alkoxy, alkylthio, haloalkoxy, sulfonyl, or sulfinyl; and additionally W can be either a nitrogen or a saturated carbon that is linked via a chain of 1-4 carbon atoms to a basic nitrogen bearing, independently, hydrogen or aliphatic groups of less than nine carbon atoms that can be optionally substituted with groups selected from haloalkyl, nitro, halogen, alkoxy, alkylthio, haloalkoxy, sulfonyl, sulfinyl, carboxy, alkoxycarbonyl, or aminocarbonyl groups, heteroaryl, haloalkyl, nitro, halogen, alkoxy, alkylthio, haloalkoxy, sulfonyl, or sulfinyl;
R₅ is a monovalent radical selected from the group comprising, —CH2C(CH3)2COOH, —CH2COOH, —CH2COOH, —CH2CH2CH2COOH, —(CyBut)COOH, —CH2(CyPropyl)COOH, CH2(CyPropyl)COOH, —SO₂Me, —SO₂OH, —SO₂CF₃

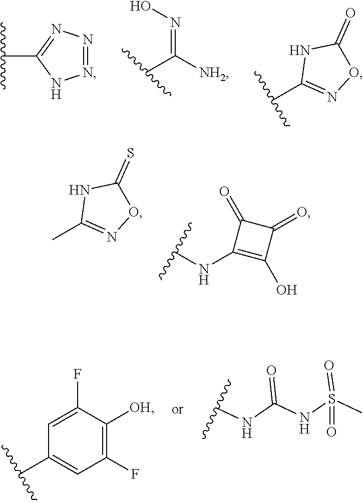

4. A bacterial type III secretion system (T3SS) inhibitor compound of Formula I(c): 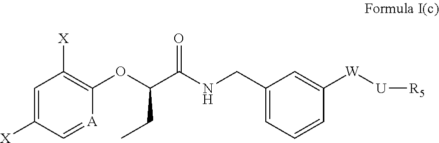
wherein:
A is CH or N;
at least one X is Cl and the other X is hydrogen, F, or Cl;
W is a divalent radical bridging Ar and U selected from the group comprising, —COCH₂—, —SO₂—, —NHSO₂—, —SO2NH—, —CO—, —CH₂—, —CH(CH₃)—, —NHCO—, —NHCONH— —NCH₃CO—, —CONH—, —CONCH₃—, —O(CO)—, —(CO)O—, —NH—, or —O—;
U can be either a nitrogen or a carbon that is part of a non-aromatic heterocyclic ring system of between 4-7 members and containing 1-2 nitrogen atoms, 0-1 oxygen atoms and bearing 0-3 substituents (in addition to the bond between W and the ring carbon/nitrogen and the U and the ring carbon in the formula) selected from alkyl, cycloalkyl, aryl, heteroaryl, haloalkyl, nitro, halogen, alkoxy, alkylthio, haloalkoxy, sulfonyl, or sulfinyl; and additionally W can be either a nitrogen or a saturated carbon that is linked via a chain of 1-4 carbon atoms to a basic nitrogen bearing, independently, hydrogen or aliphatic groups of less than nine carbon atoms that can be optionally substituted with groups selected from haloalkyl, nitro, halogen, alkoxy, alkylthio, haloalkoxy, sulfonyl, sulfinyl, carboxy, alkoxycarbonyl, or aminocarbonyl groups, heteroaryl, haloalkyl, nitro, halogen, alkoxy, alkylthio, haloalkoxy, sulfonyl, or sulfinyl;
R₅ is a monovalent radical selected from the group comprising, —CH2C(CH3)2COOH, —CH2COOH, —CH2COOH, —CH2CH2CH2COOH, —(CyBut)COOH, —CH2(CyPropyl)COOH, CH2(CyPropyl)COOH, —SO₂Me, —SO₂OH, —SO₂CF₃

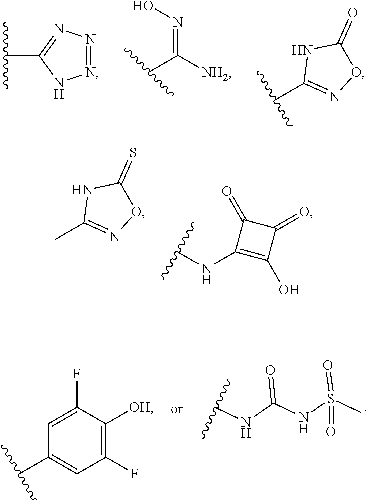

5. A method for treating or preventing a bacterial infection in a mammalian subject comprising administering a compound of Formula I, Formula I(a), Formula I(b), or Formula I(c).

6. The method according to claim 5 wherein the bacterial infection is caused by Pseudomonas.

7. The method according to claim 6 wherein the bacterial infection is caused by Pseudomonas aeruginosa.

8. The method according to claim 5 wherein the mammal is a human.
